(19) United States
(12) Patent Application Publication
(10) Pub. No.: US 2014/0242012 A1
(43) Pub. Date: Aug. 28, 2014

(54) METHODS FOR TREATING UV-DAMAGED SKIN AND SCC TUMORS AND FOR REMOVING TATTOOS WITH TOPICAL INGENOL MEBUTATE

(71) Applicant: LEO Laboratories Limited, Dublin (IE)

(72) Inventors: Sarah-Jane Cozzi, Herston (AU); Andreas Suhrbier, Bunya (AU); Steven Martin Ogbourne, Pinbarren (AU)

(21) Appl. No.: 14/139,869

(22) Filed: Dec. 23, 2013

Related U.S. Application Data
(63) Continuation of application No. PCT/IB2011/001910, filed on Jun. 27, 2011.
(60) Provisional application No. 61/501,253, filed on Jun. 26, 2011.
(60) Provisional application No. 61/501,171, filed on Jun. 24, 2011.

(51) Int. Cl. A61K 31/22; A61Q 19/02; A61K 9/00; A61K 8/37

(52) U.S. Cl. CPC A61K 31/22; A61K 8/375; A61Q 19/02; A61K 9/0014
USPC 424/62; 514/511

(57) ABSTRACT

The present invention is directed to the prophylatic field treatment of photodamaged skin with topical ingenol mebutate. More specifically, the present invention concerns field-directed treatment of UV-damaged skin with topical ingenol mebutate for reducing the number of skin lesions that emerge from the UV-damaged skin over time. In addition, the present invention concerns field-directed treatment for removing photodamaged skin, mutated keratinocytes, cutaneous immunosuppressive environments and/or p53+ patches caused by UV with topical ingenol mebutate. By way of example, the present invention is directed to treating photodamaged skin with topical ingenol mebutate at about 0.05% concentration.

The present invention is also concerned with the treatment of SCC tumors with topical ingenol mebutate for reducing the number of SCC tumors. By example, the present invention is directed to treating and curing SCC xenografts with topical ingenol mebutate at about 0.25% concentration.

The present invention is further directed to a topical field-directed treatment for the removal of tattoos from skin with ingenol mebutate. By way of example, the present invention is directed to removing tattoos with topical ingenol mebutate at concentrations of up to about 0.25%.

### RELATED APPLICATIONS

This application is a continuation of PCT Patent Application No. PCT/IB2011/001910, filed Jun. 27, 2011, which claims the benefit of and priority to U.S. Provisional Patent Application Ser. No. 61/501,253, filed Jun. 26, 2011, and U.S. Provisional Patent Application Ser. No. 61/501,171, filed Jun. 24, 2011. The contents of each of the foregoing applications are incorporated herein by reference in their entirety.

### FIELD OF THE INVENTION

The present invention is directed to the prophylatic field treatment of photodamaged skin with topical ingenol mebutate. More specifically, the present invention concerns field-directed treatment of UV-damaged skin with topical ingenol mebutate for reducing the number of skin lesions that emerge from the UV-damaged skin over time. In addition, the present invention concerns field-directed treatment for removing photodamaged skin, mutated keratinocytes, cutaneous immunosuppressive environments and/or p53+ patches caused by UV with topical ingenol mebutate. By way of example, the present invention is directed to treating photodamaged skin with topical ingenol mebutate at about 0.05% concentration.

The present invention is also concerned with the treatment of SCC tumors with topical ingenol mebutate for reducing the number of SCC tumors. By example, the present invention is directed to treating and curing SCC xenografts with topical ingenol mebutate at about 0.25% concentration.

The present invention is further directed to a topical field-directed treatment for the removal of tattoos from skin with ingenol mebutate. By way of example, the present invention is directed to removing tattoos with topical ingenol mebutate at concentrations of up to about 0.25%.

### BACKGROUND

According to the U.S. Department of Health and Human Services and the World Health Organization, ultraviolet (UV) radiation, from the sun and from tanning beds, is classified as a human carcinogen.

Scientists classify UV radiation generally into three types or bands, i.e., UVA, UVB and UVC. Even though the stratospheric ozone layer absorbs some of the harmful UV emitted from the sun, it does not screen all UV radiation. For example, while UVA, which is emitted at wavelength 320-400 nm, is not absorbed by the ozone layer, UVB, which is emitted at wavelength 290-320 nm, is mostly absorbed by the ozone layer, but some nevertheless does reach the Earth's surface. UVC, which is emitted at wavelength 100-290 nm, is generally believed to be completely absorbed by the ozone layer and atmosphere.

UVA and UVB radiation that reaches the Earth's surface contributes to the serious health effects listed above; it also contributes to environmental impacts. Levels of UVA radiation are more constant than UVB, reaching the Earth's surface without variations due to the time of day or year. UVA radiation is not filtered by glass.

The sun emits energy over a broad spectrum of wavelengths: visible light that you see, infrared radiation that you feel as heat, and UV radiation that you can't see or feel. UV radiation has a shorter wavelength and higher energy than visible light. It affects human health both positively and negatively. Short exposure to UVB radiation generates vitamin D, but can also lead to sunburn depending on an individual's skin type. As indicated above, while the stratospheric ozone layer shields life on Earth from most UV radiation, what does get through the ozone layer can cause numerous health problems, particularly for people who spend unprotected time outdoors or who are at greater risk to UV exposure. Such problems include skin cancer, cataracts, suppression of the immune system and premature aging of the skin.

Because the benefits of sunlight cannot be separated from its damaging effects, it is important to understand the risks of overexposure.

Sunlight causes photodamage to skin which in turn causes it to age faster than it should. Thus, skin age and a person's age may not necessarily be the same. Photodamaged or sun-damaged skin is something that few people escape in their lifetime. Photodamage results from exposure to sunlight or other sources of UV such as tanning beds, whether or not sun-tanning is involved. Approximately twenty five percent of lifetime UV exposure generally happens before people reach the age of twenty. UV-damaged or photodamaged skin manifests in numerous ways, such as advanced aging or wrinkling, thickening of the skin, i.e., the leathery, weather-beaten, elephant hide look (skin will generally thicken all over when people sun bake), uneven or pebbly skin, flabbiness, lifeless skin, pigmentation irregularities, small dilated blood vessels or red markings on or near the surface of the skin also known as telangiectasias, rough or scaly patches, e.g., actinic keratoses, freckles otherwise known as ephilides, liver spots, age spots, dark spots or skin tags known as lentigines, pre-skin cancers, and skin cancer, such as non-melanoma skin cancer (NMSC), e.g., superficial basal cell carcinoma (sBCC) and squamous cell carcinoma (SCC), and malignant melanoma.

Generally, these changes occur more frequently on areas that experience chronic exposure, such as the face, head, neck, chest, ears, arms, hands, backs and legs. Because the buttocks and upper inner arms are often unexposed, these areas of skin generally remain pristine evidencing the difference between chronologic aging and photoaging.

As the manifestations of photodamage intensify with age, it is paramount to seek medical advice and treatment, preferably early on, to mitigate and even possibly reverse some of the effects of photodamage to skin.

Non-melanoma skin cancers are the most common cancers worldwide, with the incidence increasing by 3-8% annually (Madan et al., 2010; Rogers et al., 2010). The direct cost of treating NMSC in the USA in 2004 was estimated to be $ 1.4 billion in U.S. dollars (The Lewin Group, 2005) with over 2 million patients treated in 2006 (Society, 2010). UV radiation, usually from sunlight, is the most important risk factor for skin cancer, but despite increased public awareness of the dangers of sun exposure, the incidence continues to rise (Madan et al., 2010; Rogers et al., 2010). The reasons for this increase likely include better detection of the disease, the aging population (Madan et al., 2010), and increasing UV radiation due to ozone depletion (Chang et al., 2010; Norval et al., 2007). Sunlight, and particularly UVB, is able to act both as a tumor initiator and a tumor promoter, with UVB able to damage DNA directly (Madan et al., 2010; Rass and Reichrath, 2008; Ziegler et al., 1994).

Mutations of the p53 tumor suppressor gene are particularly common in skin cancers, and are considered to be an important early event in skin cancer oncogenesis (Benjamin et al., 2008; de Gruijl and Rebel, 2008; Rebel et al., 2005). Chronic UV exposure results in the accumulation in the skin of histologically detectable “p53 patches”, which are clonal outgrowths of keratinocytes with elevated nuclear expression of mutated p53 (Berg et al., 1996; Rebel et al., 2005; Rebel et al., 2001). A small number of these mutant p53 patches may progress to actinic keratosis (Einspahr et al., 1999) and ultimately NMSC, with further mutations, UV-induced immunosuppression and/or human papilloma virus infection contributing to cancer formation (Byrne et al., 2008; Hart et al., 2001; Madan et al., 2010; Murphy, 2009; Rebel et al., 2005). Although a number of factors can influence the propensity of these mutated keratinocytes to develop into NMSC (Madan et al., 2010), these UV-mutated cells appear to be a pre-requisite for the development of most NMSC (Ananthaswamy et al., 1998; Benjamin et al., 2008; Rass and Reichrath, 2008). Consistent with this view is that treatments, such as sunscreen application, that reduce the number of p53 patches, also reduce the number of skin cancers that subsequently develop (Ananthaswamy et al., 2002; Conney et al., 2008).

The hairless, immunocompetent SKH1/hr mouse model has been reported in the literature as a model for UVB-induced photodamage, ultimately leading to the development of UVB-induced lesions including actinic keratosis (AK), squamous papillomas and squamous cell carcinoma (SCC) (Cozzi and Suhrbier, 2010). UVB wavelengths emitted by the sun are absorbed by the skin causing erythema, burns, immunosuppression, and DNA damage. The dual ability of UVB to cause malignant transformation of epidermal cells and immunosupression is thought to contribute to the development of cutaneous malignancies (Ch'ng et al., 2006). Chronic exposure of SKH1/hr mice to UVB leads to the development of “UV-signature” p53 mutations at hotspot codons (Benjamin et al., 2008; de Gruijl and Rebel, 2008). Clonal expansion of keratinocytes with p53 mutations leads to the formation of p53 mutant (p53+) patches/foci, which can be detected by immunohistochemistry (IHC) using an antibody that recognises the conformation of mutated p53 (Benjamin et al., 2008; de Gruijl and Rebel, 2008). Although some p53+ keratinocytes spontaneously regress in SKH1/hr mice (as they do in humans), others develop into pre-cancerous lesions resembling AKs and can progress to become SCCs (Rebel et al., 2001).

The outbred SKH1/hr model of UVB-induced p53+ mutations have been established at QIMR (Cozzi and Suhrbier, 2010). It has been shown that irradiating mice with 1.25 MED three times per week for 8 weeks leads to the formation of p53+ foci, which can be detected by IHC on epidermal sheets (Cozzi and Suhrbier, 2010). Quantitative analysis of p53+ patches over an area of 5 cm² of epidermis showed about 300 p53+ patches (Cozzi and Suhrbier, 2010). Two weeks after cessation of UVB irradiation, the size and number of p53+ patches had reduced, but >150 p53+ patches per 5 cm² remained (Cozzi and Suhrbier, 2010).

Patients with squamous cell carcinoma in situ (SCCIS) have a 3-5% risk of progression to invasive cancer. Treatment options include surgical excision, cryosurgery, curettage and cautery, and nonsurgical treatments, such as photodynamic therapy, lasers, 5-fluorouracil and imiquimod. See Moreno G, Chia A L, Lim A, Shumack S. Australas J Dermatol 2007; 48: 1-8.

The treatment option selected usually depends on lesion characteristics, such as size and site, or patient characteristics and preferences, such as age, medication, frailty, side effects, wound healing, cosmetic outcome and cost effectiveness. See Moreno G, Chia A L, Lim A, Shumack S. Australas J Dermatol 2007; 48: 1-8; Cox N H, Eedy D J, Morton C A; Therapy Guidelines and Audit Subcommittee, British Association of Dermatologists. Br J Dermatol 2007; 156: 11-21; and Kossard S, Rosen R H. J Am Acad Dermatol 1992; 27: 406-10.

Surgical options carry a significant risk of scarring, dehiscence, infection and hemorrhage.

SCCIS typically occurs on the lower legs of older women. See Kossard S, Rosen R H. J Am Acad Dermatol 1992; 27: 406-10. Underlying conditions such as lipodermatosclerosis and peripheral vascular disease in this group of patients make surgical options less attractive, with clinicians being wary of the risk of complications. Lack of skin mobility may limit excision of these lesions and poor wound healing is of concern. See Moreno G, Chia A L, Lim A, Shumack S. Australas J Dermatol 2007; 48: 1-8; Cox N H, Eedy D J, Morton C A; Therapy Guidelines and Audit Subcommittee, British Association of Dermatologists. Br J Dermatol 2007; 156: 11-21; and Ball S B, Dawber R P. Australas J Dermatol 1998; 39: 63-8.

Topical pharmacotherapy may therefore be advantageous in patients with lower limb lesions and in those wishing to avoid surgery. Current topical therapies require a long treatment duration and have poorly tolerated side effects.

Over the past couple of decades, tattoos have become part of mainstream culture. For example, it has been estimated that more than 10 million Americans have at least one tattoo, and that there are about 4,000 tattoo parlors now in business in the United States. In 2007, The Pew Research Centre reported that in the U.S., about 36% of 18-25 year olds and 40% of 26-40 year olds had at least one tattoo. In 2010 Harris Interactive estimated that 14% of Americans have at least one tattoo, with the incidence increasing to 32% in individuals under the age of 30.

Tattoos are made-up of small pigment particles deposited within the dermis layer of skin. The particles are frequently intracellular, but extracellular aggregates are also present. See Pfirrmann G, Karsai S, Roos S, Hammes S, Raulin C. Tattoo removal—state of the art. J Dtsch Dermatol Ges 2007 October; 5(10):889-97. According to the U.S. Food and Drug Administration, tattoo inks and pigments are listed as “color additives” for skin and are intended only for application to the top layer of the skin. Other than this designation by the FDA, tattoo inks and pigments remain largely unregulated.

In 2007 The Pew Research Centre reported that in the US 36% of 18-25 year olds and 40% of 26-40 year olds had at least one tattoo. See How Young People View Their Lives, Futures and Politics. A Portrait of “Generation Next”. The Pew Research Centre for the People and the Press 2007 Jan. 9 Available from: URL: http://people-press.org/report/300/a-portrait-of-generation-next. In 2010 Harris Interactive estimated that 14% of Americans have at least one tattoo, with the incidence increasing to 32% in individuals under the age of 30. See Regina a.Corso. Three in Ten Americans with a Tattoo Say Having One Makes Them Feel Sexier. Harris Interactive 2010 Feb. 12 Available from: URL: http://www.harrisinteractive.com/vault/Harris-Interactive-Poll-Research-Three-in-Ten-Americans-with-a-Tattoo-Sav-Havino-One-Makes-Them-Feel-Sexier-2008-02.pdf. Today, tattoos are generally applied with an electric tattoo machine equipped with needles that rapidly puncture the skin with an up and down motion between about 50 and 3,000 times per minute, somewhat like a sewing machine. With each puncture, the needle penetrates the top layer of the skin to a depth of about a millimeter to deposit a drop of insoluble ink or pigment to create the tattoo.

Notwithstanding the current popularity of tattoos in modern-day culture, it is estimated that as many as 50% of those with tattoos have some form of regret. See Suzanne L. Kilmer, Richard E. Fitzpatrick, Michael P. Goldman. Tattoo lasers. emedicine 2008 Jun. 25. Available from: URL: www.emedicine.com/derm/topic563.htm. As to why they have regrets, one in five (about 20%) say it's because they were too young when tattooed, while approximately 19 percent say it is because the tattoo is permanent and they are marked for life. About 18% regret their tattoos because they simply don't like them, while about 16% regret their tattoos because, over time, they fade. See The Harris Poll #15: Three in Ten Americans with a Tattoo Say Having One Makes Them Feel Sexier, Feb. 12, 2008. In a 2004 poll conducted by HI Europe and the U.S. parent-company Harris, it showed that 16% of Americans, 19% of Britons and 11% of Italians who were tattooed suffered regret. See Harris Interactive Europe poll: Americans and Britons Likelier Than Italians To Regret Decision to be Tattooed, Feb. 5, 2004.

Unfortunately, tattoos are meant to be permanent and, thus, removal can prove to be very difficult and painful. If, however, the tattoo ink or pigment is evenly injected at the same level by a skilled tattoo artist, i.e., about 1 mm within the top layer of skin, the tattoo may be somewhat easier to remove. Nonetheless, dermatologic surgeons usually warn that tattoo removal is painful and that complete tattoo removal may not be possible.

In the past, invasive techniques, such as surgical excision, dermabrasion (or sanding), salabrasion, cryosurgery and chemical peels (such as topical acids and silver nitrate), were used to remove tatoos. Those techniques are still presently used in certain circumstances to remove tattoos. However, pain and the high risk of scarring makes these methods unfavorable choices in most circumstances. See Burris K, Kim K. Tattoo removal. Clin Dermatol 2007 July; 25(4):388-92.

In the late 1980s, advances in procedures to remove tattoos were made. For example, medical lasers and infrared coagulation techniques were developed to remove tatoos, as alternatives to the invasive and scar-inducing procedures described above.

Medical lasers rely upon short pulses of intense beams of light that penetrate harmlessly through the top layers of the skin for absorption by the tattoo inks or pigments to significantly lighten or completely remove the tattoo, whether the tattoo was applied with colored or black inks. The laser light causes decomposition of the tattoo ink or pigment by thermal decomposition, or thermolysis, caused by heat. The absorbed laser light causes the tattoo ink or pigment to fragment into very small particles, which are then phagocytosed by macrophages and expelled via the lymphatic system. See Pfirrmann G, Karsai S, Roos S, Hammes S, Raulin C. Tattoo removal—state of the art. J Dtsch Dermatol Ges 2007 October; 5(10):889-97. This process generally takes a few weeks. Although scarring has been reduced using medical lasers, the removal of tattoos often requires numerous sessions, and sometimes the tattoo is not completely removed. See Pfirrmann G, Karsai S, Roos S, Hammes S, Raulin C. Tattoo removal—state of the art. J Dtsch Dermatol Ges 2007 October; 5(10):889-97.

Infrared coagulation (IRC), unlike medical lasers, uses infrared light to penetrate the skin layer to reach the tattoo. Unfortunately, infrared tattoo removal is known as the burn method for removing tattoos. Generally speaking, the tattoo ink or pigment is actually burned away for removal by the immune system. Thus, when the IRC method is selected to remove a tattoo, the treated area may blister, depending on the color of the ink. Notwithstanding this disadvantage associated with IRC tattoo removal, unlike laser removal, ink color is of no consequence. IRC will remove all tattoo ink or pigment colors, whereas laser removal may not.

Although the invasive techniques described above are still in use to remove tattoos, the medical laser and IRC procedures have become the gold standard for tattoo removal today; most likely due to the fact that they are non-invasive with fewer side effects.

The medical laser and IRC procedures are scheduled on an outpatient basis in a single or series of visits, which may or may not require topical or local anesthesia. Under either procedure, the actual treatments can take from about 10 minutes to about 30 minutes or longer, and the number of treatments for result is generally based upon the location, size, depth, and color of the tattoo, and the individual's ability to heal, the fragility of the individual, how the tattoo was applied, the type of ink or pigment used, and how long the tattoo has been in place. Following either procedure, a topical anti-bacterial pharmaceutical ointment and dressing are normally applied to the treated area to minimize infection. Treatments are generally spaced at least about 3 weeks apart to allow for the immune system to clear the fragmented tattoo ink or pigment. Results are typically not observed until at least about 5 weeks after treatment

Even though medical laser and IRC procedures are the gold standard today, they are not without drawback. Both techniques can be quite painful. The pain is often described as being splattered with hot oil, such as hot specks of bacon or other grease, on the skin or getting snapped by rubber bands. Following treatment and during the recovery period, the pain is associated with that of sunburn and the treated areas can swell, form scabs, form large bullas and turn reddish for weeks.

Side effects of the laser and infrared techniques include hyperpigmentation or hypopigmentation and textural changes to the skin. Hyperpigmentation is where dark spots can form, i.e., there is an abundance of color in the skin at the treatment site. Hypopigmentation, or a whitening of the skin, is where the treated area looses normal skin color. In addition, burns may result in scarring and a “paradoxical darkening” of the treated tattoo may occur, i.e., the treated tattoo becomes darker instead of lighter. This paradoxical darkening phenomenon typically occurs with flesh tone, pink, and cosmetic make-up tattoos. See, for example, S. Varma, et al.: Tattoo ink darkening of a yellow tattoo after Q-switched laser treatment. Clinical and Experimental Dermatology. 2002: Volume 27 Issue 6, pp. 461-463; and Holzer A, et al. Adverse Effects of Q-Switched Laser Treatment of Tattoos. Dermatologic Surgery 2007: Volume 34 Issue 1, pp. 118-122. Other possible side effects include infection, lack of complete pigment removal and a chance of permanent scarring.

Also, certain tattoo pigments, such as Yellow #7, can form toxic products when exposed to laser removal, which can concentrate in the kidneys and liver. Still further, when certain tattoos are removed by laser techniques, complications can arise depending upon the ink or pigmenting material applied during the tattooing process. For example, ignition of traumatically embedded firework debris by use of a lasor during tattoo removal has been observed. See Taylor Charles R.: Laser ignition of traumatically embedded firework debris, Lasers in Surgery and Medicine, 1998/22:157-158.

Studies in guinea pigs have shown imiquimod as an adjuvant to laser tattoo removal, although in this model scarring was also enhanced with imiquimod treatment. See Ramirez M, Magee N, Diven D, Colome-Grimmer M, Motamedi M, Oliveira G, et al. Topical imiquimod as an adjuvant to laser removal of mature tattoos in an animal model. Dermatol Surg 2007 March; 33(3):319-25. Recently, in humans adjuvant imiquomod treatment resulted in a slight improvement in efficacy of laser removal with an enhanced cosmetic outcome. See Elsaie M L, Nouri K, Vejjabhinanta V, Rivas M P, Villafradez-Diaz L M, Martins A, et al. Topical imiquimod in conjunction with Nd:YAG laser for tattoo removal. Lasers Med Sci 2009 November; 24(6):871-5. Although a mechanism for imiquimod has not been elucidated, one of the main hypothesis is that imiquimod enhances the removal of pigment by stimulating macrophages. See Ramirez M, Magee N, Diven D, Colome-Grimmer M, Motamedi M, Oliveira G, et al. Topical imiquimod as an adjuvant to laser removal of mature tattoos in an animal model. Dermatol Surg 2007 March; 33(3):319-25.

Ingenol mebutate, also known as ingenol-3-angelate or PEP005, is currently being developed as a new topical anti-neoplastic drug, which has been shown to be effective and well tolerated in the treatment of actinic keratoses and NMSC in U.S. Pat. No. 6,432,452. (Anderson et al., 2009; Siller et al., 2009; Siller et al., 2010). Preclinical studies have shown the mechanism of action involves the induction of primary necrosis in tumor cells, disruption of tumor vasculature, transient inflammation and recruitment of neutrophils, with treatment resulting in a favorable cosmetic outcome (Challacombe et al., 2006; Li et al., 2010; Ogbourne et al., 2007). Ingenol mebutate has also been suggested for treating photodamaged skin in WO2010/91472, which is incorporated herein by reference in its entirety.

Ingenol mebutate has the following chemical structure, molecular weight and reported properties:

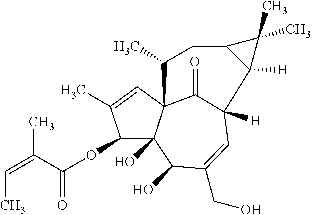

### Product Specification

### Formula: C₂₅H₃₄O₆

### MW: 430.5

### Cas Number: 75567-37-2

Source/Host: Isolated from, e.g., Euphorbia peplus L

### Purity: ≧98% (HPLC)

Appearance: White to off-white solid.
Solubility: Soluble in 100% ethanol, DMSO or dichloromethane.

### Shipping: Ambient

### Short Term Storage: +4° C.

### Long Term Storage: −20° C.

Ingenol mebutate is reported as a specific protein kinase C (PKC) activator, a selective activator of PKC isoforms, like PKCθ in T cells, an antiproliferative and proapoptotic (necrotic) agent, an immunostimulant, an chemotherapeutic, and anticancer compound, and it is an efficacious agent against actinic keratosis. It is also reported that ingenol mebutate is an antileukemic compound.

In view of the above, there are needs for a safe and convenient but effective topical method for removing photodamaged skin, mutated keratinocytes, cutaneous immunosuppressive environments, and/or p53+ patches. Still further, there are needs for a safe and convenient but effective topical method to reduce the number of skin lesions that may evolve from photodamaged skin over time. Still further, there are needs for a safe and convenient but effective topical method to reduce or cure the number of SCC tumors. Still further, there is a need for a safe, convenient and effective noninvasive method to remove tattoos without the disadvantages of the current tattoo removal precodures.

### SUMMARY OF THE INVENTION

The present invention overcomes the above-mentioned disadvantages and shortcomings of the current methods to treat photodamaged skin, to reduce the number of skin lesions that emerge from photodamaged skin over time, and to reduce or cure the nymber of SCC tumors that emerge, and to remove tattoos, through the discovery of new and improved topical methods.

Generally speaking, in one aspect of the present invention, it is directed to new and improved topical field-directed treatment of targeted UV-damaged skin with an effective amount of ingenol mebutate, regardless of the UV-type, e.g., UV-A, UV-B or UV-C, that has caused the skin damage. In another aspect of the invention, it is directed to a topical field-directed treatment of targeted skin lesions that have evolved from photodamaged skin with an effective amount of ingenol mebutate to reduce the number of skin lesions that evolve over time from photodamaged skin. In another aspect of the invention, it is directed to a topical field-directed treatment of targeted SCC tumors with an effective amount of ingenol mebutate to reduce or cure the SCC tumors. In yet another aspect of the present invention, it is directed to a topical field-directed treatment of targeted UV-damaged skin with an effective amount of ingenol mebutate, regardless of UV-type, e.g., UV-A, UV-B or UV-C, to prevent the formation of skin lesions from the photodamaged skin. The present invention is also directed to the topical use of ingenol mebutate in an effective amount to remove UVB-induced p53+ patches, to provide prophylactic utility for ingenol mebutate for removal of photodamaged or UV-damaged skin, and to prevent skin cancer development. The present invention is also directed to the topical use of ingenol mebutate in an effective amount to reverse epidermal thickening and mast cell accumulation caused by UV damage to the skin. It has been discovered that after topical ingenol mebutate treatment, epithelial thickness was similar to unirradiated skin and significantly less than that seen in UVB damaged skin In still another aspect of the present invention, it is directed to a topical field-directed treatment of targeted a tattoo with an effective amount of ingenol mebutate to remove the tattoo from the skin.

By way of example, the present invention is directed to the topical application of about 0.05% ingenol mebutate gel once daily for two consecutive days to targeted UV-damaged or photodamaged skin for removing photodamaged skin, mutated keratinocytes, cutaneous immunosuppressive environments, and/or p53+ patches and/or for reducing the number of skin lesions by up to about 70% or more that subsequently emerged from the photodamaged skin. The present invention also contemplates that topical application of 0.05% ingenol mebutate gel once daily for two consecutive days to targeted UV-damaged or photodamaged skin reduces the number of mutant p53 keratinocyte patches, mutated keratinocytes and/or cutaneous immunosuppressive environments formed by the UV-damaged or photodamaged skin. The field-directed treatment of the targeted UV-damaged or photodamaged skin, in accordance with the present invention, results in epidermal cell death, acute inflammation, recruitment of neutrophils, haemorrhage and eschar formation, all of which resolved over several weeks.

In one exemplary embodiment of the present invention, a hairless SKH-1/hr mouse model is used to demonstrate that field treatment of pre-cancerous chronically UVB-irradiated skin with ingenol mebutate significantly reduced the number of skin lesions that subsequently developed. In addition, the hairless SKH-1/hr mouse model is used to demonstrate that ingenol mebutate treatment also significantly removes and/or reduces the number of mutant p53 patches, mutated keratinocytes and/or cutaneous immunosuppressive environments present in the skin, and, thus, prevents cancer cell formation.

Thus, the present invention illustrates that ingenol mebutate is useful for field-directed treatment for the treatment of UV-damaged skin and the removal of sub-clinical precancerous cells from UV-damaged skin, particularly in patients at high risk of developing non-melanoma skin cancers. The present invention therefore provides a method of prophylactic treatment-prevention of UV-exposed skin, which does not yet exhibit clinical observable UV-related damage, and the treatment-prevention inhibits or reverses the damage to the skin caused by UV.

The present invention is also directed to the topical field treatment of targeted non-melanoma skin cancer lesions with ingenol mebutate to reduce or cure in particular squamos cell carcinoma tumors. More specifically, the present invention is directed to the topical application of up to about 0.25% ingenol mebutate gel to SCC tumors once daily for two days for reducing or curing the number of SCC tumors by about 80%. The field treatment of the SCC tumors in accordance with the present invention, induces hemorrhaging, necrosis, tumor specific antibodies and recruitment of neutrophils to treatment site. Thus, the present invention concerns ingenol mebutate field treatment as a treatment for curing SCC tumors.

The present invention is also directed to the topical field treatment of targeted tattoos with ingenol mebutate to remove the tattoos. More specifically, the present invention is directed to the topical application of about 0.25% ingenol mebutate gel, and more particularly at an ingenol mebutate concentration of from about 0.05% to about 0.25%, and more preferably at an ingenol mebutate concentration of about 0.05%, 0.1% or 0.25%, to a tattoo once daily for two days for removing the tattoo without the disadvantages associated with current tattoo removal procedures.

Thus, in one embodiment of the present invention, it provides for a topical method for treating UV-damaged skin evolving into subclinical actinic keratosis, actinic keratosis, and cancer cells by applying ingenol mebutate.

In another embodiment of the present invention, it provides for a topical method wherein the ingenol mebutate is applied in a concentration of about 0.01% up to about 0.025%.

In still another embodiment of the present invention, it provides for a topical method, wherein ingenol mebutate is administered in the form of a gel.

In yet another embodiment of the present invention, it provides for a topical method, wherein the subject has undergone prior or simultaneous treatment for actinic keratosis or skin cancer.

In still another embodiment of the present invention, it provides for a topical method as above, wherein the individual is treated as a field-directed treatment in areas surrounding the actinic keratosis or skin cancer, such as SCC tumors to treat UV-damaged skin.

In still another embodiment of the present invention, it provides for a topical method, wherein the patient has not been diagnosed with previous skin cancer or actinic keratosis.

In an embodiment of the invention, it provides for a kit of parts comprising a unit treatment for treating photodamaged skin or SCC tumors, or for removing tattoos.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

The foregoing and other objects, advantages and features of the present invention, and the manner in which the same are accomplished, will become more readily apparent upon consideration of the following detailed description of the invention taken in conjunction with the accompanying figure and examples, which illustrate an embodiment, wherein:

FIGS. 1A-1E show the development of UVB-induced epidermal lesions after treatment of UVB-irradiated skin with 0.05% ingenol mebutate gel. SKH1/hr mice were exposed to 1.25 MED of UVB three times per week, for thirty doses. One day after the last irradiation (week 0), mice were treated topically, daily for 2 days with ˜100 μl of 0.05% ingenol mebutate gel or placebo gel (Placebo) over a about 0 cm² area demarcated by a rectangular tattoo on the dorsal (tattoos are shown in FIG. 3). Control mice received the same UVB irradiation, but remained untreated (Control). All mice were examined weekly for the development of UVB-induced lesions over a 21 week period. (a) The mean number of dorsal lesions per mouse per cm2 within the treatment area over time. Five placebo mice were euthanized prior to the end of the study due to excessive tumor burden. Two control mice were euthanized prior to the end of the study due to a groin infection and a swollen abdomen. A third control mouse died for unknown reasons. One control mouse was excluded as lesions inside and outside the treatment area coalesced. After euthanasia the lesion numbers for these mice were no longer included in the means Animal numbers for each group are shown on the figure. At all time points after 12 weeks the ingenol mebutate group showed significantly fewer lesions than the other two groups (p<0.05, Mann Whitney U test). (b) The mean number of dorsal lesions per mouse over time. Mouse numbers and statistics as for a. (c) Mean number of dorsal lesions outside the tattooed areas per mouse. Mouse numbers as for a. (d) Kaplan-Meier curves showing the percentage of mice with a total lesion area within the treatment area of <70 mm². One ingenol mebutate-treated mouse was excluded as the total tumor burden (both inside and outside the treatment area) was excessive and required euthanasia prior to the tumor burden within the treatment area reaching 70 mm². One placebo mouse was excluded for the same reason, and another was excluded as lesions inside and outside the treatment area coalesced. Three control mice were excluded as death was unrelated to tumor burden. The ingenol mebutate group was significantly different from the placebo group (log rank statistic, p<0.001). (e) Kaplan-Meier curves showing the percentage of mice with a total lesion area of <70 mm2. Three control mice were excluded as in d. The ingenol mebutate group was significantly different from the placebo group (log rank statistic, p<0.002).

FIGS. 2A-2C show the growth and size profiles of UVB-induced skin lesions after ingenol mebutate treatment. (a) Growth curves of individual lesions arising in ingenol mebutate and placebo treated, and control mice. The size of individual lesions within the tattoo areas from the mice described in FIG. 1 were measured over time, with Week 0 designated as the week before an individual lesion became apparent. (b) The mean size of the lesions shown in FIG. 2a over time. (c) The size profile of the lesions in ingenol mebutate and placebo treated mice, and control mice at 21 weeks post treatment initiation. Lesions were sorted smallest to largest area.

FIGS. 3A-3B show skin appearance after ingenol mebutate treatment. (a) Photographs of UVB-irradiated skin within the tattooed areas at the indicated times after treatment initiation with 0.05% ingenol mebutate gel. (48 hours represents 24 hours after the second treatment). (b) Photographs of representative mice from ingenol mebutate and placebo treatment, and control groups taken 21 weeks post treatment initiation.

FIGS. 4A-41 show the histology of UVB-irradiated skin treated with 0.05% ingenol mebutate gel. a-i show H&E staining. (a) The skin of naïve mice. (b) The skin of mice irradiated with UVB as described above and treated with placebo gel. (c) UVB-irradiated skin 6 hours after treatment with 0.05% ingenol mebutate gel. (d) UVB-irradiated skin 24 hours after treatment with 0.05% ingenol mebutate gel. (e) UVB-irradiated skin 48 hours after the first treatment with 0.05% ingenol mebutate gel (i.e. 24 hours after the second treatment). (f) High magnification of b. (g) High magnification of c. (h) High magnification of d. (i) High magnification of e. (j-n) show toluidine blue staining of skin as described for a-e. Inserts for k and l show high magnifications of individual toluidine blue staining cells, which are characteristic of mast cells.

FIGS. 5A-5B show ingenol mebutate field treatment reduced the number of mutant p53 patches on SKH1/hr mice that were exposed to 1.25 MED three times per week, for thirty doses. SKH1/hr mice were exposed to 1.25 MED three times per week, for thirty doses. One day after the last UVB irradiation, mice were left untreated (Control), were treated with placebo gel, or were treated with 0.05% ingenol mebutate gel (day 0 and 1). Once the ingenol mebutate-treated areas had healed (4 or 5 weeks after treatment) all mice were euthanized and the epidermis within the treatment areas analyzed for the presence of p53 patches by immunohistochemistry. (a) Representative photographs of a p53 patch in a control animal (arrows). White arrowheads show hair follicles. Bars=100 μm. (b) The number of p53 patches/cm2 in control UVB-irradiated untreated mice (n=20), and UVB-irradiated mice treated with placebo gel (Placebo) (n=17), or ingenol mebutate gel (Ing. meb.) (n=22). The data shown was generated in two independent experiments; each individual experiment showed significant differences (data not shown). Bars represent the mean. (Statistical analysis by Mann Whitney U test).

FIG. 6A is representative of photos taken before (Day 0) and after (Days 1-13) treatment with placebo and 0.01% PEP005 gels.

FIG. 6B shows the number of p53+ patches/cm². Error bars represent the standard error of the mean (SEM).

FIG. 7A is representative photographs before, during and after treatment with 0.05% PEP005 gel.

FIG. 7B shows the number of p53+ patches/cm² within the treatment areas 4-5 weeks after treatment. Control (n=20), placebo gel (n=17), PEP005 gel treatment (n=22). Error bars represent the SEM. ** (p=0.002, Placebo vs PEP005, Mann Whitney U test).

FIGS. 8A-8C show histology of UV damaged skin treated with placebo or 0.05% PEP005 gel.

FIG. 9A shows a number of dorsal lesions per mouse. Placebo vs. PEP005, # p<0.05, *p<0.005, Mann Whitney U tests. One mouse in the PEP005 gel treated group was euthanized due to excessive tumor burden and another due to a groin infection. In the placebo gel treated group 2 mice were euthanized due to infected groins. In the Control group 1 mouse was euthanized due to excessive tumor burden. Averages only included data from living animals.

FIG. 9B shows a reduction in treatment area after 0.05% PEP005 gel treatment. Exclusions as in A plus an additional mouse in the Control group that was euthanized due to groin infection. * p=0.002, Mann Whitney U test.

FIG. 9C shows a number of dorsal lesions within the tattooed area/cm². Exclusions as for B. There was no significant differences between PEP005 and the other groups at weeks 8 and 9.

FIGS. 10A-C are representative photographs from control mice.

FIGS. 10D-F are representative photos of placebo gel-treated mice. Photographs were taken 21 weeks post-treatment.

FIGS. 10H-I are representative photographs of PEP005 gel-treated mice displaying the spectrum of lesions that developed in these mice. G shows a PEP005 gel treated mouse with no lesions. Photographs were taken 21 weeks post-treatment.

FIG. 11A shows a total number of dorsal lesions per mouse over time. Five placebo mice were euthanized prior to the end of the study due to excessive tumor burden. Two control mice were euthanized prior to the end of the study due to groin infection and swollen abdomen. A third control mice died for unknown reasons. After death the lesion numbers for these mice were no longer included in the mean. # p<0.05 and * p<0.001, Mann Whitney U test, PEP005 vs. placebo.

FIG. 11B shows the area of the tattooed square measured 21 weeks after treatment. Exclusions as for A. * denotes p<0.001, Mann Whitney U test.

FIG. 11C shows a number of dorsal lesions within the tattooed area/cm². Exclusions as for FIG. 11A. # indicates p<0.005 and * indicates p<0.001, Mann Whitney U test, PEP005 vs. placebo.

FIG. 11D shows a number of dorsal lesions outside the tattooed square. Exclusions as for FIG. 11A.

FIG. 11E shows Kaplan-Myer curves showing the percentage of mice with a total lesion area of <70 mm². The 3 control mice were excluded as death was unrelated to tumor burden. p=0.002, log rank test, PEP005 vs. placebo.

FIG. 11F shows Kaplan-Myer curves showing the percentage of mice with a total lesion area within the treatment area of <70 mm². One PEP005 gel treated mouse was excluded as excessive total tumor burden required euthanasia prior to the tumor burden within the treatment area reaching 70 mm². One placebo mouse was excluded for the same reason, and another was excluded as lesions inside and outside the treatment area coalesced. Control mouse exclusion as in FIG. 11A. p<0.001, log rank test, PEP005 vs. placebo.

FIG. 12A shows growth curves of individual lesions arising in control mice.

FIG. 12B shows growth curves of individual lesions arising in placebo gel treated mice.

FIG. 12C shows growth curves of individual lesions arising after 0.05% PEP005 gel treatment.

FIG. 12D shows mean growth rates of lesions from FIG. 12A, FIG. 12B and FIG. 12C.

FIGS. 13A-C show bar graphs of the size of individual lesions 21 weeks post-treatment in Control (A), placebo gel treated (B) and 0.05% PEP005 gel treated (C) mice. Lesions were sorted smallest to largest area.

FIG. 13D shows distribution of lesion areas. Tumor lesions from control, placebo gel and PEP005 gel treated mice were grouped into 5 groups according to the specified area ranges. Bars represent the number of lesions within each area range.

FIG. 14A shows H&E staining skin sections.

FIG. 14B shows H&E staining skin sections.

FIG. 14C shows H&E staining skin sections.

FIG. 14D shows H&E staining skin sections.

FIGS. 14E-14H show H&E staining skin sections.

FIG. 15A illustrates shows lines parallel to the treatment rectangle were also tattooed. A schematic diagram of the measurements made after mice were euthanized is shown.

FIGS. 15B-F show the different measurements in control, placebo gel and PEP005 gel treated mice. * indicates p<0.002, Placebo vs. PEP005 Mann Whitney U test.

FIGS. 16A-B show dorsal skin H&E stained sections from outbred male SKH1/hr mice exposed to 1.25 MED three times per week, for thirty doses. One day after irradiation, the mice were treated topically, daily for 2 days (week 0) with ˜100 μl of 0.05% PEP005 gel over an about 10 cm² area demarcated by a tattoo on the dorsal. FIGS. 16A-B also show Skin from control mice fixed in paraformaldehyde.

FIGS. 16C-D show dorsal skin H&E stained sections from outbred male SKH1/hr mice exposed to 1.25 MED three times per week, for thirty doses. One day after irradiation, the mice were treated topically, daily for 2 days (week 0) with ˜100 μl of 0.05% PEP005 gel over an about 10 cm² area demarcated by a tattoo on the dorsal. FIGS. 16C-D also show twenty one weeks after PEP005 gel treatment mice were sacrificed and skin fixed in paraformaldehyde.

FIGS. 16E-F show dorsal skin sections from naïve inbred unirradiated female SKH1/hr mice, fixed in 10% formalin. Bars represent ˜200 μm.

FIG. 17 shows changes in epidermal thickness following 0.05% PEP005 gel field treatment Epidermal thickness within the treatment area.

FIGS. 18A and C are examples of mice pre-treatment.

FIGS. 18B and D are examples of mice at the end of the experiment. Arrows indicate the position of treated lesions.

FIG. 19 shows in vitro dose response for acute cell cytotoxicity of ingenol mebutate.

FIG. 20 shows treatment of T7 tumours in inbred female SKH1/hr mice with 0.1% ingenol.

FIG. 21 shows treatment of T7 tumours in inbred female SKH1/hr mice with 0.1% ingenol mebutate daily for 2 days.

FIG. 22 shows treatment of T7 tumours in inbred female SKH1/hr mice with 0.25% ingenol mebutate daily for 2 days.

FIG. 23 shows treatment of T7 tumours in inbred male SKH1/hr mice with 0.25% ingenol mebutate daily for 2 days.

FIGS. 24A-24D show the effects of gender on ingenol mebutate cure rates in SKH1/hr mice, results from a direct comparative study.

FIG. 25 shows the effect of gender on ingenol mebutate cure rates in SKH1/hr mice, results from two independent studies.

FIG. 26 shows the appearance of ingenol mebutate gel post topical application on T7 tumors.

FIG. 27 shows images of T7 tumour sites post ingenol mebutate treatment.

FIGS. 28A-28D show H&E staining of in vivo T7 tumours treated with 0.25% ingenol mebutate.

FIGS. 29A-29B show primary necrosis induced by ingenol mebutate treatment of T7 tumours.

FIGS. 30A-30L show mitochondrial changes induced in T7 tumours treated topically with ingenol mebutate.

FIGS. 31A-31F show nuclear and cytoplasmic changes induced in T7 tumours grown on SKH1 mice and treated topically with ingenol mebutate.

FIGS. 32A-32B show ingenol mebutate inducing haemorrhage.

FIGS. 33A-33E shows polymorphonuclear leukocytes following topical treatment of T7 tumours with ingenol mebutate.

FIGS. 34A-34B shows the measurement of anti-T7 antibodies following cure of T7 tumours with ingenol mebutate.

FIG. 35 shows cure of primary tumour with ingenol mebutate does not provide protection against subsequent challenge with T7.

FIG. 36 are photographs of mice before and after treatment with placebo, 0.1% or 0.25% ingenol mebutate gel.

FIG. 37 are mice treated with placebo, 0.1% or 0.25% ingenol mebutate gel.

### DETAILED DESCRIPTION OF THE INVENTION

By way of illustrating and providing a more complete appreciation of the present invention and many of the attendant advantages thereof, the following detailed description and examples are given concerning the novel methods.

In general, the present invention provides novel and improved topical field-directed treatment regimens to treat UV-damaged or photodamaged skin to reduce the number of skin lesions that emerged from the UV-damaged or photodamaged skin over time with a pharmaceutical composition containing an effective amount of ingenol mebutate. In accordance with this aspect of the invention, the present invention contemplates the use of topical ingenol mebutate to prophylatically treat UV-damaged skin to regenerate a new layer of epithelial skin that resembles unirradiated-damaged skin. Also, generally speaking, the present invention provides novel and improved field-directed treatment regimens to treat to cure or cure SCC tumors with a pharmaceutical composition containing an effective amount of ingenol mebutate.

More specifically, a novel field-directed treatment of the present invention concerns the topical application of an ingenol mebutate gel formulated with about 0.05% ingenol mebutate, by weight, once a day for two consecutive days to a UV-damaged or photodamaged skin area to treat the photo-damaged skin, to reduce the number of skin lesions that will emerge from the UV-damaged or photodamaged skin area over time, to remove the photodamaged or UV-damaged skin, and/or to prevent the development of cancer cells from the UV-damaged or photodamaged skin area over time. It has been surprisingly found that when this topical field-directed treatment is followed in accordance with the present invention, not only is a reduction of up to about 70% in the number of skin lesions is observed, but there is significant removal of p53+ keratinocytes and a reversal of the UVB-induced epidermal thickening and mast cell accumulation observed, as compared to when such UV-damaged or photodamaged skin area remains untreated. Thus, the present invention provides for the use of ingenol mebutate gel as a prophylatic and/or therapeutic for UV-damaged or photodamaged skin.

In one embodiment, the present invention uses the outbred SKH1/hr model of UV damage to assess the potential use of topical PEP005 as a field therapy for UV-induced photodamage. One to three days after cessation of UV irradiation, PEP005 is applied daily for two days, and 2 or 4 to 5 weeks (depending on PEP005 dose) after cessation of UV irradiation the number of p53+ patches was determined. In this embodiment, it is shown that topical PEP005 is suitable to treat individual UV-induced lesions by 2 consecutive daily applications of PEP005 over the UV-damaged skin prior to skin lesion formation to reduce the number of skin lesions that will emerge from the UV-damaged skin or to treat photodamaged skin.

Also in accordance with the present invention, a novel field-directed treatment of the present invention concerns the topical application of an ingenol mebutate gel formulated with about 0.025% ingenol mebutate, by weight, once a day for two consecutive days to an area of the skin diseased with SCC tumors to reduce or cure the number of SCC tumors in the skin treatment area. It has also been surprisingly found that when this topical field-directed treatment is followed in accordance with the present invention, a reduction or cure of up to about 80% in the number of SCC tumors is observed. Thus, the present invention also provides for the use of an 0.025% ingenol mebutate gel as a therapeutic for to treat SCC tumors.

In accordance with the present invention, the inenol mebutate may be in amorphous or crystalline form either as the free compound or as solvate (for example, of water, i.e. hydrates, or of common organic solvents such as alcohols) and it is intended that both forms are within the scope of the present invention. Methods of solvation are generally known within the art, for example recrystallisation from a given solvent.

Also in accordance with the present invention and used herein, the word “treating” or “treatment” refers to the regression, elimination, partial or full removal or detachment, clearance, reduction in size (e.g. surface area or volume), or otherwise desired decrease in size, number or growth rate of the lesion(s) or tumor(s). Thus, in one or more embodiments of the invention, the use of ingenol mebutate in treating UV-damaged skin or SCC tumors may advantageously promote or improve the rate, degree, extent or time taken for elimination, removal, clearance, reduction in size, or otherwise decrease in size, growth rate or number of skin lesions or SCC tumors on the patient.

While the treatment area to be treated may be of any size (surface area), for example, having a surface area greater than about 500 or even 1000 mm², in certain embodiments of the invention, the treatment area to be treated advantageously has a surface area of about 250 mm² or less. In further embodiments thereof the treatment area has a surface area of about 150 or 100 mm² or less. In still further embodiments, the treatment area has a surface area of about 75 or 50, 25 or 10 mm² or less.

In accordance with the present invention, the ingenol mebutate is administered to the subject in a therapeutically or treatment effective amount. Suitable effective amounts for administration (dosage) and dosing regimens can be determined by the attending physician and may depend on the particular anatomical site or nature, size or number of the lesion(s) being treated, as well as the general age, and health of the subject. Nonetheless, the present invention contemplates topical pharmaceutical gels formulated with 0.05% ingenol mebutate, by weight, for treating the UV-damaged or photodamaged skin and topical pharmaceutical gels formulated with 0.025% ingenol mebutate, by weight, for treating SCC tumors.

The ingenol mebutate may be administered in any suitable form, such as locally, e.g. by topical application to the UV-damaged skin, or to, and/or the area surrounding, the SCC tumor by injection into the tumor. In particular examples of the invention, the ingenol mebutate is administered by topical application to the UV-damaged skin or the SCC tumor.

The method of delivery of the ingenol mebutate may vary, but necessarily involves application of a topical formulation of the invention to and/or in proximity to an area of body surface affected with UV-damage or one or more SCC tumors. For example, a suitable formulation such as cream, aqueous gel, ointment, paste, plaster, or lotion may be spread on the UV-damaged skin or on, and/or around the base of, the SCC tumor or SCC tumors and optionally, gently rubbed in. A solution may be applied in the same ways, but more typically will be applied with a dropper, swab, or the like, and carefully applied to the UV-damaged skin or to and/or around the SCC tumors. Alternatively, the ingenol mebutate may be impregnated into or coated onto an occlusive dressing which is then placed over the affected area. Petrolatum may be spread on the skin surrounding the SCC tumors to protect it from possible irritation during treatment.

The dose regimen will depend on a number of factors that may readily be determined, such as the size of the UV-damaged skin area or SCC tumor(s) and/or number of SCC tumors and the responsiveness to the treatment, but will normally be one dose per day, with a course of treatment lasting from several days to several months, or until the desired result is effected or a significant diminution in the number of the skin lesions evolded from the UV-damaged skin and SCC tumors are achieved. In general, it is contemplated that the formulation will be applied once daily for two days. With a skin patch or occlusive dressing, the device is generally maintained in place on the body surface throughout a drug delivery period, typically in the range of from about 8 hours to about 72 hours, and replaced as necessary.

In a preferred embodiment of the invention the ingenol mebutate is administered, i.e. applied, topically at the site of the UV-damaged skin or the SCC tumor(s), for example, over the whole or partial surface area of the UV-damaged skin or SCC tumor(s). The ingenol mebutate may be topically applied in any suitable form including solutions, emulsions (oil-in-water, water-in-oil, aerosols or foams), ointments, pastes, lotions, powders, paints, gels, hydrogels, hydrocolloids and creams may be prepared so as to contain liposomes, micelles, and/or microspheres. Suitable carriers or additives include mineral oil, propylene glycol, polyoxyethylene, polyoxypropylene, emulsifying wax, sorbitan monostearate, polysorbate 60, cetyl esters wax, cetearyl alcohol, 2-octyldodecanol, cyclodextrin, isopropyl alcohol, ethanol, benzyl alcohol and water. Alternatively, the ingenol mebutate may be presented in the form of an active occlusive dressing, i.e. where the ingenol mebutate is impregnated or coated on a dressing such as bandages, gauzes, tapes, nets, adhesive plaster, films, membranes or patches.

The formulation of compositions and dressings contemplated herein is well known to those skilled in the art, see for example, Remington's Pharmaceutical Sciences, 18.sup.th Edition, Mack Publishing, 1990. Compositions may contain any suitable carriers, diluents or excipients. These include all conventional solvents, dispersion media, fillers, solid carriers, coatings, antifungal and antibacterial agents, viscosity enhancers, film formers, dermal penetration agents, surfactants, isotonic and absorption agents and the like. The carrier for compositions contemplated by the present invention must be pharmaceutically acceptable in the sense of being compatible with the other ingredients of the composition and not injurious to the subject.

Formulations of the invention may optionally contain a pharmaceutically acceptable viscosity enhancer and/or film former. A viscosity enhancer increases the viscosity of the formulation so as to inhibit its spread beyond the site of application. A film former, when it dries, forms a protective film over the site of application. The film inhibits removal of the active ingredient and keeps it in contact with the site being treated. Solutions that dry to form a film are sometimes referred to as paints.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (0/W) emulsions, and include, for example, cetyl alcohol, glyceryl monostearate, lanolin, and stearic acid. Preferred water-soluble ointment bases are prepared from polyethylene glycols of varying molecular weight.

Creams, are also well known in the art, are viscous liquids or semisolid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

As will be appreciated by those working in the field of pharmaceutical formulation, gels are semisolid, suspension-type systems. Single-phase gels contain gelling agents distributed substantially uniformly throughout the carrier liquid, which is typically aqueous, but also, preferably, contain an alcohol such as isopropyl alcohol, and, optionally, an oil.

Lotions, which are preferred for delivery of cosmetic agents, are preparations to be applied to the skin surface without friction, and are typically liquid or semiliquid preparations in which solid particles, including the ingenol mebutate, are present in a water or alcohol base. Lotions are usually suspensions of solids, and preferably, for the present purpose, comprise a liquid oily emulsion of the oil-in-water type. It is generally necessary that the insoluble matter in a lotion be finely divided. Lotions will typically contain suspending agents to produce better dispersions as well as compounds useful for localizing and holding the active agent in contact with the skin.

Pastes are semisolid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

In one embodiment of the invention, the ingenol compound is topically applied in the form of an isopropyl alcohol-based gel. One suitable formulation includes isopropyl alcohol, benzyl alcohol, a cellulose polymer, such as hydroxyethyl cellulose and buffer (e.g. citrate) at a pH<3. In another embodiment of the invention, the ingenol compound can be formulated for topical application in the form of a macrocetyl ether cream, for example containing cetomacrogel emulsifying wax, white soft paraffin and liquid paraffin.

Formulations may also be prepared with liposomes, micelles, and microspheres. Liposomes are microscopic vesicles having a lipid wall comprising a lipid bilayer, and can be used as drug delivery systems herein as well. Generally, liposome formulations are preferred for poorly soluble or insoluble pharmaceutical agents. Liposomal preparations for use in the invention include cationic (positively charged), anionic (negatively charged) and neutral preparations.

Micelles are known in the art to be comprised of surfactant molecules arranged so that their polar headgroups form an outer spherical shell, while the hydrophobic, hydrocarbon chains are oriented towards the center of the sphere, forming a core. Micelles form in an aqueous solution containing surfactant at a high enough concentration so that micelles naturally result. Micelle formulations can be used in conjunction with the present invention either by incorporation into the reservoir of a topical or transdermal delivery system, or into a formulation to be applied to the body surface.

Microspheres, similarly, may be incorporated into the present formulations and drug delivery systems. Like liposomes and micelles, microspheres essentially encapsulate a drug or drug-containing formulation. Microspheres are generally, although not necessarily, formed from synthetic or naturally occurring biocompatible polymers, but may also be comprised of charged lipids such as phospholipids. Preparation of microspheres is well known in the art and described in the pertinent texts and literature.

Examples of compositions of the present invention are described in U.S. Publication Nos. 2009/0292017 and 20100204318, which are incorporated herein by reference in their entireties. Also, the attachment hereto is also incorporated herein by reference in its entirety.

Examples of various embodiments of the present invention will now be further illustrated with reference to the following examples. Thus, the following examples are provided to illustrate the invention, but are not intended to be limiting thereof. Parts and percentages are by weight unless otherwise specified.

### Example 1

### The SKH1/Hr Model

Outbred SKH1/hr mice were obtained from Charles River Laboratories (Wilmington, N.C., USA) and a breeding colony for outbred SKH1/hr mice was established at QIMR. Male SKH1 mice were used for this study. To prevent any fighting and injury (which promotes tumors), mice were kept two per cage, with mice separated by a physical barrier. All animal experiments were approved by the QIMR Animal Ethics Committee.

### UVB Irradiation

Outbred SKH1/hr mice were irradiated 3 times per week for 10-11 weeks under 6×TL-12/40 W fluorescent tubes (Phillips, Amsterdam, The Netherlands) mounted in parallel. The lamps emit 54% UVB (280-315 nm) and 46% UVA (315-400 nm) (Rebel et al., 2001). During the irradiation mice were segregated into individual boxes (11.5×20 cm) with a piece of 0.125 mm cellulose acetate placed over the box to prevent any UVC reaching the mice. The mice were 26 cm below the UV lamps. Under these conditions the mice received 1.25 times the minimal erythemal dose (MED) of UVB at each exposure, with the total UVB dose being 37.5 MED. One MED was defined as the minimal UVB dose, which caused erythema-oedema evident by visual examination. UVB irradiation was ceased for 2-7 days if any of the mice within a cohort showed signs of overt erythema, and was resumed once the overt erythema had resolved. Such cessation was usually only necessary after the initial 2-3 doses of UVB, as mice become resistant to UVB-induced erythema with repeated exposures.

### Immunohistochemical and Histological Examination of p53 Patches

One to three days after discontinuation of UVB irradiation, a ≈4 cm2 dorsal area was treated topically daily for 2 days with 40 μl of ingenol mebutate gel or placebo gel. The treatment area was demarcated by tattoo. An untreated group served as an additional control. Two doses of ingenol mebutate gel were tested: 0.01% and 0.05% (w/v) gel. Once the treated area had healed (two weeks for the 0.01% ingenol mebutate gel treatment and 4-5 weeks for the 0.05% ingenol mebutate gel treatment), the mice were sacrificed for analysis of mutant p53 patches. The treated skin areas were surgically excised and separation of the epidermis from the dermis was achieved by floating the skin, dermis side down, overnight at 4° C. in thermolysin (200 μg/ml, Sigma, St. Louis, Mo., USA) supplemented with CaC^{l2} (1 mM). The epidermis was fixed in 4% formalin for 10 min at room temperature, and was washed in phosphate buffered saline solution (PBS) before being placed in 70% (by volume in water) ethanol and stored at 4° C. Antigen retrieval was performed by placing the epidermal samples in boiling 10 mM citrate buffer (pH 6.0) for 5 min. Endogenous peroxidase activity was inhibited by incubating in 1.5% H₂O₂ in methanol for 20 min. The epidermal samples were blocked for 1 hour in a solution containing 10% normal rabbit serum, 0.2% bovine serum albumin (Sigma) and 0.1% saponin (Sigma) in PBS. The epidermal samples were incubated with a mutant-specific p53 antibody (clone PAB240, NeoMarkers, Fremont, Calif., USA) diluted 1:25 in 10% normal rabbit serum/0.2% BSA/0.1% saponin/PBS overnight at 4° C. Unbound primary antibody was removed by washing samples in PBS/0.5% Tween20. Signal amplification and recognition of the primary antibody was performed by incubating the samples with a biotinylated rabbit anti-mouse IgG1 secondary antibody (RbαM (IgG1)-Biotin (Zymed, Invitrogen, Carlsbad, Calif., USA) diluted 1:50 in PBS/0.2% BSA/0.1% saponin. The samples were washed in PBS to remove any unbound secondary antibody and incubated for 45 min at room temperature with the Streptavidin/HRP complex (DAKO, DakoCytomation, Glostrup, Denmark) diluted 1:200 in PBS. Following 3 washes in PBS, the signal was visualised with DAB (Sigma). The epidermal sheets were then mounted basal side up onto glass slides in Keizer's Glycerine (Merck, Darmstadt, Germany). A grid placed on top of each epidermal sheet preparation was used to score the number of mutant p53 patches in each square using a light microscope with a 10 or 20× objective. A patch was defined as an area of closely associated p53 staining nuclei ≧6 in number.

For histological studies samples were fixed in 10% formalin and processed for paraffin embedding at the indicated times after ingenol mebutate treatment. Paraffin sections were stained with haematoxylin and eosin (H&E), or toluidine blue using standard protocols.

### Ingenol Mebutate Treatment and Lesion Monitoring

One to three days after discontinuation of UVB irradiation, a ˜10 cm2 rectangular area located centrally on the dorsa and previously demarcated by a tattoo were treated topically, daily for 2 days with 100 μl of 0.05% ingenol mebutate gel or 100 μl of placebo gel. An untreated group served as an additional control. The mice were monitored weekly for the development of lesions both within and outside the treatment areas. Lesions arising on the tattooed lines were considered outside the treatment area. The number and size of lesions was monitored over time. All visible lesions were counted. Lesion <2 mm are mostly actinic keratoses, whereas lesions >3 mm are mostly squamous cell carcinomas (de Gruijl, 2008). The mice were euthanized at the end of the study or earlier in cases where total lesion area was excessive or other welfare issues required euthanasia.

Field Treatment of UVB-Damaged Skin with 0.05% Ingenol Mebutate Gel Reduced the Emergence of UV-B-Induced Skin Lesions

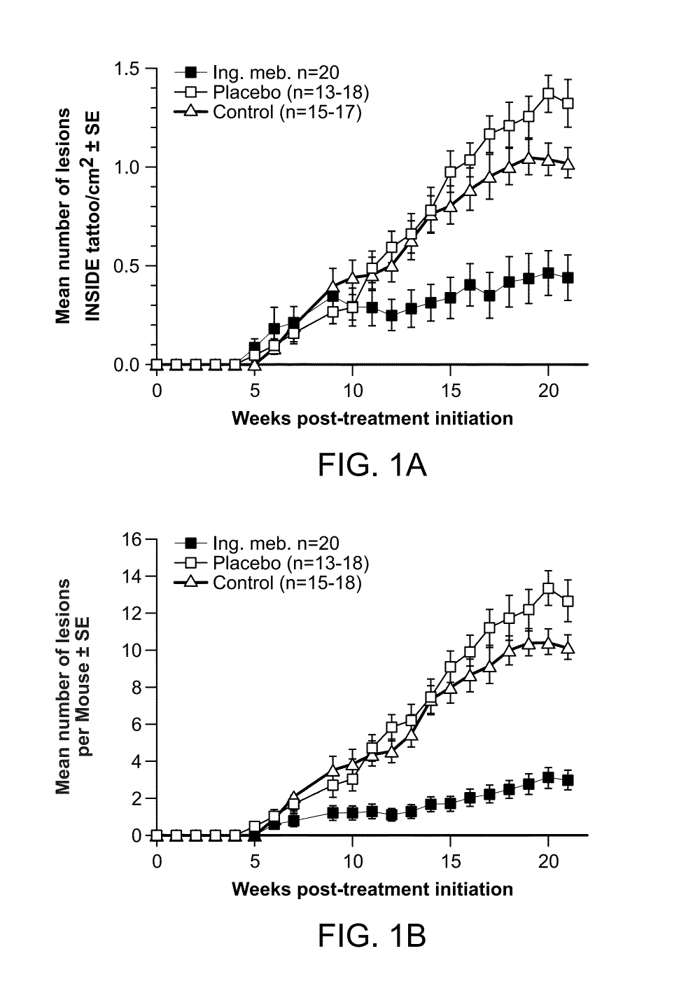

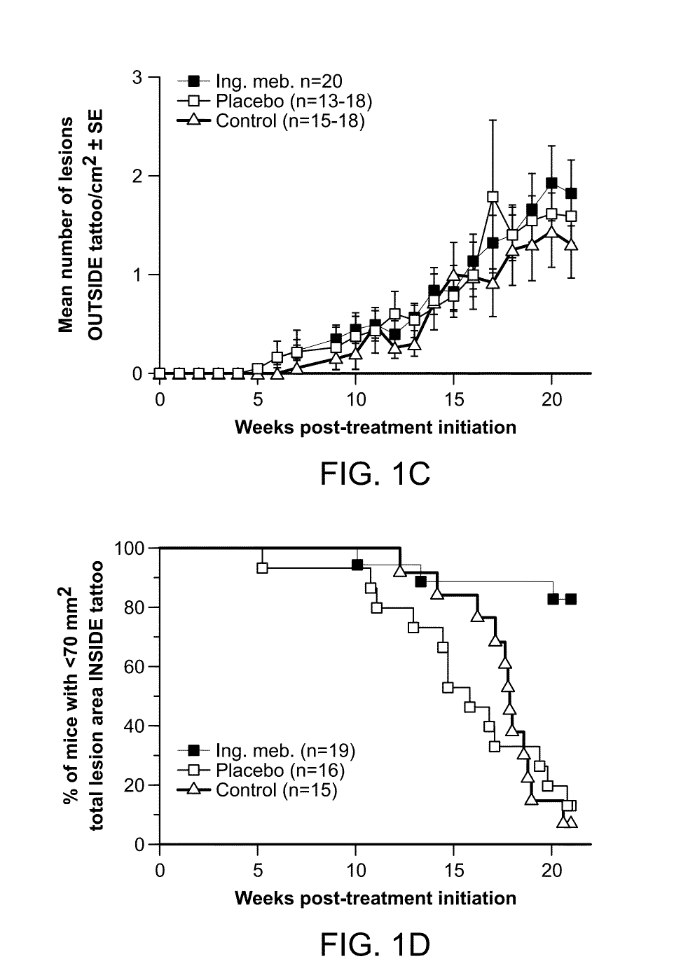

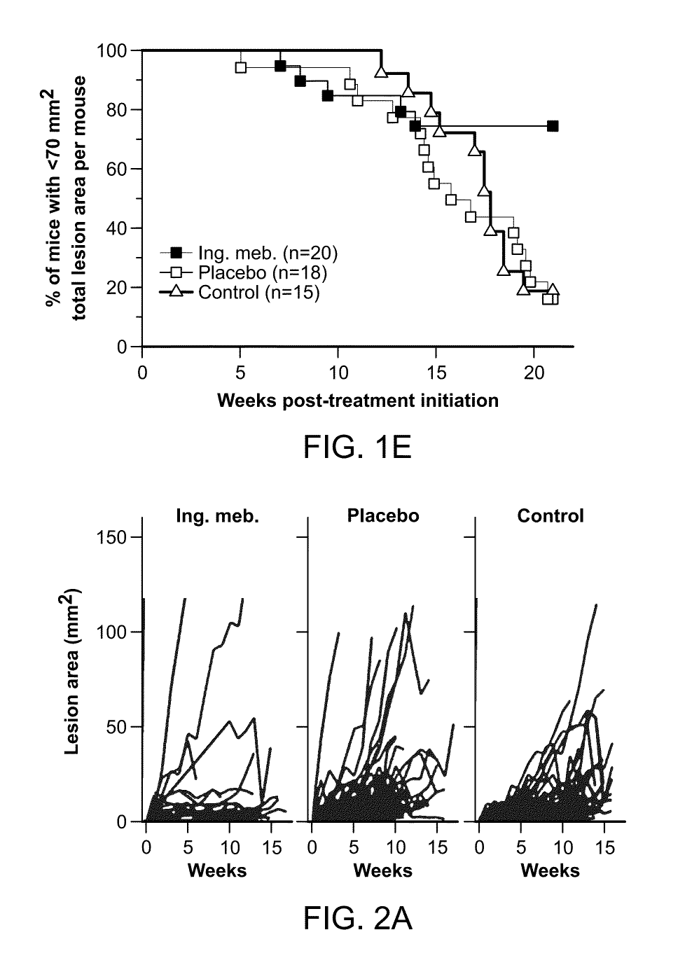

SKH1/hr mice were exposed to 1.25 MED UVB three times per week over 10-11 weeks for a total of 30 doses. One day after the final irradiation dose, the dorsa of the mice were tattooed with rectangles to demarcate the treatment areas. Mice were randomly assigned into 3 groups. The first group was treated topically, daily for 2 days with ˜100 μl of 0.05% ingenol mebutate gel within the tattooed areas (FIG. 1a, Ing. meb.). The second group was treated with placebo gel within the tattooed areas (FIG. 1a, Placebo). The third group remained untreated (FIG. 1a, Control). The mice were examined weekly for 21 weeks for the development of UV-induced skin lesions. Ingenol mebutate gel field treatment resulted in a significant 60-70% reduction in the number of skin lesions per cm2 within the treatment areas that emerged over time compared with placebo treatment and the controls (FIG. 1a). As the treatment areas encompassed much of the UVB-damaged skin of the mouse, this also resulted in a ≈70% reduction in the total number of lesions per mouse (FIG. 1b). The slight increase in the number of lesions seen with placebo gel compared with controls (FIG. 1a, b) may be due to mice scratching the treatment site, with skin wounding or abrasion known to promote epidermal carcinogenesis (Argyris, 1985).

The number of skin lesions outside the treatment areas was not significantly affected (FIG. 1c), suggesting that the ingenol mebutate treatment did not induce effective systemic immunity against keratinocytes over-expressing mutated p53 (Le et al., 2009). This is consistent with the view that mutant p53 patches are not under immune control (de Graaf et al., 2008; Remenyik et al., 2003).

To analyse the effect of 0.05% ingenol mebutate gel treatment on tumor burden, Kaplan-Meier survival curves were generated by analyzing the total lesion area within the treatment area (FIG. 1d) and the total lesion area on the mouse dorsa (FIG. 1e) per mouse over time. For both analyses, an event was assigned when the total lesion area reached 70 mm2. Both analyses showed that 0.05% ingenol mebutate gel treatment led to a significant reduction in the number of mice that had a total lesion area ≧70 mm2 when compared with placebo gel-treated or control mice (FIG. 1d, e). Forty five percent of mice treated with ingenol mebutate actually had no lesions within the treatment areas, whereas placebo and control groups all had at least 5 lesions at week 21 (data not shown). Thus both the number of lesions and the lesion burden (as determined by total lesion area) were significantly reduced by field treatment with 0.05% ingenol mebutate gel.

Growth Rates, Size Distribution and Histology of Lesions Emerging after Ingenol Mebutate were No Different from Those in Control Animals

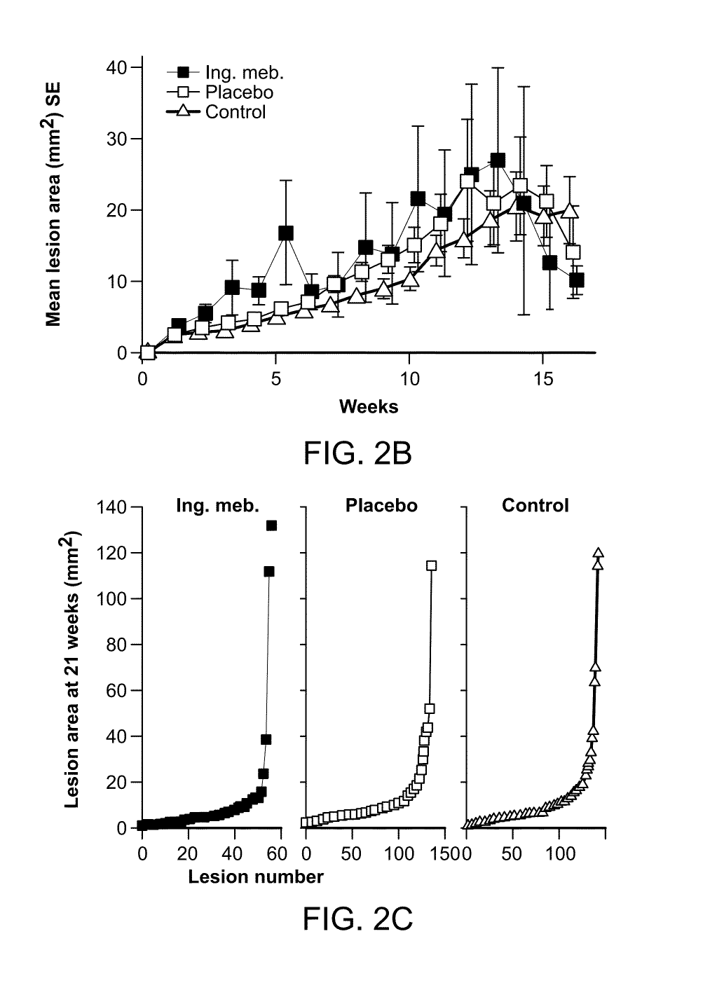

To illustrate that ingenol mebutate treatment did not select for the emergence of more aggressive lesions, the growth kinetics of individual lesions within the treatment areas was examined for each group. The growth of each individual lesion was plotted with Week 0 assigned as the week prior to an individual lesion being identified (irrespective of the time of emergence of the lesion relative to the time of treatment). There was no significant difference in the lesion growth rates between control, placebo and ingenol mebutate gel-treated mice (FIG. 2a, b). (The error bars for the mean area of lesions in the ingenol mebutate treatment group, FIG. 2b, black squares, are larger as the number of lesions was lower).

To determine whether the lesions that arose after ingenol mebutate gel treatment showed any changes in size profile, the size distribution of lesions within the treatment area 21 weeks post-treatment was analyzed. Although more lesions were present in control and placebo gel treated mice compared to ingenol mebutate treated mice, a similar lesion size distribution profile was present in all three groups (FIG. 2c).

Histological analysis of lesions from all three groups showed that lesions were of epidermal origin and resembled the classical picture described previously for lesions in this model (Kligman and Kligman, 1981). No differences in the histology were observed for lesions that emerged after ingenol mebutate treatment and those in the placebo or control groups (data not shown).

These data illustrate that treatment with ingenol mebutate gel did not result in, or select for, the emergence of more aggressive, or larger, or histologically distinct tumors.

Skin Appearance after Ingenol Mebutate Treatment of UVB-Damaged Skin

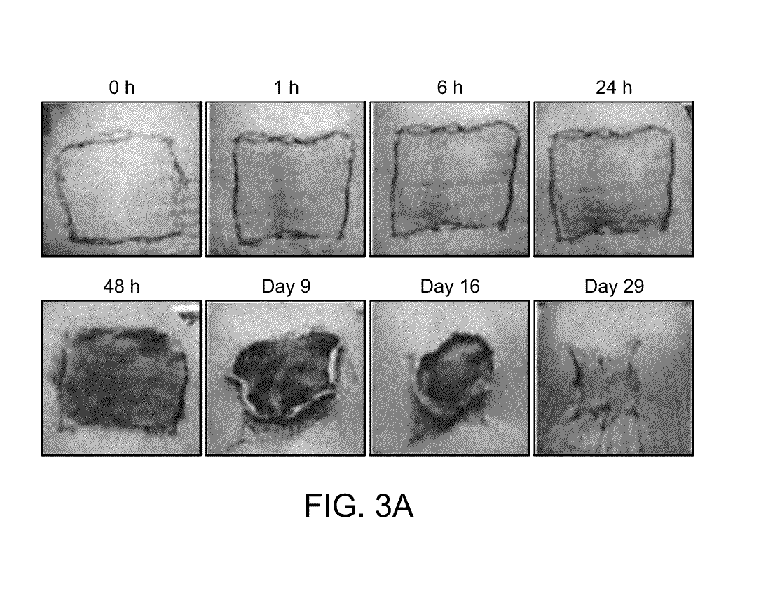

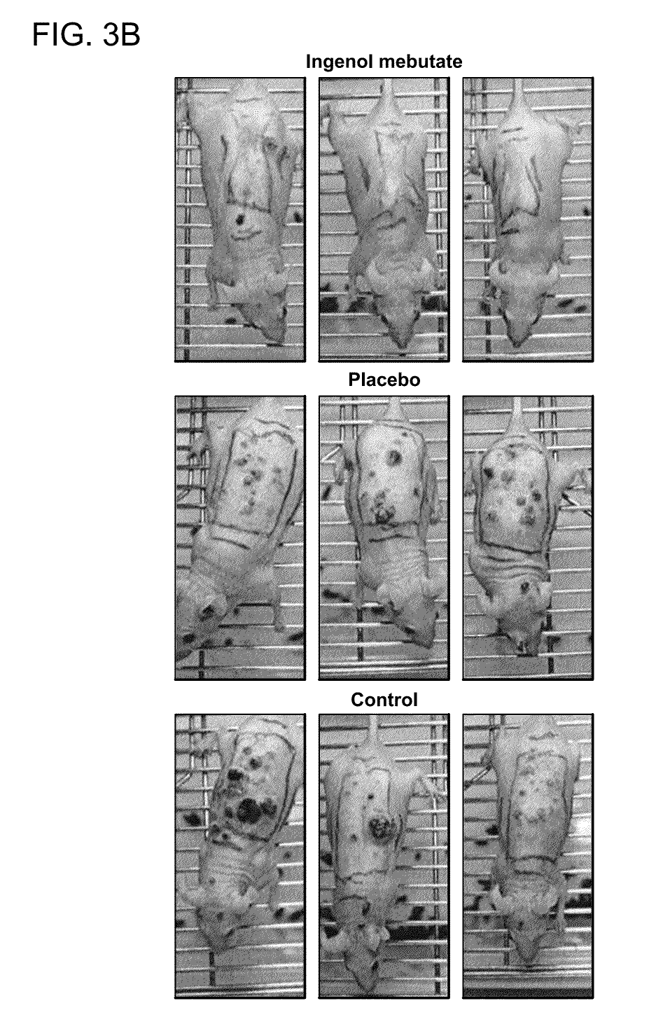

Treatment with about 0.05% ingenol mebutate gel caused reddening of the skin, visible within 1 hour of treatment, with erythema increasing with time (FIG. 3a). By 48 hours (24 hours after the second dose) eschar formation had begun and by day 5-9 it had encompassed the treatment area. The eschar resolved after 3-4 weeks, leaving a pink treatment area (FIG. 3a), which lightened over subsequent weeks.

At week 21 the mean skin area within the treatment area (demarcated by tattoo) had reduced by about 3-fold (FIG. 3). This contraction resulted in skin surrounding the treatment site being pulled towards the treatment site (data not shown). At week 21 after ingenol mebutate treatment, the treated skin felt and appeared largely normal, although slightly darker pink irregular markings remained within the treatment areas (FIG. 3b, Ingenol mebutate).

Skin Histology after Ingenol Mebutate Treatment of UVB-Damaged Skin

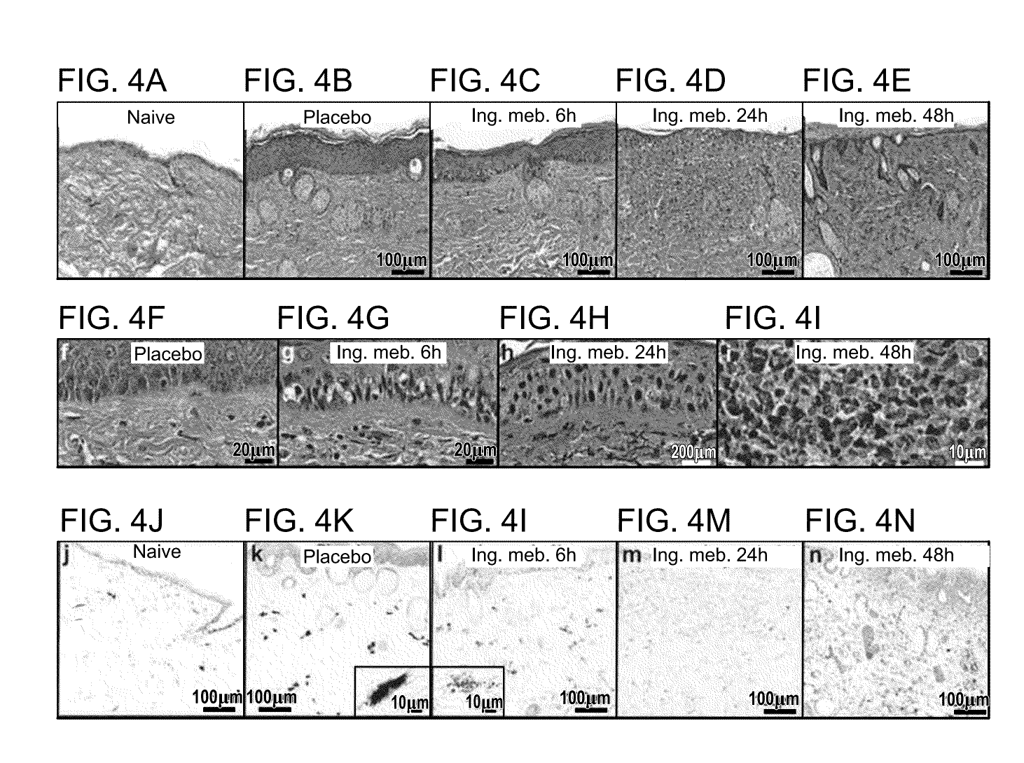

Histology of placebo-treated skin showed the expected epidermal thickening that arises after chronic UVB irradiation (Chaquour et al., 1995); compare normal skin (FIG. 4a) with placebo (FIG. 4b). Six hours after the first treatment with 0.05% ingenol mebutate gel, disruption of the basal epidermis was evident; compare placebo (FIG. 4b) with ingenol mebutate (FIG. 4c). Higher magnifications showed ballooning degeneration of basal keratinocytes; compare placebo (FIG. 4f) with ingenol mebutate (FIG. 4g). By 24 hours post ingenol mebutate treatment, extensive cellular infiltrates (predominant neutrophils) could be seen in the dermis, and extensive areas of epidermal keratinocytes showed hyper eosinophilic staining (FIG. 4d). (This loss of blue basophilic haematoxylin staining in the cytoplasm is usually associated with the loss of ribosomal RNA with a consolidation of cytoplasmic components as the cell collapses). Higher magnification of the epidermis showed keratinocytes with consistent pyknotic nuclei and widespread coagulative necrosis (FIG. 4h). These features are consistent with acute primary necrosis of keratinocytes (Haschek et al., 2009), and illustrated that ingenol mebutate treatment had killed most of the epidermal keratinocytes. At 48 hours (24 hours after second treatment) the epidermis was absent in many places and replaced by an eschar. Healthy (haematoxylin staining) keratinocytes were clearly present in hair follicles and appeared, in several places, to be reconstituting the epidermis (FIG. 4e). This suggests that 48 hours after ingenol mebutate treatment initiation, keratinocyte replication was triggered in the hair follicles and keratinocytes were emerging from the hair follicles to reepithelialize the skin (Lau et al., 2009). Areas containing extensive cellular infiltrates were evident throughout the dermis, and at higher magnification these areas corresponded with pyodermatitis, predominantly composed of red blood cells and degenerating neutrophils (FIG. 4i), consistent with previous observations (Challacombe et al., 2006). At 21 weeks post treatment the lesion-free epidermis in ingenol mebutate treated skin was indistinguishable from normal skin (data not shown).

Toluidine blue staining of skin from Naïve (FIG. 4j) and placebo treated (FIG. 4k) mice showed the increased number of dermal mast cells usually found in chronically UV-irradiated skin in mice and humans (Bosset et al., 2003; Gonzalez et al., 1999; Kligman and Murphy, 1996). Six hours post ingenol mebuated treatment, the mast cell granules, which are stained pink-purple by toluidine blue, appeared more dispersed, suggesting the mast cells were either dying and/or degranulating (FIG. 4l). At 24 hours few toluidine blue staining cells could be seen (FIG. 4m). At 48 hours after treatment initiation, toluidine blue staining cells could again be seen; however, the dominant feature was the extensive red/brown pigment throughout the dermis, indicating widespread haemorrhage (FIG. 4n). At 21 weeks post treatment, the number of dermal toluidine blue staining cells was similar in naïve, placebo and ingenol mebutate treated groups, and no signs of haemorrhage were evident (data not shown).

Field Treatment of UVB-Irradiated Skin with Ingenol Mebutate Reduced the Number of Mutant p53 Patches

Chronic UVB irradiation results in the accumulation of mutant p53 epidermal patches, which are believed to be prerequisite precursors of UVB-induced skin lesions

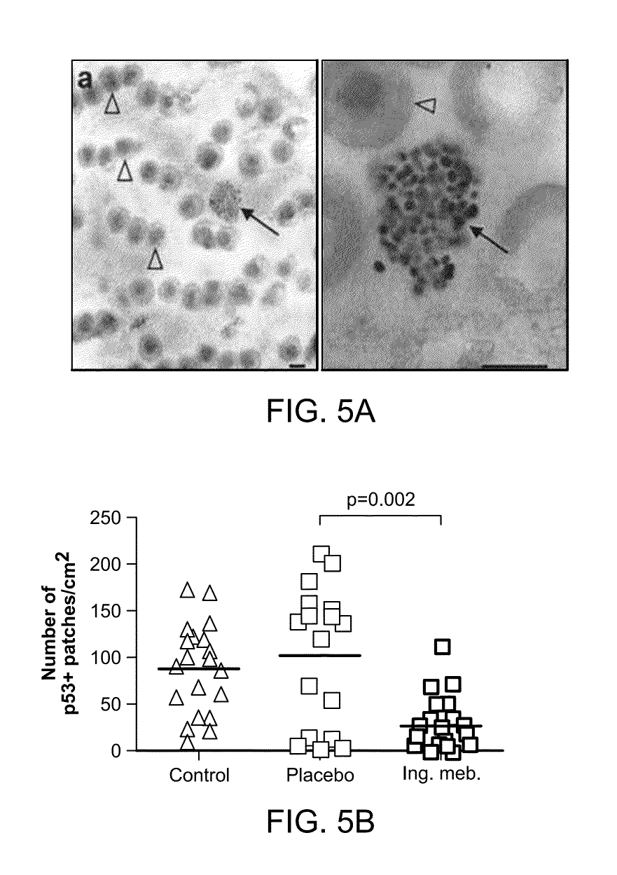

(Berg et al., 1996; Rebel et al., 2005; Rebel et al., 2001). To ascertain whether post-UVB ingenol mebutate field treatment affects p53 patch numbers, SKH1/hr mice were exposed to 1.25 MED three times a week for 11-12 weeks. Mice were then divided into three groups, one was treated on the dorsa with 0.05% ingenol mebutate gel, one was treated with placebo gel, with the third group left untreated (Control). After the healing process was complete in the ingenol mebutate treatment group, the mice from all the groups were euthanized and the epidermis within the treatment areas were analyzed for the presence of p53 patches using immunohistochemistry. An example of a typical mutant p53 patch at low and high magnification is shown in FIG. 5a.

Enumeration of the number of p53 patches showed that chronically irradiated skin in the absence of treatment had 89+10.8 (SE) p53 patches per cm2 (FIG. 5b, Control), whereas unirradiated skin from the underbelly of the same mice showed no detectable p53 patches (data not shown). Placebo gel treatment had no significant effect on p53 patch density (FIG. 5b, Placebo) with 103.3+18.1 (SE) p53 patches/cm2 (FIG. 5b, Placebo). In contrast, treatment with 0.05% ingenol mebutate gel resulted in a significant ≈70% reduction (p=0.002) in the number of p53 patches/cm2 (26.8+6.3 SE) within the treatment areas (FIG. 5b, Ing. meb.). A parallel experiment using a lower concentration of ingenol mebutate (0.01%) had no significant effect on the number of p53 patches (data not shown).

In this Example 1, it shows that field treatment of chronic UVB irradiated skin with 0.05% ingenol mebutate gel results in a about 70% reduction in the number of skin lesions that emerged over time. Ingenol mebutate field treatment causes loss of the epidermis, which appears to be followed by rapid reepithelisation from hair follicles. The treatment also causes a significant reduction in the number of mutant p53 patches in the newly formed epidermis, suggesting removal of replication-competent mutant p53 expressing keratinocytes that give rise to these patches. These experiments highlight the use of ingenol mebutate as a field treatment for chronic UV-damaged or photodamaged skin to prevent the development of NMSC, and in particular SCC.

Ingenol mebutate field treatment may be particularly suitable for individuals, who have a history of UV exposure and are at high risk of developing further NMSC (Bailey et al., 2010; Jonason et al., 1996; Marcil and Stern, 2000). In humans, chronic UV exposure results in accumulation of a substantial burden of premalignant keratinocytes (detectable as p53 patches) from which NMSC are able to develop (Jonason et al., 1996). An individual's risk of developing NMSC depends on a number of environmental and genetic factors (Madan et al., 2010). However, meta-analysis shows that patients who develop one NMSC have an at least 10 fold higher probability of developing another NMSC within 3 years when compared with the general population (Marcil and Stern, 2000). Such patients currently have limited treatment options to reduce their NMSC risk (Bailey et al., 2010), and currently rely on continued monitoring for, and treatment of, newly emerging actinic keratoses and NMSC (Cohen, 2010). Areas of skin that develop actinic keratoses and/or NMSC might be targeted for ingenol mebutate field treatment as they likely identify areas of skin with high malignant potential.

Ingenol mebutate is currently being developed for the treatment of actinic keratosis (Anderson et al., 2009; Siller et al., 2009). In human phase IIa studies 0.05% ingenol mebutate gel applied daily for two days showed a favorable safety profile (Anderson et al., 2009; Siller et al., 2009), (Siller et al., 2010). Skin contractions, like those seen in the mice (FIG. 3b), were not observed. Erythema and eschar formation are also less pronounced (Anderson et al., 2009; Siller et al., 2009; Siller et al., 2010). Mouse and human skin differ in a number of aspects, and so responses to ingenol mebutate are likely to differ. Whether the erythema and eschar formation seen in the mouse is required for the removal of mutant p53 patches is unclear. However, clearance of actinic keratoses in humans (Anderson et al., 2009; Siller et al., 2009) may suggest that topical ingenol mebutate treatment of human skin does result in effective removal of abnormal epidermal keratinocytes. Furthermore, field treatment of up to 100 cm² of human skin with 0.05% ingenol mebutate was generally well tolerated (Schmieder, 2010).

Recently chemical peels are also shown to reduce p53 patch numbers and lesion formation in chronic UVB-irradiated mice (Abdel-Daim et al., 2010). However, this involves 10 treatments (one treatment every 2 weeks), whereas a similar effect was achieved herein with only 2 ingenol mebutate treatments (once daily for two days). In addition, chemical peel treatments continued while lesions and tumors are developing (Abdel-Daim et al., 2010), a scenario unlikely to be countenanced in humans. The chemical peel strategy does however, result in reductions in lesions developing outside the treatment areas (Abdel-Daim et al., 2010), something that is not observed for ingenol mebutate field treatment (FIG. 1c). Treatment of lesions with chemical peels may result in the production of systemic anti-tumor immunity that can regresses distant lesions. It is shown, for instance, that treatment of tumors with ingenol mebutate results in the induction of anti-cancer CD8 T cell immunity, which is capable of regressing distant tumors (Le et al., 2009). Herein ingenol mebutate field treatment is applied several weeks before skin lesions are visible, so significant induction of anti-tumor immunity is unlikely to have occurred. The ingenol mebutate field treatment described herein may expose the immune system to antigens present in p53 patches. However, several groups have shown that p53 patches are not regressed by immune-based mechanisms (de Graaf et al., 2008; Remenyik et al., 2003), so such immunity, even if induced, may have limited effect on p53 patches and subsequent lesion development.

Imiquimod is also used to reduce p53 expression in UV damaged skin in humans although the drug is applied 3 times a week for 4 weeks and the implications for the subsequent development of NMSC and actinic keratoses development is not reported (Smith et al., 2007). Oral retinoids are also reported as being effective in reducing NMSC in high risk patients. However, such treatment may need to be life long and retinoids have the potential to produce adverse effects (Hardin and Mydlarski, 2010; Marquez et al., 2010). It is shown herein that only two treatments over 2 days with ingenol mebutate are sufficient to reduce mutant p53 patch density and lesion formation.

### Example 2

### 1. Summary

The aims of this research were to determine the prophylactic activity of PEP005 in the field treatment of ultraviolet B radiation (UVB)-damaged skin, and the therapeutic activity of PEP005 in the treatment of UVB-induced skin lesions. The outbred SKH1/hr mouse model of UVB-induced p53 mutant (p53+) patches and lesions (previously established at QIMR) was used to address these aims. Topical field treatment with 0.05% PEP005 of SKH1 mice with Photo-damaged skin resulted in significantly fewer p53+ patches/cm², reduction of the UV induced epidermal thickening, and a reduction in cutaneous mast cells. Field treatment of a ˜10 cm² dorsal area of skin, which contained most of the UV damaged skin, with 0.05% PEP005 gel resulted in a ˜70% reduction in the number of UVB-induced skin lesions that emerged after UVB irradiation. These studies support the utility of PEP005 gel for prophylactic field treatment of UVB damaged skin. Topical spot therapy of established UVB-induced lesions with PEP005 gel (0.1-0.25%) resulted in no discernable cures. This model does not appear to be suitable for analyzing the therapeutic activity of PEP005.

### 2. Background

We studied whether topical field treatment of photodamaged skin with low dose (0.01-0.05%, w/v) PEP005 gel leads to the removal of the UVB-damaged keratinocytes and whether such field treatment would also reduce the development of UVB-induced lesions. The hairless, immunocompetent SKH1/hr mouse model has been widely reported in the literature as a model for UVB-induced photodamage, ultimately leading to the development of UVB-induced lesions including actinic keratosis (AK), squamous papillomas and squamous cell carcinoma (SCC) (Cozzi and Suhrbier, 2010). UVB wavelengths emitted by the sun are absorbed by the skin causing erythema, burns, immunosuppression, and DNA damage. The dual ability of UVB to cause malignant transformation of epidermal cells and immunosuppression is thought to contribute to the development of cutaneous malignancies (Ch'ng et al., 2006). Chronic exposure of SKH1/hr mice to UVB leads to the development of “UV-signature” p53 mutations at hotspot codons (Benjamin et al., 2008; de Gruijl and Rebel, 2008). Clonal expansion of keratinocytes with p53 mutations leads to the formation of p53 mutant (p53+) patches/foci, which can be detected by immunohistochemistry (IHC) using an antibody that recognizes the conformation of mutated p53 (Benjamin et al., 2008; de Gruijl and Rebel, 2008). Although some p53+ keratinocytes spontaneously regress in SKH1/hr mice (as they do in humans), others develop into pre-cancerous lesions resembling AKs and can progress to become SCCs (Rebel et al., 2001).

We have established the outbred SKH1/hr model of UVB-induced p53+ mutations at QIMR (Cozzi and Suhrbier, 2010). We have shown that irradiating mice with 1.25 MED three times per week for 8 weeks leads to the formation of p53+ foci, which can be detected by IHC on epidermal sheets. Quantitative analysis of p53+ patches over an area of 5 cm² of epidermis showed ˜300 p53+ patches (Cozzi and Suhrbier, 2010). Two weeks after cessation of UVB irradiation, the size and number of p53+ patches had reduced, but >150 p53+ patches per 5 cm² remained (Cozzi and Suhrbier, 2010). The current study used the outbred SKH1/hr model of UVB damage to assess the potential use of topical PEP005 as a field therapy for UVB-induced photodamage. One to three days after cessation of UVB irradiation, PEP005 was applied daily for two days, and 2 or 4 to 5 weeks (depending on PEP005 dose) after cessation of UVB irradiation the number of p53+ patches was determined.

The current study also determined whether topical PEP005 could be used to treat individual UVB-induced lesions by 2 applications of PEP005 over the lesion.

### 3. Aims

The aims of this Example 2 are to determine the prophylactic and therapeutic potential of PEP005 for treating photodamaged skin and UVB-induced lesions.

For the prophylactic study the following sub-aims were addressed:
1. To determine whether topical field therapy with 0.01% or 0.05% PEP005 gel would lead to the reduction in the number of UVB-induced p53+ patches.
2. To determine whether field treatment with PEP005 gel would result in a reduction in the number of UVB-induced lesions.
3. To examine the histological changes induced by field treatment with PEP005 gel and determine whether neutrophils are recruited to the treatment area.

For the therapeutic study, the aim was to determine whether topical treatment with high dose PEP005 gel (0.1-0.25%) could cure UVB-induced lesions in the SKH1/hr model.

### 4. Materials and Methods

4.1 SKH1/hr model

The SKH1/hr mouse was selected as the model system because: (i) the model generates measurable UV-induced p53 mutations, (ii) some mutant p53 cells are known to progress to AK-like lesions and develop into SCCs, and (iii) this mouse strain is immunocompetent. A breeding program for outbred SKH1 mice has been established at QIMR. Male SKH1 mice were used for this study. To prevent any fighting (which promotes tumors), mice were kept two per cage, with mice separated by a physical bather. Routine animal husbandry was undertaken by the QIMR Bancroft Centre Animal Facility according their standard operating procedures (SOPs). Feed and water was available ad libitum from individual baskets and water bottles attached to the cages.

4.2 PEP005

PEP005 (ingenol 3-angelate, ingenol mebutate) and placebo gel used in this study were provided by Peplin Inc.

0.25% PEP005 gel

0.1% PEP005 gel
Once this stock had finished, subsequent 0.05% PEP005 gel was made by mixing 1:1 volumes of PEP005 (0.1% B/N 033C) with placebo gel

0.01% PEP005 gel

Placebo Gel

4.3 Prophylactic Studies

4.3.1 UVB Regimen

Outbred SKH1/hr mice were irradiated 3 times per week (Monday, Wednesday, and Friday) for 10-11 weeks under 6×TL-12/40 W fluorescent tubes (Phillips) mounted in parallel. During the irradiation mice were segregated into individual boxes (11.5×20 cm) with a piece of 0.125 mm cellulose acetate placed over the box to prevent any UVC reaching the mice. The mice were 26 cm below the UV lamps. Under these conditions the mice received 1.25 times the minimal erythemal dose (MED) of UVB at each exposure, with the total UVB dose being 37.5 MED. One MED was defined as the minimal UVB dose, which caused erythema-edema evident by visual examination. This MED dose had been previously established as suitable for these studies and reported in Cozzi & Suhrbier (2010). UVB-irradiation was ceased for 2-7 days if any of the mice within a cohort showed signs of overt erythema, and was resumed once the overt erythema had resolved. Such cessation was usually only necessary after the initial 2-3 doses of UVB, as mice become resistant to UVB-induced erythema with repeated exposures.

4.3.2 Immunohistochemical Examination of p53+ Patches in UVB-Damaged Skin Following PEP005 Gel Treatment

One to three days after discontinuation of UVB irradiation, an about 4 cm² dorsal area was treated topically daily for 2 days with 40 μl of PEP005 gel or placebo gel. The square treatment area was demarcated by a tattoo. An untreated group served as an additional control. Two doses of PEP005 gel were tested: 0.01% and 0.05% (w/v) gel. Once the treated area had healed (two weeks for the 0.01% PEP005 gel treatment and 4-5 weeks for the 0.05% PEP005 gel treatment), the mice were sacrificed. The treated areas were excised and separation of the epidermis from the dermis was achieved by floating dorsal skin dermis side down overnight at 4° C. in thermolysin (200 μg/ml, Sigma P1512) supplemented with CaCₗ₂ (1 mM). The epidermis was fixed in 4% formalin for 10 min at room temperature, and was washed in phosphate buffered saline solution (PBS) before being placed in 70% v/v ethanol and stored at 4° C. Antigen retrieval was performed by placing the epidermal samples in boiling 10 mM citrate buffer (pH 6.0) for 5 min. Endogenous peroxidase activity was inhibited by incubating in 1.5% H₂O₂ in methanol for 20 min. The epidermal samples were blocked for 1 h in a solution containing 10% normal rabbit serum (NRbS), 0.2% BSA (Faction V, Sigma, Cat No. A9647) and 0.1% saponin (Sigma, Cat No. 47036) in PBS. The epidermal samples were incubated with a mutant-specific p53 antibody (PAB240, Neomerkers, Cat No. MS-104-P) diluted 1:25 in 10% NRbS/0.2% BSA/0.1% saponin/PBS overnight at 4° C. Unbound primary antibody was removed by washing samples in PBS/0.5% Tween 20. Signal amplification and recognition of the primary antibody was performed by incubating the samples with a biotinylated rabbit anti-mouse IgG1 secondary antibody (RbαM (IgG1)-Biotin, Zymed, Cat No. 61-0140) diluted 1:50 in PBS/0.2% BSA/0.1% saponin. The samples were washed in PBS to remove any unbound secondary antibody and incubated for 45 min at room temperature with the Streptavidin/HRP complex (DAKO, Cat No. P0397) diluted 1:200 in PBS. Following 3 washes in PBS, the signal was visualized with DAB (Sigma Cat No. D5905) The epidermal sheets were then mounted basal side up onto glass slides in Keizer's Glycerin (Merk, Cat No. 1.09242.100). A grid placed on top of each epidermal sheet preparation was used to score the number of p53+ patches in each square using a light microscope with a 10 or 20× objective. A patch was defined as an area of closely associated p53 staining nuclei ≧6 in number.

4.3.3 Other Histological Studies on UVB-Damaged Skin Following PEP005 Gel Treatment

Three days after the last dose of UVB irradiation, an about 4 cm² dorsal area was topically treated daily for 2 days with 40 μl of 0.05% PEP005 gel or placebo gel. The treatment area was demarcated by a tattoo. Mice were culled 6 h (6 h) and 24 h (Day 1) after the first dose of 0.05% PEP005 gel and 24 h after the second dose of 0.05% PEP005 gel or placebo gel (Day 2). The samples were fixed in 10% formalin and processed by QIMR histology for paraffin embedding. Paraffin sections were stained with haematoxylin and eosin (H&E), Leder stain or toludine blue by QIMR Histotechnology, in accordance with standard protocols.

4.3.4 Development of UVB-Induced Lesions Following 0.05% PEP005 Gel Treatment

One to three days after discontinuation of UVB irradiation, dorsal areas previously demarcated by a tattoo were treated topically daily for 2 days with 0.05% PEP005 gel or placebo gel. An untreated group served as an additional control. Treatment of two different skin areas was examined, (i) an about 4 cm² area located centrally on the dorsa where the majority of the UVB-induced damaged skin is known to occur, and (ii) an about 10 cm² rectangular area located centrally the dorsa, encompassing the majority of the UVB-damaged skin. Treatment volumes of PEP005 and placebo gels were adjusted according to the treatment area as follows: 40 μl of gel was applied to the about 4 cm² square and 100 μl of gel was applied to the about 10 cm² rectangle. The mice were monitored weekly for the development of lesions both within and outside the treatment area. Lesions arising on the tattooed lines were considered outside the treatment area. The number and size of lesions was monitored over time. At the termination of the study (or earlier in cases where total lesion area was excessive or other welfare issues required euthanasia), the mice were sacrificed and fixed whole in 4-10% paraformaldehyde. Treatment areas from control, placebo gel and PEP005 gel treated mice were excised and paraffin embedded. Standard histology staining was performed by QIMR Histotechnology.

4.4 Therapeutic Treatment of UVB-Induced Lesions with 0.01-0.25% PEP005 Gel

4.4.1 UVB Regimen

Same as 4.3.1 except that UVB-irradiation protocol was continued until visible lesions had formed.

4.4.2 Treatment of UVB-Induced Lesions

Once two or more mice had developed lesions visible by visual examination, the mice were paired and a lesion from each mouse was treated with either 0.1-0.25% (w/v) PEP005 gel placebo gel. The mice were monitored over time for growth of the treated lesions. Mice were euthanized when the total lesion area was deemed excessive on ethical grounds (usually >100 mm²) or the mice showed clear signs of ill health. Mice were also euthanized for animal welfare issues other than tumor burden where necessary.

4.5 Data Analysis

Statistical analysis was performed using Graphpad Prism version 5.03 (Graphpad Software, San Diego Calif. USA, www.graphpad.com) or SPSS for Windows (version 15.0, 2007; SPSS, Chicago, Ill.).

### 5. Results

5.1 Field Treatment of UV-Damaged Skin with 0.01% PEP005 Gel Did not Reduce the Number of p53+ Patches

The aim of the study was to determine whether topical field treatment of UVB-damaged skin in SKH1/hr mice with 0.01% PEP005 gel led to a reduction in the number of p53+ patches. Mice were irradiated with UVB as described in Section 4.3.1. About 4 cm² areas located at the centre of the UVB-damaged skin on the mice dorsa and demarcated by tattoos were treated daily for 2 days with 40 μl of 0.01% PEP005 gel or placebo gel. A control group remained untreated. Two weeks after treatment the mice were sacrificed and the epidermis within the treatment area was analyzed for the presence of p53+ patches as described in Section 4.3.2 above.

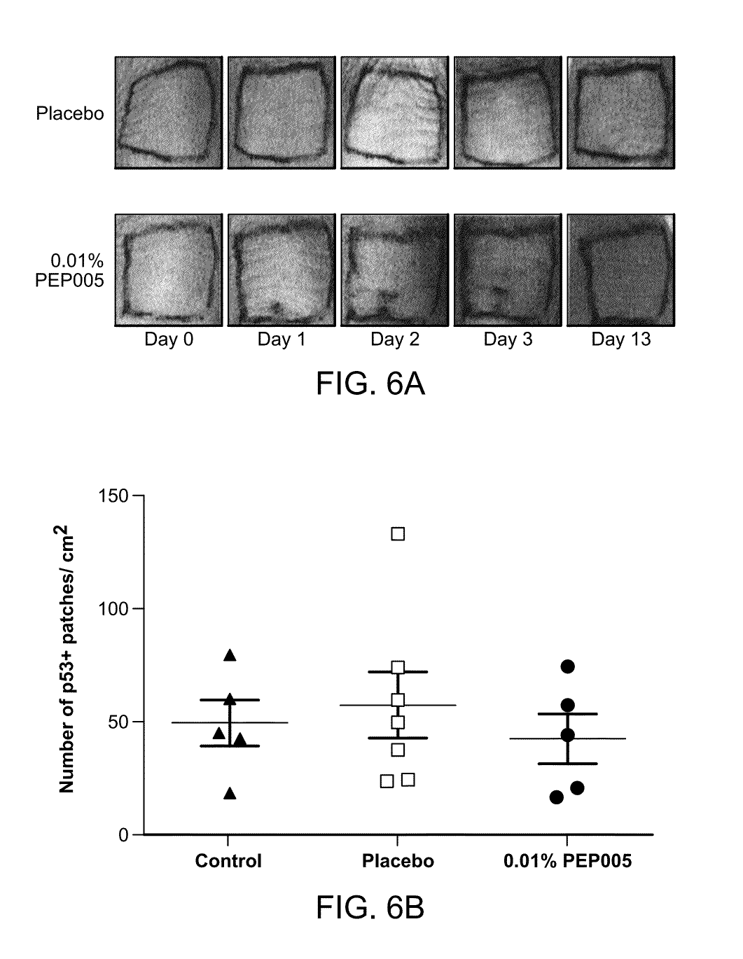

Treatment with 0.01% PEP005 gel induced slight erythema, with flaking and occasionally mild scabbing, which resolved within 2 weeks after treatment, leaving no evidence of scarring or apparent change in the treatment area. See FIG. 6A. Enzymatic separation of the epidermis from the dermis with thermolysin and immunohistochemical analysis of the epidermal sheet showed that treatment with 0.01% PEP005 had no significant effect on the number of p53+ patches/cm² compared to placebo-treated or control mice. See FIG. 6B. This data suggests that topical treatment with 0.01% PEP005 had no beneficial effect on UVB-damaged skin in the SKH1/hr mouse model of photodamage. The treatment dose was subsequently increased to 0.05% PEP005 gel. See Section 5.2 below.

FIGS. 6A and 6B UV show damaged skin treated with 0.01% PEP005 gel SKH1 mice were exposed to 1.25 MED three times per week, for thirty doses. One day after the last UVB irradiation dose, on Day 0 and Day 1, 40 μl 1 of 0.01% PEP005 gel (n=5) or placebo (n=6) gel was applied topically in an about 4 cm² area demarcated by tattoo on the dorsa. Control mice (n=5) remained untreated. On Day 13 (2 weeks after UVB irradiation had ceased) the mice were euthanized and the epidermis within the tattooed area analyzed for p53+ patches.

5.2 Field Treatment of UV-Damaged Skin with 0.05% PEP005 Gel LED to a Reduction in the Number of p53+ Patches

The aim of the study was to determine whether topical field treatment of UVB-damaged skin in SKH1/hr mice with 0.05% PEP005 gel led to a reduction in the number of p53+ patches. Mice were irradiated with UVB as described in Section 4.3.1. An about 4 cm² area on the mice dorsa located at the centre of the UVB-damaged skin and demarcated by tattoo was treated daily for 2 days with 40 μl 1 of 0.05% PEP005 gel or placebo gel. A control group remained untreated. Once the treated area had healed (4-5 weeks after treatment), the mice were sacrificed and the epidermis within the treatment area was analyzed for the presence of p53+ patches as described in Section 4.3.2.

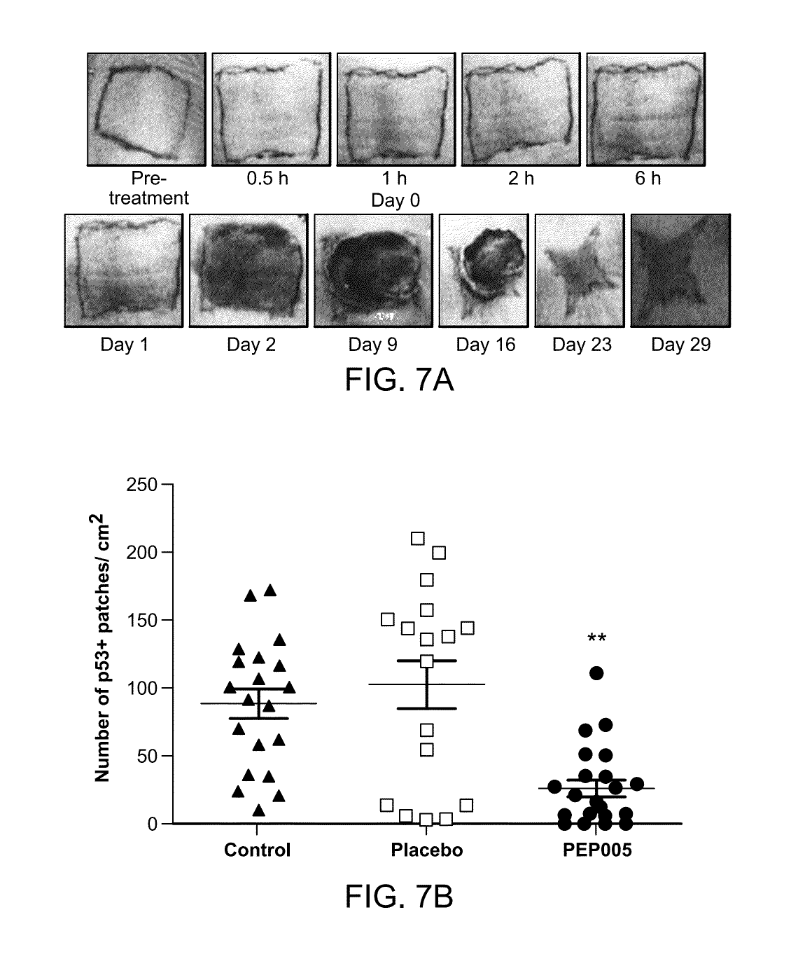

Escalating the treatment dose to 0.05% PEP005 gel caused more of a reaction on the skin. See FIG. 7A. Treatment with a single dose of 0.05% PEP005 gel caused reddening of the skin, visible within 1 hour of treatment, with erythema increasing with time, and hemorrhaging was evident after 24 h. By 24 h after the second dose, an eschar encompassing the whole treatment area had formed which contracted over time and resolved after 4-5 weeks. After this healing process was complete the mean area within the tattoo had reduced by ˜3-fold. See FIG. 7B.

Treatment with 0.05% PEP005 gel resulted in a significant reduction (p=0.002) in the number of p53+ patches/cm² formed within the treatment area. It was harder to separate the epidermis from the dermis in the 0.05% PEP005 gel treatment group and small areas of skin were sometimes lost. However, this was not significant enough to affect the analysis. These data show that topical treatment with 0.05% PEP005 gel removed p53+ patches in this model.

We observed that in the 0.05% PEP005 gel treatment group the tattoo marking was often removed, possibly at sites where the gel had spread over the tattoo. This suggested that PEP005 gel may find a utility as a tattoo removal agent. We further examined this potential new cosmetic indication for PEP005 gel in a small cohort of mice. A report for this study will be submitted as a separate extension to this contract.

FIGS. 7A and 7B show UVB damaged skin treated with 0.05% PEP005 gel SKH1/hr mice were exposed to 1.25 MED three times per week, for thirty doses. One day after the last UVB dose, on Days 0 and 1, 40 μl 1 of 0.05% PEP005 gel or placebo gel was applied topically in the about 4 cm² areas demarcated by tattoo on the mouse dorsa. Control mice remained untreated. Once the treatment area had healed (4-5 weeks after treatment) the mice were euthanized and the treated epidermis analyzed for the presence of p53+ patches by IHC.

5.3 Histological Changes Induced by Treating UVB-Damaged Skin with 0.05% PEP005 Gel

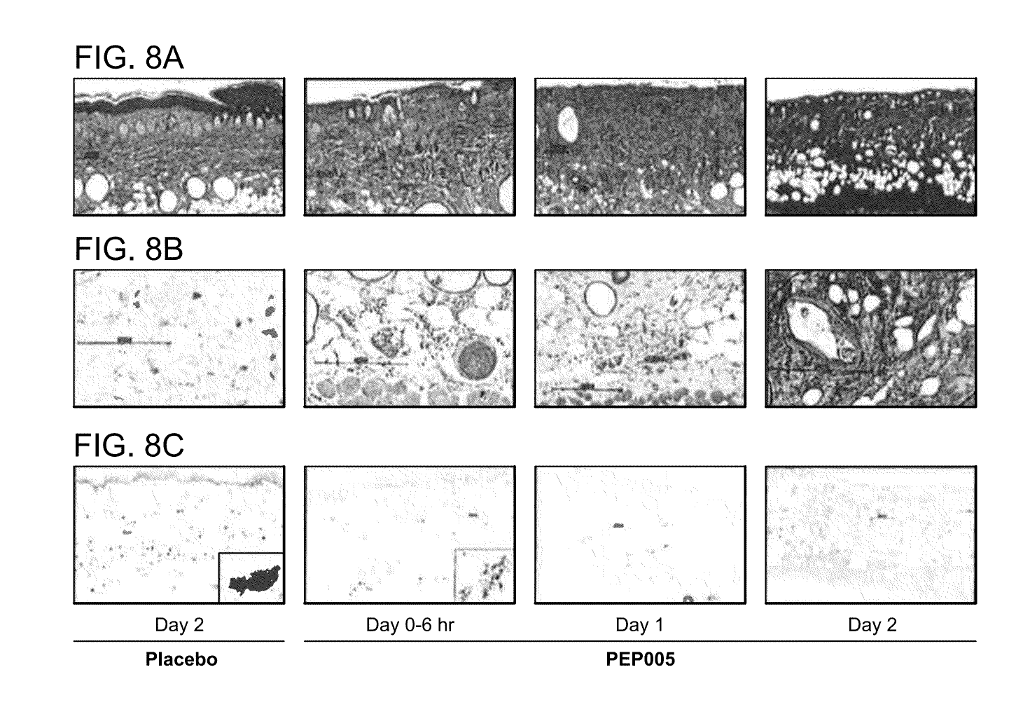

To identify the histological changes after 0.05% PEP005 gel treatment, skin was examined at different times after treatment; 6 h after the first dose was applied (6 h), 24 h after the first dose was applied (Day 1), and 24 h after the second dose was applied (Day 2). Placebo treated mice were treated with equivalent volumes of placebo gel and the skin examined 24 h after the second dose. See FIGS. 8A-8C.

Placebo-treated skin (FIG. 8A) showed the expected epidermal thickening that arises after UVB-irradiation (Chaquour et al., 1995). Six hours after the first treatment with 0.05% PEP005 gel, H&E staining showed a reduction in haematoxylin staining in the epidermal keratinocytes (FIG. 8A, 6 h), consistent with necrosis of keratinocytes. Twenty four hours after treatment with 0.05% PEP005 gel (Day 1), the epidermal layer was no longer clearly visible (FIG. 8A, Day 1). By Day 2 (24 h after the second dose of 0.05% PEP005 gel), extensive hemorrhaging was evident within the treatment area (FIG. 8A).

To assess whether neutrophils were recruited to the site after treatment with 0.05% PEP005 gel, sections were stained with Leder stain that detects the enzyme chloroacetate esterase present in granulocytes. Leder stain stains granulocytes red-pink. See FIG. 8B. Concomitant with the loss of epidermal keratinocytes and epidermal/dermal mast cells and the induction of local hemorrhaging, 0.05% PEP005 gel treatment also led to the recruitment of neutrophils to the treatment site. This was apparent 6 h after the first treatment (FIG. 8B, 6 h) and was extensive 24 h after the second treatment (FIG. 8B, Day 2). The neutrophils stained positively for chloroacetate esterase (FIG. 8B, PEP005) with multi-lobed nuclei apparent when counter stained with haematoxylin. The cells did not stain pink-purple with toluidine blue (FIG. 8C, PEP005) illustrating they were not mast cells.

To determine whether mast cells were present, parallel sections were stained with toluidine blue, which stains mast cells pink-purple. In agreement with the literature (Ch'ng et al., 2006; Brand et al., 2002), UVB-irradiated skin contained abundant mast cells (FIG. 8C, Placebo). Treatment with 0.05% PEP005 gel led to a reduction in the intensity of toluidine blue staining of mast cells by 6 h, indicative of mast cell degranulation and/or death. See FIG. 8C, 6 h. By 24 h, there were very few mast cells in the skin (FIG. 8C, Day 1) and this was also true 24 h after the second dose of 0.05% PEP005 gel (FIG. 8C, Day 2).

FIGS. 8A-8C show histology of UV damaged skin treated with placebo or 0.05% PEP005 gel. SKH1 mice were exposed to 1.25 MED three times per week, for thirty doses. Three days after cessation of irradiation, on Day 0 and Day 1, 40 μl of 0.05% PEP005 gel or placebo gel was applied topically to an about 4 cm² areas demarcated by tattoos on the mouse dorsa. Pictures were taken 6 h (Day 0-6 h) and 24 h after first dose of 0.05% PEP005 gel (Day 1, PEP005) and 24 h after the second 0.05% PE005 gel treatment (Day 2, PEP005). Placebo samples were taken 24 h after the second dose with placebo gel (Day 2, Placebo).

5.4. Field Treatment of an about 4 cm² Area of UVB-Damaged Skin with 0.05% PEP005 Gel Did not Affect the Formation of UVB-Induced Epidermal Lesions

The results in Section 5.1 above suggest that treatment with 0.05% PEP005 gel leads to the reduction in photodamaged keratinocytes, with significantly fewer p53+ patches present after treatment. To determine whether the reduction of UVB-damaged keratinocytes would correlate with reduced formation of UVB-induced lesions, the same experimental design was used as described in Section 5.2. Briefly, SKH1/hr mice were irradiated with 30 doses of 1.25 MED of UVB over 10-11 weeks. The mice were then randomized into three groups and about 4 cm² areas demarcated by a tattoo on the mice dorsa were treated daily for 2 days with 40 μl of 0.05% PEP005 gel or placebo gel. An untreated control group was also included. Following treatment the mice were monitored weekly for the development of lesions both within and outside the treatment area. Both the number and size of the lesions was recorded.

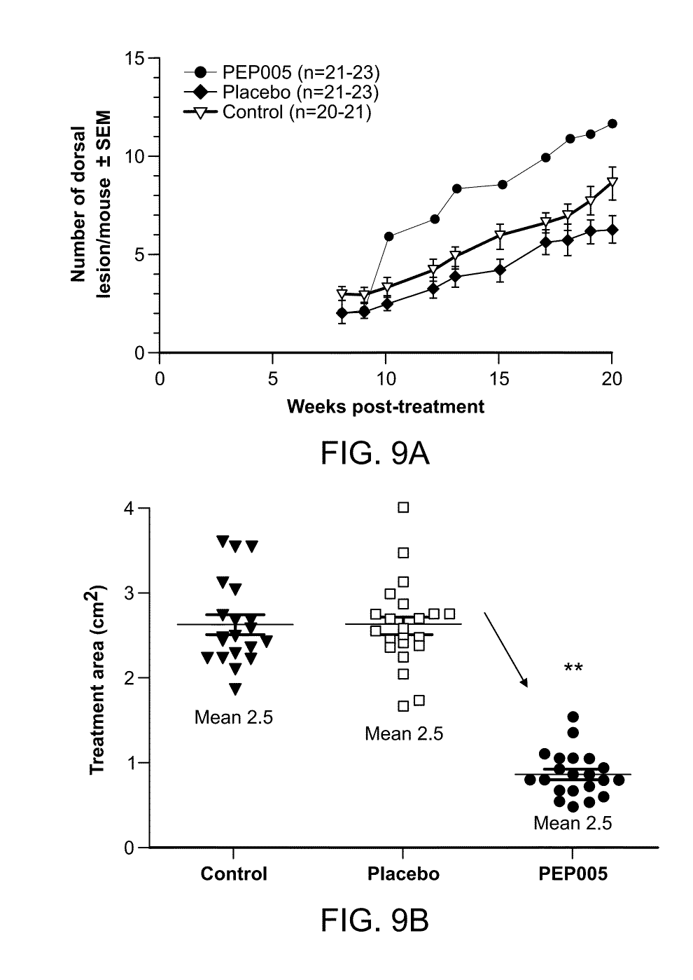

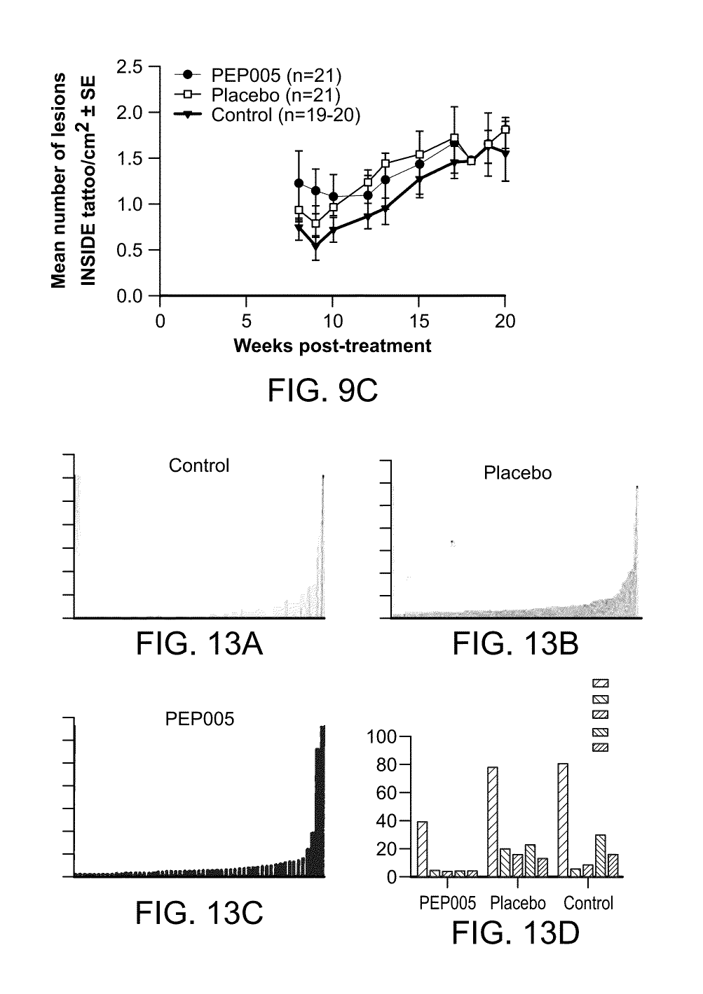

Treatment with 0.05% PEP005 gel led to a small decrease in the total number of UVB-induced lesions per mouse that emerged over time (FIG. 9A) Significance between placebo gel and PEP005 gel treated mice was reached at 13, 15, and 20 weeks post-treatment. See FIG. 9A, *. Control mice also appeared to show a reduced number of lesions when compared to placebo treated mice (FIG. 9A). This may be a consequence of placebo gel treated mice scratching after application of the gel. Scratching is known to increase the number of lesions.

Twenty weeks after treatment with 0.05% PEP005 gel, the area within the tattoo had contracted by ˜3-fold compared to placebo gel and control mice. See FIG. 9B. Although there was a small reduction in the number of lesions per mouse after 0.05% PEP005 gel treatment (FIG. 9A), there was no significant reduction in the number of lesions per cm² within the treatment area (FIG. 9C). The reduction in the number of p53+ lesions within the treatment area thus did not appear to correlate with the reduction in the number of UVB induced lesions.

FIGS. 9A-9C show development of UV-induced lesions after no treatment, placebo treatment or field treatment with 0.05% PEP005 gel in an about 4 cm² area. SKH1/hr mice were exposed to 1.25 MED three times per week, for thirty doses. One to 3 days after the last irradiation, the mice were treated topically, daily for 2 days (week 0) with 40 μl of 0.05% PEP005 gel in an about 4 cm² area demarcated by a tattoo on the dorsa of the mice. Control mice remained untreated. The mice were then examined weekly for the development of UVB-induced lesions.

5.5 Field Treatment of an about 10 cm² Area of UVB-Damaged Skin with 0.05% PEP005 Gel Reduced the Number of UVB-Induced Epidermal Lesions

SKH1/hr mice were exposed to 1.25 MED three times per week for a total of 30 doses. One day after the final irradiation dose, the mice were randomized into 3 groups and about 10 cm² areas demarcated by tattoos on the dorsa were treated topically daily for 2 days with about 100 μl of 0.05% PEP005 gel or placebo gel. Control mice remained untreated. The mice were examined weekly for the development of UV-induced lesions. The number and size of the lesions located within and outside the treatment area were documented.

In addition to the tattooed treatment area, other lines parallel to the treatment rectangle were also tattooed on the mice to track skin movement following 0.05%

PEP005 gel treatment.

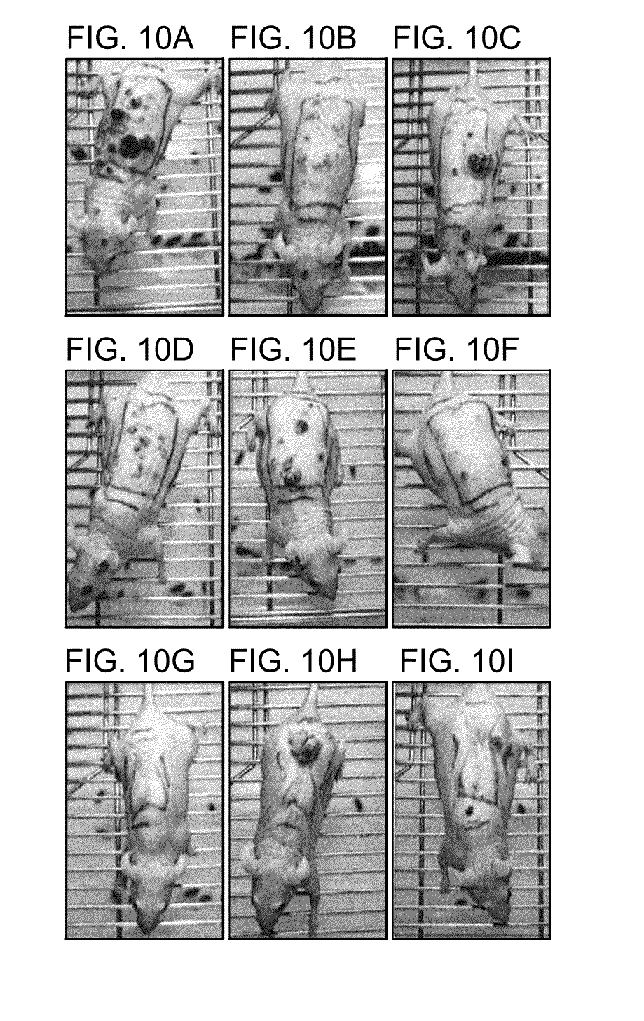

FIGS. 10A-C show mice from the control group (A-C), placebo gel treated group (D-F) and PEP005 gel treated group (G-I), 20 weeks after treatment. The photographs show the spectrum of UVB-induced lesions that developed in untreated, placebo and PEP005 treated mice. They also show the favorable cosmetic outcomes and the treatment site contractions (FIGS. 10G and I), which were also seen after treatment of about 4 cm² areas with PEP005 gel. A slight darker pink residual tainted area remained within the PEP005 treatment area (FIGS. 10G and I), which was not significantly firmer to the touch than normal skin.

FIGS. 10A-I are photographs of mice 21 weeks post-treatment Outbred male SKH1/hr mice were exposed to 1.25 MED three times per week, for thirty doses. One day after of the last irradiation dose, about 10 cm² dorsal areas demarcated by a tattoo were treated topically, daily for 2 days (week 0) with 100 μl of 0.05% PEP005 gel or placebo gel. Control mice remained untreated.

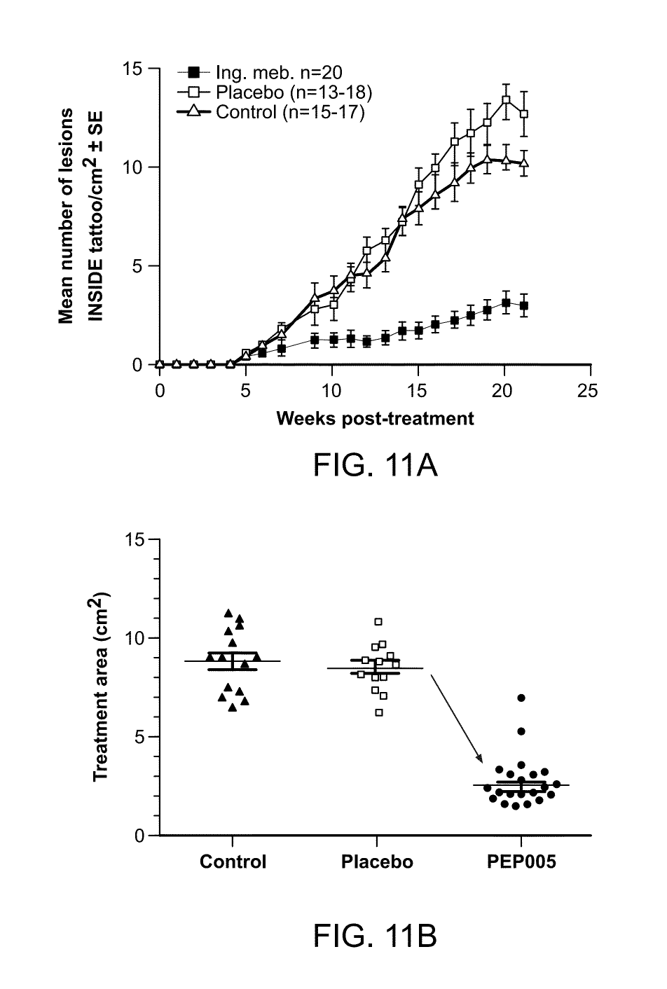

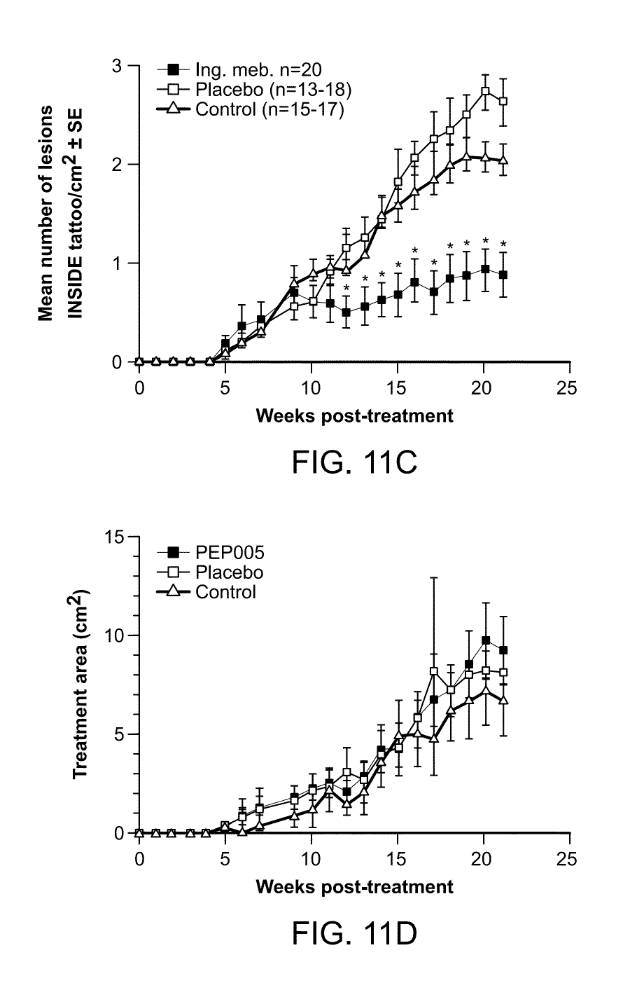

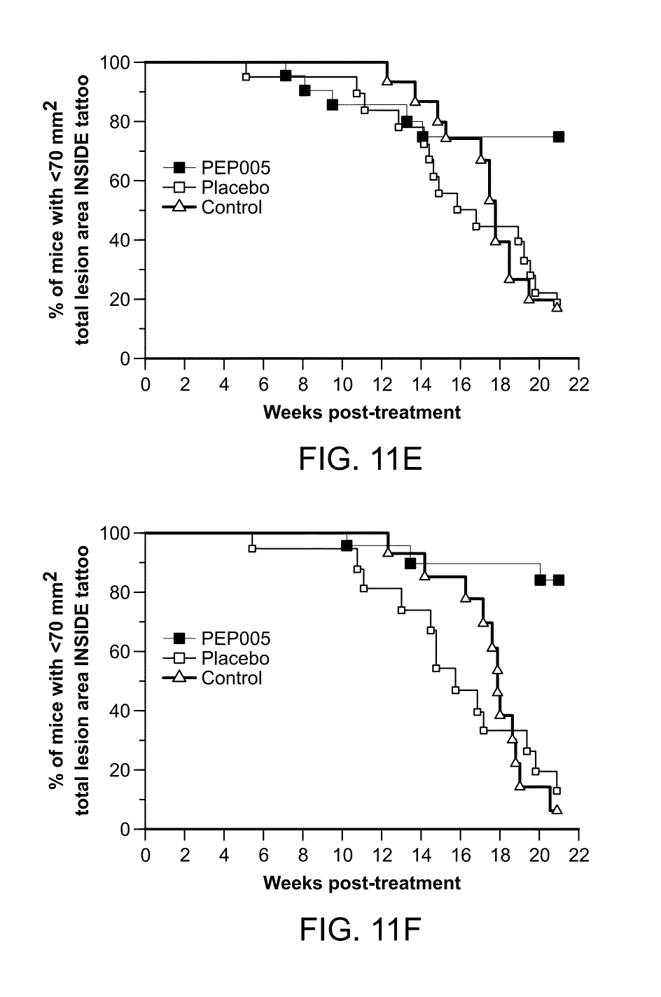

Analysis of lesion numbers in the dorsal area showed that 0.05% PEP005 gel treatment resulted in a significant about 70% reduction in the number of dorsal lesions that developed per mouse. See FIG. 11A. Treatment with 0.05% PEP005 gel again led to a significant contraction (p<0.001) of the treatment area. See FIG. 11B. In contrast with the data shown in FIG. 11C (where the treatment area was about 4 cm²), field treatment of about 10 cm² area with PEP005 gel resulted in a significant reduction in the number of lesions per cm² within the treatment area. See FIG. 11C. The ability of field treatment with PEP005 gel to prevent development of lesions was thus clearly apparent only when a larger area, that represented the majority of the UVB damaged skin, was treated.

To determine whether treatment of UVB-damaged skin with 0.05% PEP005 gel had an effect on lesion development outside the treatment area, the number of lesions outside the tattooed area was enumerated. Treatment with 0.05% PEP005 gel had no effect on the number of lesions that developed outside the treatment area (FIG. 11D), suggesting that there was no systemic anti-cancer activity associated with PEP005 field treatment in this model.

The results described above illustrated that when the majority of photodamaged skin was treated with 0.05% PEP005 gel, a clearly apparent reduction in the number of skin lesions was observed, with both the total number of lesions per mouse significantly reduced (FIG. 11A) and the number of lesions per cm² within the treatment area significantly reduced. See FIG. 11C. To determine whether 0.05% PEP005 gel treatment had an effect on tumor burden, Kaplan-Myer survival curves were generated by analyzing the total dorsal lesion area per mouse (FIG. 11E) and the total lesion area within the treatment area over time. See FIG. 11F. For both analyzes, an event was assigned when the combined lesion area of a mouse reached 70 mm². Both analyzes showed that 0.05% PEP005 gel treatment led to a significant reduction in the number of mice that had a cumulative lesion area 70 mm² when compared with placebo gel-treated or control mice. Thus both the number of lesions (FIGS. 11A, 11C) and the tumor burden (as determined by tumor area) (FIGS. 11, 11F) was significantly reduced by field treatment with 0.05% PEP005 gel.

FIGS. 11A-F show the development of UV-induced epidermal lesions after no treatment, placebo treatment, or field treatment with 0.05% PEP005 gel in an about 10 cm² area. SKH1/hr mice were exposed to 1.25 MED three times per week, for thirty doses. One day after the last irradiation, mice were treated topically, daily for 2 days (week 0) with ˜100 μl 1 of 0.05% PEP005 gel or placebo gel over an about 10 cm² area demarcated by a tattoo on the dorsa. Control mice remained untreated. The mice were examined weekly for the development of UV-induced lesions.

5.6 Lesions Arising after PEP005 Gel Field Treatment Show No Difference in Growth Kinetics.

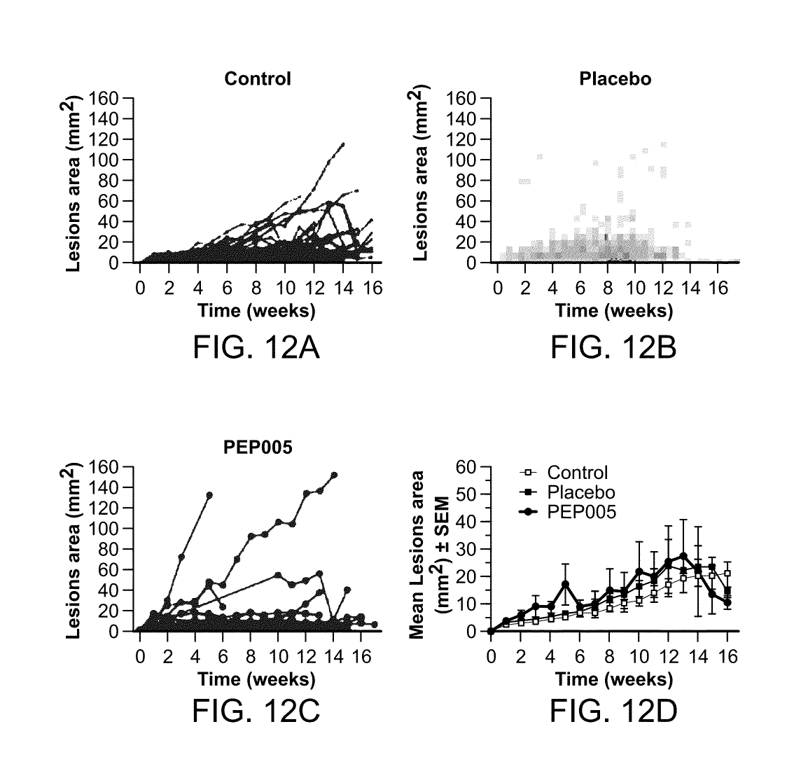

To determine whether there was any difference between the growth kinetics of lesions formed between treatment groups, we examined the growth rates of individual lesions that developed within the treatment areas. Week 0 was assigned as the week prior to an individual lesion being identified. Thus all the individual lesions' growth curves start at week 0, irrespective of the time of treatment. The growth curves in FIG. 12 represent the growth of individual lesions from control mice (A), placebo gel treated mice (B), and 0.05% PEP005 gel treated mice (C). The mean lesion growth for each group is shown in FIG. 12D. There was no significant difference in lesion growth between control, placebo and PEP005 gel treated mice. These data show that the tumors that emerged after PEP005 gel treatment did not grow faster than those in control or placebo-treated mice, suggesting that field treatment with PEP005 gel did not resulted in, or select for, the emergence of more aggressive tumors.

FIGS. 12-A-D show growth curves of UVB-induced lesions Outbred male SKH1/hr mice were exposed to 1.25 MED three times per week, for thirty doses. One day after of the last irradiation dose, the mice were treated topically, daily for 2 days with ˜100 μl 1 of 0.05% PEP005 gel or placebo gel over an about 10 cm² area demarcated by a tattoo on the dorsa. Control mice remained untreated. The mice were examined weekly for the development of UV-induced lesions. Week 0 was assigned as the week prior to an individual lesion being identified. Thus all the individual lesions' growth curves start at week 0, irrespective of the time of treatment of the mouse. All the lesions shown grew within the tattooed areas.

5.7 Lesions Arising after PEP005 Gel Field Treatment Showed Similar Size Distribution to Placebo-Treated and Control Mice

To determine whether the lesions that arose after PEP005 gel treatment showed any changes in size profile, the size distribution of lesions within the treatment area 21 weeks post-treatment was analyzed. Bar graphs show the area (mm2) of individual lesions 21 weeks post treatment in control mice (FIG. 13A), placebo gel treated mice (FIG. 13B) and PEP005 gel treated mice (FIG. 13A). Although a significantly higher number of lesions were examined in control and placebo gel treated mice compared to 0.05% PEP005 gel treated mice, a similar size distribution of lesions was present in all three groups. Furthermore, when lesions were grouped into 5 categories based on size ranges, there was a similar distribution profile. See FIG. 13D. This data illustrated that in this setting lesions arising after PEP005 gel treatment were similar in size to those arising in control and placebo gel treated mice.

FIGS. 13A-D show size distributions of lesions at week 21 post treatment. Outbred male SKH1/hr mice were exposed to 1.25 MED three times per week, for thirty doses. One day after the last irradiation does, the mice were treated topically, daily for 2 days with ˜100 ml of 0.05% PEP 5 gel or placebo gel over an about 10 cm² area demarcated by a tattoo on the dorsa. Control mice remained untreated. The mice were examined weekly for the development of UV-induced lesions.

5.8 Histological Analysis of UVB-Induced Lesions and Skin Treated with 0.05% PEP005 Gel

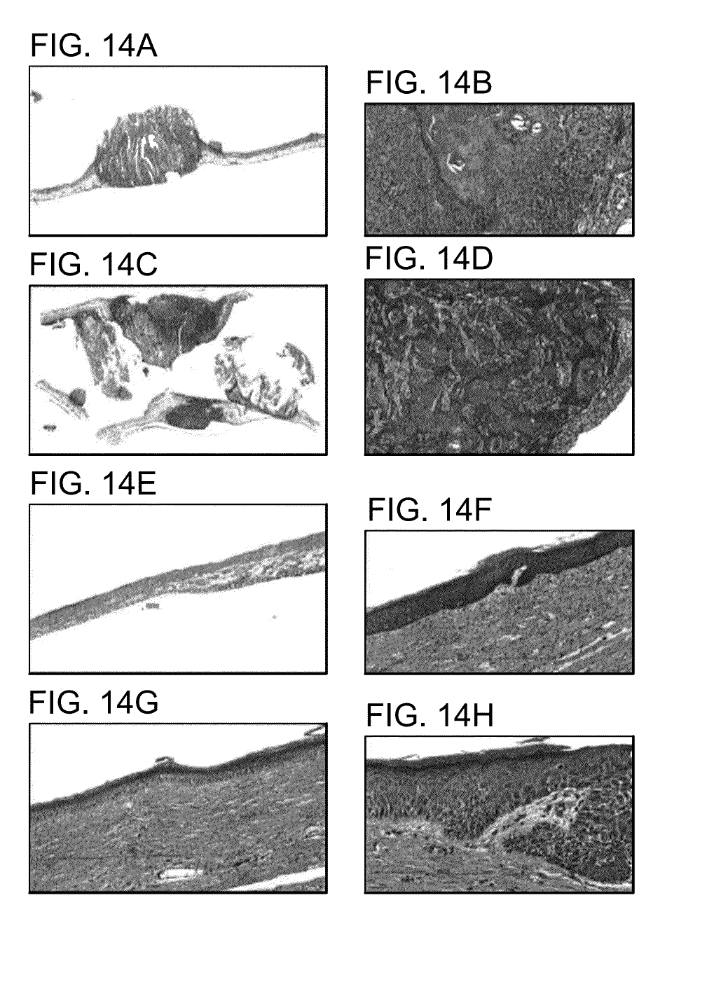

H&E staining of representative control (FIGS. 14A-D) and placebo gel treated mice (FIGS. 14C-D) confirmed that UVB irradiation led to the formation of lesions that were of epidermal origin. The presence of multiple lesions within the one area was evident at lower magnification (FIGS. 14A and C). Higher magnification (FIGS. 14B and D) shows the presence of abundant keratin swirls between the cells. Three mice per group were analyzed.

Low magnification images of 0.05% PEP005 gel treated skin from two mice with no visible lesions showed that the skin was also free of histologically observable lesions (FIGS. 14 E and G). Most of the skin appeared to have reverted back to resemble unirradiated naïve skin (FIG. 14G) although the occasional area of thickened epidermis was still present (FIGS. 14F and H). Three mice per group were analyzed.

FIGS. 14A-D show H&E staining skin sections. Outbred male SKH1/hr mice were exposed to 1.25 MED three times per week, for thirty doses. One day after the last irradiation dose, the mice were treated topically, daily for 2 days over an about 10 cm² area with about 100 μl of 0.05% PEP005 gel or placebo gel. Skin from untreated control mouse at 21 weeks. See FIGS. 14A and 14B. Skin from placebo gel treated mouse analyzed 17 weeks after treatment. See FIGS. 14C and 14D. Skin from PEP005 gel treated mouse 21 weeks after treatment. Bars represent about 500 μm.

5.9 Skin Contraction Following Field Treatment with 0.05% PEP005 Gel

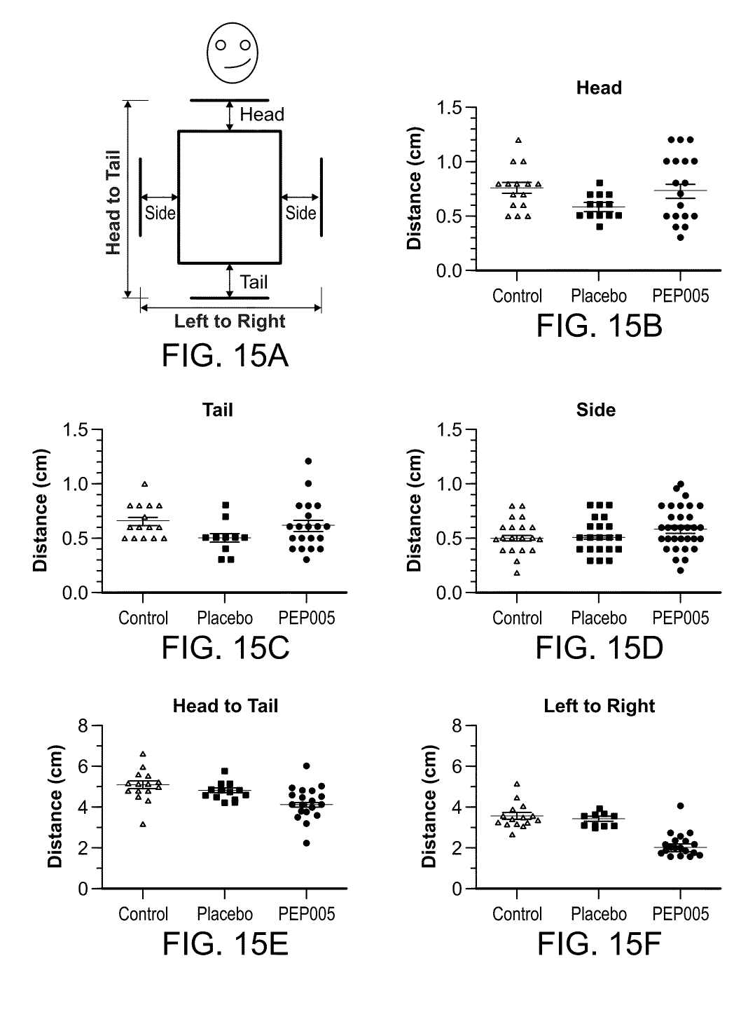

In the current study treatment of UVB-damaged skin with 0.05% PEP005 gel resulted in apparent chemoablation of the epithelial skin layer. A neutrophil-mediated response was apparent within 24 h of treatment (FIG. 8), followed by the formation of a scab 24 h after the second dose of PEP005 gel (FIG. 2A). At 20-21 weeks after 0.05% PEP005 gel treatment, skin had contracted, with a reduction in area of ˜3 fold (FIGS. 9B and 11B). To better understand how this skin contraction affected neighboring skin, lines were tattooed parallel to the tattooed about 10 cm² treatment rectangles. A schematic diagram is shown in FIG. 15A. When the mice were euthanized (21 weeks after treatment) measurements of the distances between the lines in each group was recorded. See FIGS. 15B-F. A significant reduction (p<0.002) was only seen in the head to tail and left to right measurements. See FIGS. 15E and F. This suggests that the contraction of skin after PEP005 gel treatment resulted in surrounding skin being pulled towards the treatment area.

FIGS. 15A-F show skin contraction after field treatment with PEP005. Outbred male SKH1/hr mice were exposed to 1.25 MED three times per week, for thirty doses. One day after the last irradiation dose, the mice were treated topically, daily for 2 days with ˜100 μl 1 of 0.05% PEP005 gel or placebo gel over an about 10 cm² area demarcated by tattoos on the dorsa. Control mice remained untreated. The following mice were excluded from the analysis as they did not complete the study. Five placebo mice were euthanized prior to the end of the study due to excessive tumor burden. Two control mice were euthanized prior to the end of the study due to groin infection and swollen abdomen. A third control mice died for unknown reasons.

5.10 Morphological Changes Induced by UVB-Damage and their Reversal After Field Treatment with 0.05% PEP005 Gel

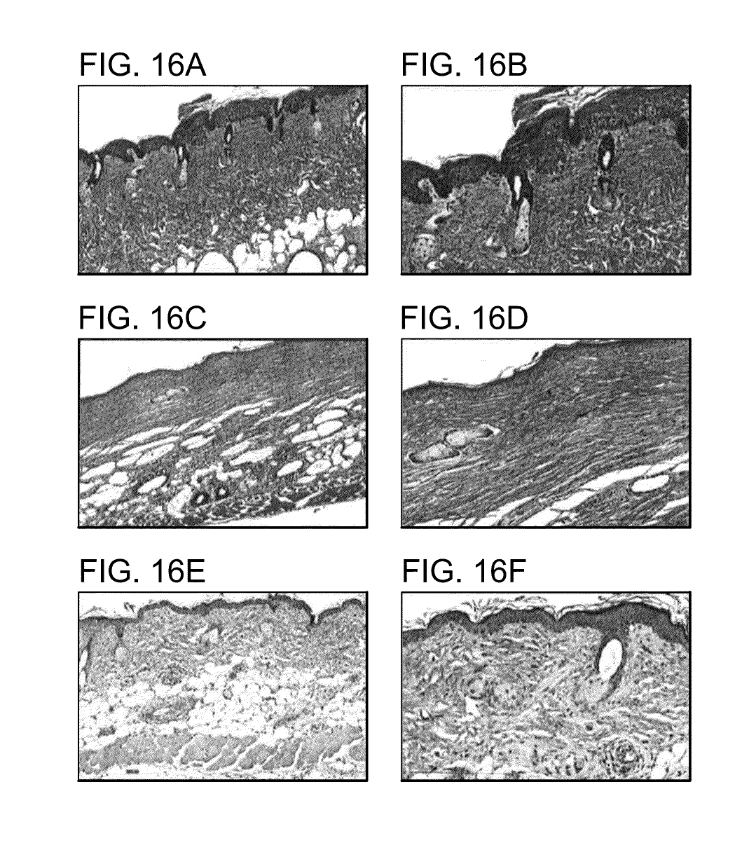

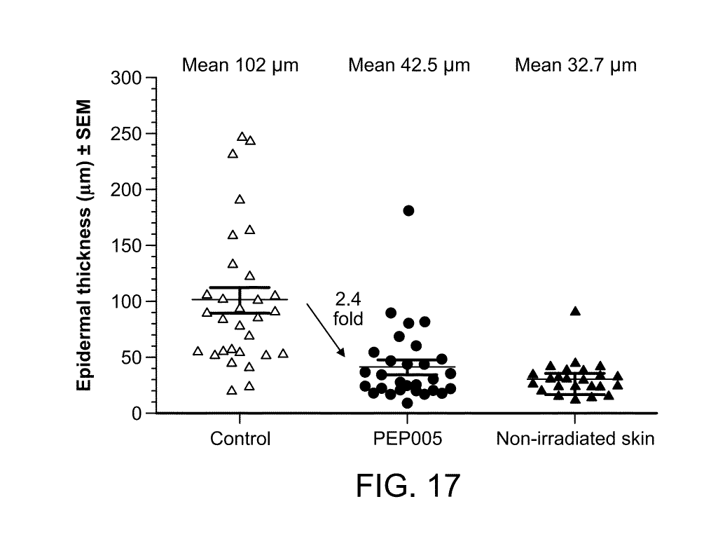

Visual examination showed that by 21 weeks after 0.05% PEP005 gel treatment, the skin is healed with a favorable cosmetic outcome, in many cases devoid of UVB-induced lesions (FIGS. 10G-I). Histological analyzes by H&E staining of dorsal skin from control mice shows that UVB-induced epidermal thickening is maintained 21 weeks after irradiation cessation. See FIGS. 16A-B. Re-epithelisation following 0.05% PEP005 gel treatments resulted in an epithelial layer with a thickness (FIGS. 16C-D) similar to that found in unirradiated naïve mice (FIGS. 16E-F). Quantitative analysis of the thickness of the epithelial layer in histological sections of skin from UVB-irradiated control mice, 0.05% PEP005 gel treated mice and non-irradiated mice, showed that the epidermis from UVB-irradiated control mice was thicker than that in UVB-irradiated and 0.05% PEP005 gel treated mice. See FIG. 17. The regenerated epidermis in UVB-irradiated and PEP005 gel treated mice was similar in thickness to that observed in unirradiated naïve mice. See FIG. 17.

An apparent straightening of dermal collagen fibers following 0.05% PEP005 gel treatment was also evident by H&E staining (FIGS. 11C and D) when compared with untreated mice. See FIGS. 16A and B.

FIG. 16 shows morphological changes induced by UVB-damage and their reversal after PEP005 field treatment.

FIG. 17 illustrates changes in epidermal thickness following 0.05% PEP005 gel field treatment. Epidermal thickness within the treatment area was measured from skin sections from UVB irradiated untreated mice (Control) (n=3), UVB irradiated mice and 0.05% PEP005 gel treated mice (n=3), and unirradiated, untreated, naïve mice (n=2). Control and PEP005 gel treated mice were culled at 21 weeks post PEP005 gel treatment. 9-13 skin thickness measurements were made per mouse (with multiple skin sections per mouse analyzed) at different points within the treatment area. Areas where lesions had developed were excluded.

5.11 Spot-Treatment of Established UV-Induced Lesions with 01-0.25% PEP005 Gel

The aim of this study was to determine if topical treatment with 0.1-0.25% PEP005 gel applied after a lesion had developed could cure UVB-induced lesions. Mice were irradiated 3 times per week until a lesion developed that was clearly visible. Mice with lesions were paired and one lesion from each mouse was treated with either 20 μA of PEP005 gel or placebo gel. The larger lesion within a pair was assigned to the PEP005 group. Four treatment regimens were tested and are summarized in Table 1. Group 1 consisted of mice bearing large (>15 mm²) tumors at time of treatment. These mice were treated with 20 μA of 0.1% PEP005 gel, daily for 2 days. Group 2 consisted of mice bearing smaller tumors (2.3-11.4 mm2) at time of treatment. These mice were also treated with 20 μA of 0.1% PEP005 gel, daily for 2 days. Group 3 consisted of mice bearing small tumors (1.6-8.1 mm2) at the time of treatment. These mice were treated 2 weeks after the last dose of UVB with 20 μA of 0.1% PEP005 gel, daily for 3 days. Group 4 consisted of mice with small tumors (4-9.6 mm²) at time of treatment. These mice were treated 3 weeks after UVB-irradiation had been ceased with 20 μA of 0.25% PEP005 gel, daily for 2 days. Lesions were monitored weekly. Mice were euthanized when the total tumor burden per mouse was considered excessive (usually >100 mm²) A number of mice developed testicular infections and were also euthanized.

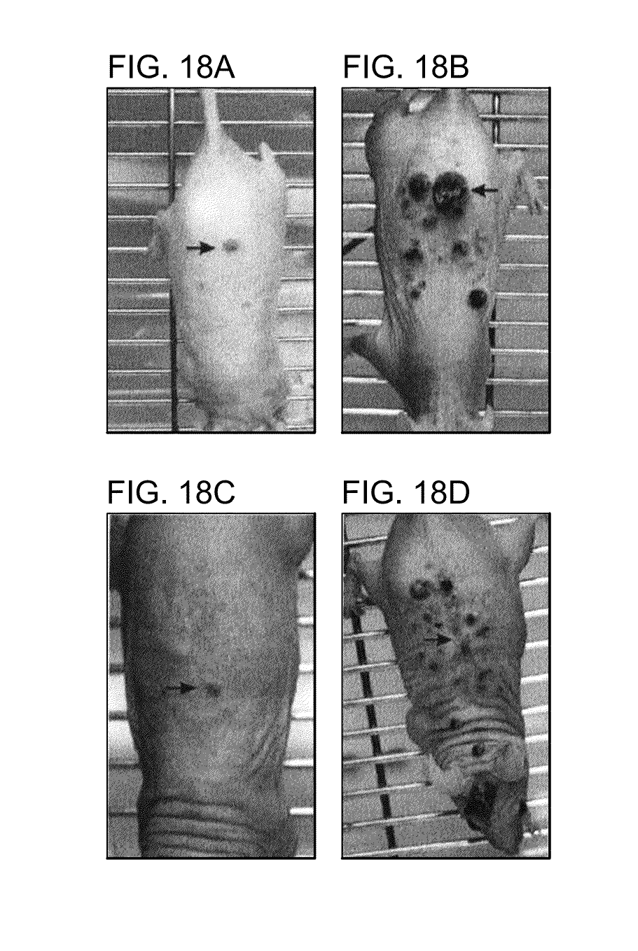

No lesions appeared to be cured by PEP005 gel treatment in any of the treatment regimens (FIG. 18), although it was often difficult to localize the treated tumor in the sea of other tumors that emerged over time (FIG. 18D). Some partial responses did appear to occur (Table 1) albeit with the same caveat. Examples of mice classified as partially cured are shown in FIG. 13. Spot-treatment of smaller lesions with 20 μA of 0.1% PEP005 gel daily for 2 days (total PEP005 dose 40 μg) led to 33% of lesions showing a partial response. This was increased to 57% when a delay of 2 weeks was introduced after UVB irradiation and treatment, and the treatment regimen was increased to 20 μA of 0.1% PEP005 gel daily for 3 days (total PEP005 dose 60 μg). A similar percentage of partial responders (50%) was also achieved when the mice were treated 3 weeks after the last dose of UVB and the dose was escalated to 2 daily applications with 20 μA of 0.25% PEP005 gel (total PEP005 dose 100 μg). However, only two mice were treated in this regimen.

These results suggest that topical PEP005 gel spot-therapy of UVB-induced lesion did not result in any discernable cures in this model. Partial responses appeared to be evident when small lesions (about <13 mm²) were treated, and the response was improved to about 50% when the total PEP005 gel dose was escalated to about >60 μg and there was a 2 weeks delay between the last UVB-irradiation and the first PEP005 gel treatment. The difficulty in identifying individual tumors when other tumors are emerging in and around the treated tumor made it difficult to obtain clear results, illustrating that this is not an ideal model for testing tumor treatment by PEP005 gel.

FIG. 18 depicts topical spot-therapy of UVB-induced lesions treated with PEP005 gel. Mice were irradiated with 1.25 MED of UVB 3 times per week until lesions developed. The mice were treated with 0.1-0.25% PEP005 gel or placebo gel as described in Section 5.11 above. The mice were then monitored for the growth of the treated lesions.

TABLE 1
Summary of treatment of UVB-induced lesions with PEP005 gel

| | | | Time | | | | |
|---|---|---|---|---|---|---|---|
| | | | between | | | | |
| | | | last UVB | | Mean | | |
| | | | and First | | Lesion | Lesion | |
| | | | treatment | | Area | range | Partial |
| Group | Treatment | Regimen | (days) | N/Group | (mm²) | (mm²) | Responders |
| 1 | 0.1%% PEP005 gel | 2 × 20 μl | 1-2 | 7 | 31.4 | 17.2-53.25 | 0 |
| | Placebo gel | | | | 14.5 | 10.85-21.93 | 0 |
| 2 | 0.1%% PEP005 gel | 2 × 20 μl | 1-2 | 6 | 8 | 4.4-11.4 | 2/6 (33%) |
| | Placebo gel | | | | 5.4 | 2.3-9.6 | 0 |
| 3 | 0.1%% PEP005 gel | 3 × 20 μl | 14 | 7 | 6.3 | 2-13 | 4/7 (57%) |
| | Placebo gel | | | | 4.7 | 1.6-8.1 | 0 |
| 4 | 0.025%% PEP005 gel | 2 × 20 μl | 21 | 2 | 7 | 4-9.6 | 1/2 (50%) |
| | Placebo gel | | | | 6 | 4-8 | 0 |

### 6. Conclusions & Recommendations

6.1 PEP005 Field Treatment

Treatment of UVB-damaged skin with 0.05% PEP005 gel resulted in a significant reduction in the number of epidermal UVB-induced p53+ patches (FIG. 7). The p53+ patches represent clones of keratinocytes bearing UV-signature p53 mutations. Keratinocytes bearing UV-induced p53 mutations have selective advantages over normal keratinocytes making them susceptible to accumulating more DNA mutations when exposed to a UV. p53 mutations disrupt cell cycle control and apoptotic pathways. Several lines of evidence suggest that UV-induced mutation of the p53 tumor suppressor gene is an early, event involved in the development of cutaneous SCC. Studies have shown p53 mutations induced by UV radiation are present in 38.5% of photodamaged, chronically sun exposed skin samples (Einspahr et al., 1999), in 53-75% of actinic keratosis (Einspahr et al., 1999; Nelson et al., 1994; Ziegler et al., 1994) and in 54-72% of cutaneous SCC (Brash et al., 1991; Einspahr et al., 1999). Furthermore, heterozygous p53 knockout mice irradiated with UVB developed higher frequencies of p53+ patches and SCC, suggesting that non-functional p53 plays a role in UVB-induced tumor development and progression (Rebel et al., 2001). Thus removal of UVB-induced p53+ patches by topical treatment with 0.05% PEP005 gel suggests a prophylactic utility for PEP005 gel for removal of photodamaged skin and preventing skin cancer development.

Histological analysis of UVB damaged skin treated with 0.05% PEP005 gel showed loss

of the keratinocyte layer and cutaneous mast cells (FIGS. 8A-C), the presence of a neutrophil infiltrate, localized edema with hemorrhaging, followed by eschar formation that resolved within 4-5 weeks after treatment. See FIG. 7A. Twenty one weeks after treatment the epithelial thickness was similar to unirradiated skin and significantly less than that seen in UVB damaged skin. See FIG. 17. Increased numbers of mast cells are associated with chronic exposure of skin to UV both in SKH1 mice and in humans. Their immunosuppressive activities are thought to contribute to the development of UV-induced lesions such as AK and SCC (Ch'ng et al., 2006; Hart et al., 2000; Hart et al., 2001). Field treatment with 0.05% PEP005 gel appeared not only to remove p53+ keratinocytes, but also appeared to reverse the UVB-induced epidermal thickening and mast cell accumulation.

Field treatment with 0.05% PEP005 gel resulted in the treated skin shrinking, with about a 3 fold reduction in area (FIG. 9B and FIG. 11B). This appeared to be due to partial hemoablation of the treated skin, with surrounding skin being pulled towards the treatment area (FIG. 15). Field treatment with 0.05% PEP005 gel resulted in a clear reduction in the number of emerging lesions only when an about 10 cm² area was treated. See FIG. 11. When an about 4 cm² area was treated the reduction in the number of emerging lesion was (i) much less apparent when all the lesions on the mice were considered (FIG. 9A) and (ii) was absent when only lesions within the treatment areas were considered (FIG. 9C). Three potential and not mutually exclusive explanations may explain these observations:

(1) Treatment of the about 10 cm² area represented treatment of the majority of the UVB damaged skin and may also have removed most of the immunosuppressive cells. The behavior of these cells is poorly understood, but they may be able to migrate and/or set up a locally dispersed immunosuppressive environments that promote lesion development (Gabrilovich and Nagaraj, 2009; Stumpfova et al., 2010). Removal of say 20-40% of these cells by treating an about 4 cm² area may conceivably be insufficient to affect significantly the immunosuppressive environment, especially given that the area after treatment was reduced by about 3 fold and most of the skin neighboring the treatment area was also UVB damaged. When considering the about 10 cm² area, much of the skin neighboring the treatment area had received less UVB. In this scenario the removal of the immunosuppressive cells may be equally as important for PEP005 gel field treatment as the removal of p53+ lesions;

(2) The small area that remains after treating an about 4 cm² area may mean that a clear delineation between lesions arising from inside and outside the tattooed area may be difficult, with p53+ cells originating outside the treatment area potentially growing or migrating into the treatment area during the wound healing process after 0.05% PEP005 gel treatment. During wound healing keratinocytes have been shown to migrate (Nataraj an et al., 2006); and

(3) We have assumed that the reduction of p53 patches+ within the treatment area per cm² was similar when about 4 cm² or about 10 cm² area was treated. This would appear likely, but was not demonstrated. Mounting evidence in the literature supports the notion that cutaneous tumors arise from stem cells within skin (Ambler and Maatta, 2009; Gerdes and Yuspa, 2005).

Two main groups of skin stem cells have been described in the literature: (a) stem cells interspersed throughout the basal layer, located in the interfollicular epidermis (IFE). These stem cells are located at the centre of so-called epidermal proliferation units (EPU); and (b) stem cells located within the hair follicle in a well-protected area called the “bulge”. Both populations of stem cells have been implicated as the cells that give rise to cutaneous lesions after UVB irradiation (de Gruijl and Rebel, 2008; Gerdes and Yuspa, 2005), although those arising from the hair follicle bulge appear to have an increased malignant potential (Morris et al., 2000). Recently, evidence has supported the notion that interfollicular p53+ patches arise from p53+ stem cells located in the EPU (de Gruijl and Rebel, 2008). The reduction in the number of p53+ patches following 0.05% PEP005 gel treatment may thus reflect the reduction of mutated EPU stem cells. In contrast, the UVB-damaged stem cells located within the hair follicle bulge may be less accessible to 0.05% PEP005 gel field treatment and may be less readily detected by the staining technique used herein. Conceivably, treatment of the about 10 cm² area may remove more of the mutated cells, and in particular may remove more of the mutated cells in the hair follicle bulge.

This study suggests that prophylactic PEP005 field treatment is only effective in preventing skin cancer development when a sizable area (about 10 cm²) is treated but is less effective when a smaller area is treated (about 4 cm²). This may mean that either treating a relatively large area is important for prophylactic efficacy or that treating a large percentage of photodamaged skin in any particular area is important.

There was no difference in growth or size distribution of lesions after PEP005 gel or placebo gel treatment indicating that tumors emerging after 0.05% PEP005 gel treatment do not behave more aggressively.

6.2 PEP005 for Tattoo Removal

Treatment with 0.05% PEP005 gel appeared to lead to the removal of some of the tattooed lines suggesting that PEP005 gel may find utility in tattoo removal.

6.3 Spot Therapy of UVB-Induced Lesion

Spot-therapy of UVB-induced lesions within a severely photodamaged area with high-dose PEP005 gel (0.1-0.25%) did not lead to discernable cures of any of the lesions treated. However, partial responses following treatment with PEP005 gel did appear to occur. See Table 1. A major problem with this model is the difficulty in determining the effect of treatment of a single lesion when multiple lesions emerge within the same and/or surrounding areas. This model would thus appear largely unsuitable for evaluating the efficacy of topical PEP005 gel for tumor therapy.

### Example 3

Outbred SKH1/hr mice were irradiated 3 times per week (Monday, Wednesday, and Friday) for 10-11 weeks under 6×TL-12/40 W fluorescent tubes (Phillips) mounted in parallel. During the irradiation mice were segregated into individual boxes (11.5×20 cm) with a piece of 0.125 mm cellulose acetate placed over the box to prevent any UVC reaching the mice. The mice were 26 cm below the UV lamps. Under these conditions the mice received 1.25 times the minimal erythemal dose (MED) of UVB at each exposure, with the total UVB dose being 37.5 MED. One MED was defined as the minimal UVB dose, which caused erythema-oedema evident by visual examination. This MED dose had been previously established as suitable for these studies. UVB-irradiation was ceased for 2-7 days if any of the mice within a cohort showed signs of overt erythema, and was resumed once the overt erythema had resolved. Such cessation was usually only necessary after the initial 2-3 doses of UVB, as mice become resistant to UVB-induced erythema with repeated exposures.

1. Immunohistochemical Examination of p53+ Patches in UVB-Damaged Skin Following PEP005 Gel Treatment

One to three days after discontinuation of UVB irradiation, a about 4 cm² dorsal area was treated topically daily for 2 days with 40 μl of PEP005 gel or placebo gel. The square treatment area was demarcated by a tattoo. An untreated group served as an additional control. Two doses of PEP005 gel were tested: 0.01% and 0.05% (w/v) gel. Once the treated area had healed (two weeks for the 0.01% PEP005 gel treatment and 4-5 weeks for the 0.05% PEP005 gel treatment), the mice were sacrificed. The treated areas were excised and separation of the epidermis from the dermis was achieved by floating dorsal skin dermis side down overnight at 4° C. in thermolysin (200 μg/ml, Sigma P1512) supplemented with CaCl2 (1 mM). The epidermis was fixed in 4% formalin for 10 min at room temperature, and was washed in phosphate buffered saline solution (PBS) before being placed in 70% v/v ethanol and stored at 4° C. Antigen retrieval was performed by placing the epidermal samples in boiling 10 mM citrate buffer (pH 6.0) for 5 min. Endogenous peroxidase activity was inhibited by incubating in 1.5% H₂O₂ in methanol for 20 min. The epidermal samples were blocked for 1 h in a solution containing 10% normal rabbit serum (NRbS), 0.2% BSA (Faction V, Sigma, Cat No. A9647) and 0.1% saponin (Sigma, Cat No. 47036) in PBS. The epidermal samples were incubated with a mutant-specific p53 antibody (PAB240, Neomerkers, Cat No. MS-104-P) diluted 1:25 in 10% NRbS/0.2% BSA/0.1% saponin/PBS overnight at 4° C. Unbound primary antibody was removed by washing samples in PBS/0.5% Tween20. Signal amplification and recognition of the primary antibody was performed by incubating the samples with a biotinylated rabbit anti-mouse IgG1 secondary antibody (RbαM (IgG1)-Biotin, Zymed, Cat No. 61-0140) diluted 1:50 in PBS/0.2% BSA/0.1% saponin. The samples were washed in PBS to remove any unbound secondary antibody and incubated for 45 min at room temperature with the Streptavidin/HRP complex (DAKO, Cat No. P0397) diluted 1:200 in PBS. Following 3 washes in PBS, the signal was visualised with DAB (Sigma Cat No. D5905) The epidermal sheets were then mounted basal side up onto glass slides in Keizer's Glycerine (Merk, Cat No. 1.09242.100). A grid placed on top of each epidermal sheet preparation was used to score the number of p53+ patches in each square using a light microscope with a 10 or 20× objective. A patch was defined as an area of closley associated p53 staining nuclei ≧6 in number.

1a. Other Histological Studies on UVB-Damaged Skin Following PEP005 Gel Treatment.

Three days after the last dose of UVB irradiation, a ˜4 cm2 dorsal area was topically treated daily for 2 days with 40 μl of 0.05% PEP005 gel or placebo gel. The treatment area was demarcated by a tattoo. Mice were culled 6 h (6 h) and 24 h (Day 1) after the first dose of 0.05% PEP005 gel and 24 h after the second dose of 0.05% PEP005 gel or placebo gel (Day 2). The samples were fixed in 10% formalin and processed by QIMR histology for paraffin embedding. Paraffin sections were stained with haematoxylin and eosin (H&E), Leder stain or toludine blue by QIMR Histotechnology, in accordance with standard protocols.

1b. Development of UVB-Induced Lesions Following 0.05% PEP005 Gel Treatment.

One to three days after discontinuation of UVB irradiation, dorsal areas previously demarcated by a tattoo were treated topically daily for 2 days with 0.05% PEP005 gel or placebo gel. An untreated group served as an additional control. Treatment of two different skin areas was examined, (i) a ˜4 cm2 area located centrally on the dorsa where the majority of the UVB-induced damaged skin is known to occur, and (ii) a ˜10 cm2 rectangular area located centrally on the dorsa, encompassing the majority of the UVB-damaged skin. Treatment volumes of PEP005 and placebo gels were adjusted according to the treatment area as follows: 40 μl of gel was applied to the ˜4 cm2 square and 100 μl of gel was applied to the ˜10 cm2 reactangle. The mice were monitored weekly for the development of lesions both within and outside the treatment area. Lesions arising on the tattooed lines were considered outside the treatment area. The number and size of skin lesions was monitored over time. At the termination of the study (or earlier in cases where total lesion area was excessive or other welfare issues required euthansia), the mice were sacrificed and fixed whole in 4-10% paraformaldehyde. Treatment areas from control, placebo gel and PEP005 gel treated mice were excised and paraffin embedded. Standard histolgy staining was performed by QIMR Histotechnology.

2. Therapeutic Treatment of U-V Damaged Skin with 0.01-0.05% PEP005 Gel

### 2.a p53 Mutations

Chronic exposure of SKH1/hr mice to UV leads to the development of “UV-signature” p53 mutations at hotspot codons (Benjamin et al., 2008; de Gruijl and Rebel, 2008). Clonal expansion of keratinocytes with p53 mutations leads to the formation of p53 mutant (p53+) patches/foci, which can be detected by immunohistochemistry (IHC) using an antibody that recognises the conformation of mutated p53 (Benjamin et al., 2008; de Gruijl and Rebel, 2008). Although some p53+ keratinocytes spontaneously regress in SKH1/hr mice (as they do in humans), others develop into pre-cancerous lesions resembling AKs and can progress to become SCCs (Rebel et al., 2001). The histology suggests that the reduction in p53+ patches mediated by 0.05% PEP005 gel treatment is a result of direct chemoablation of keratinocytes. It also suggests that 0.05% PEP005 gel treatment results in (i) a neutrophilic infiltrate consistent with previous reports (Ogbourne et al., 2004), and (ii) a loss of cutaneous mast cells. Mast cells have been reported to contribute to a local immunosuppressive environment that supports the development of UVB-induced lesions (Ch'ng et al., 2006; Hart et al., 2000).

### 2.b UV Regimen

Same as section 1 except that UVB-irradiation protocol was continued until visible p53+ patches had formed.

The aim of the study was to determine whether topical field treatment of UV-damaged skin in SKH1/hr mice with 0.05% PEP005 gel led to a reduction in the number of p53+ patches. Mice were irradiated with UV as described in Section 4.3.1. A ˜4 cm2 area on the mice dorsa located at the centre of the UV-damaged skin and demarcated by tattoo was treated daily for 2 days with 40 μl of 0.05% PEP005 gel or placebo gel. A control group remained untreated. Once the treated area had healed (4-5 weeks after treatment), the mice were sacrificed and the epidermis within the treatment area was analysed for the presence of p53+ patches as described in section 1.

Escalating the treatment dose to 0.05% PEP005 gel caused more of a reaction on the skin (FIG. 2A). Treatment with a single dose of 0.05% PEP005 gel caused reddening of the skin, visible within 1 hour of treatment, with erythema increasing with time, and haemorrhaging was evident after 24 h. By 24 h after the second dose, an eschar encompassing the whole treatment area had formed which contracted over time and resolved after 4-5 weeks. After this healing process was complete the mean area within the tattoo had reduced by about 3-fold (FIG. 2B).

Treatment with 0.05% PEP005 gel resulted in a significant reduction (p=0.002) in the number of p53+ patches/cm2 formed within the treatment area. It was harder to separate the epidermis from the dermis in the 0.05% PEP005 gel treatment group and small areas of skin were sometimes lost. However, this was not significant enough to affect the analysis. These data show that topical treatment with 0.05% PEP005 gel removed p53+ patches in this model.

### Example 4

This Example 4 is directed to the prophylactic activity of PEP005 in the field treatment of ultraviolet B radiation (UVB)-damaged skin, and the therapeutic activity of PEP005 in the treatment of UVB-induced skin lesions. The outbred SKH1/hr mouse model of UVB-induced p53 mutant (p53+) patches and lesions (previously established at QIMR) is used to address these aims. Topical field treatment with 0.05% PEP005 of SKH1 mice with photodamaged skin results in significantly fewer p53+ patches/cm², reduction of the UV induced epidermal thickening, and a reduction in cutaneous mast cells. Field treatment of a about 10 cm² dorsal area of skin, which contained most of the UV damaged skin, with 0.05% PEP005 gel results in an about 70% reduction in the number of UVB-induced skin lesions that emerged after UVB irradiation over time. These studies support the utility of PEP005 gel for prophylactic field treatment of UVB damaged skin. Topical spot therapy of established UVB-induced lesions with PEP005 gel (0.1-0.25%) results in no discernable cures.

Thus, this Example 4 is directed to determining the prophylactic and therapeutic potential of PEP005 for treating photodamaged skin and UVB-induced lesions.

For the prophylactic study the following are addressed:

1. To determine whether topical field therapy with 0.01% or 0.05% PEP005 gel would lead to the reduction in the number of UVB-induced p53+ patches.

2. To determine whether field treatment with PEP005 gel would result in a reduction in the number of UVB-induced lesions.

3. To examine the histological changes induced by field treatment with PEP005 gel and determine whether neutrophils are recruited to the treatment area.

For the therapeutic study, it is directed to determining whether topical treatment with high dose PEP005 gel (0.1-0.25%) could cure UVB-induced lesions in the SKH1/hr model.

### 1. Materials and Methods:

1.1 SKH1/hr Model

The SKH1/hr mouse is selected as the model system because: (i) the model generates measurable UV-induced p53 mutations, (ii) some mutant p53 cells are known to progress to AK-like lesions and develop into SCCs, and (iii) this mouse strain is immunocompetent. A breeding program for outbred SKH1 mice has been established at QIMR. Male SKH1 mice are used for this study. To prevent any fighting (which promotes tumours), mice are kept two per cage, with mice separated by a physical barrier. Routine animal husbandry is undertaken by the QIMR Bancroft Centre Animal Facility according their standard operating procedures (SOPs). Feed and water is available ad libitum from individual baskets and water bottles attached to the cages.

1.2 PEP005

PEP005 and placebo gel used in this study were provided by Peplin Inc.
0.25% PEP005 gel
0.1% PEP005 gel
0.05% PEP005 gel
   Once this stock had finished, subsequent 0.05% PEP005 gel was made by mixing 1:1 volumes of PEP005 (0.1% B/N 033C) with placebo gel
0.01% PEP005 gel

1.3 Prophylactic Studies

1.3.1 UVB Regimen

Outbred SKH1/hr mice are irradiated 3 times per week (Monday, Wednesday, and Friday) for 10-11 weeks under 6×TL-12/40 W fluorescent tubes (Phillips) mounted in parallel. During the irradiation mice are segregated into individual boxes (11.5×20 cm) with a piece of 0.125 mm cellulose acetate placed over the box to prevent any UVC reaching the mice. The mice are 26 cm below the UV lamps. Under these conditions the mice received 1.25 times the minimal erythemal dose (MED) of UVB at each exposure, with the total UVB dose being 37.5 MED. One MED is defined as the minimal UVB dose, which caused erythema-oedema evident by visual examination. This MED dose is previously established as suitable for these studies and reported in Cozzi & Suhrbier (2010). UVB-irradiation is ceased for 2-7 days if any of the mice within a cohort showed signs of overt erythema, and is resumed once the overt erythema had resolved. Such cessation is usually only necessary after the initial 2-3 doses of UVB, as mice become resistant to UVB-induced erythema with repeated exposures.

1.3.2 Immunohistochemical Examination of p53+ Patches in UVB-Damaged Skin Following PEP005 Gel Treatment

One to three days after discontinuation of UVB irradiation, a ˜4 cm² dorsal area is treated topically daily for 2 days with 40 μl of PEP005 gel or placebo gel. The square treatment area was demarcated by a tattoo. An untreated group served as an additional control. Two doses of PEP005 gel are tested: 0.01% and 0.05% (w/v) gel. Once the treated area heals (two weeks for the 0.01% PEP005 gel treatment and 4-5 weeks for the 0.05% PEP005 gel treatment), the mice are sacrificed. The treated areas are excised and separation of the epidermis from the dermis is achieved by floating dorsal skin dermis side down overnight at 4° C. in thermolysin (200 μg/ml, Sigma P1512) supplemented with CaCl2 (1 mM). The epidermis is fixed in 4% formalin for 10 min at room temperature, and is washed in phosphate buffered saline solution (PBS) before being placed in 70% v/v ethanol and stored at 4° C. Antigen retrieval is performed by placing the epidermal samples in boiling 10 mM citrate buffer (pH 6.0) for 5 min Endogenous peroxidase activity is inhibited by incubating in 1.5% H₂O₂ in methanol for 20 min. The epidermal samples were blocked for 1 h in a solution containing 10% normal rabbit serum (NRbS), 0.2% BSA (Faction V, Sigma, Cat No. A9647) and 0.1% saponin (Sigma, Cat No. 47036) in PBS. The epidermal samples are incubated with a mutant-specific p53 antibody (PAB240, Neomerkers, Cat No. MS-104-P) diluted 1:25 in 10% NRbS/0.2% BSA/0.1% saponin/PBS overnight at 4° C. Unbound primary antibody is removed by washing samples in PBS/0.5% Tween20. Signal amplification and recognition of the primary antibody was performed by incubating the samples with a biotinylated rabbit anti-mouse IgG1 secondary antibody (RbαM (IgG1)-Biotin, Zymed, Cat No. 61-0140) diluted 1:50 in PBS/0.2% BSA/0.1% saponin. The samples are washed in PBS to remove any unbound secondary antibody and incubated for 45 min at room temperature with the Streptavidin/HRP complex (DAKO, Cat No. P0397) diluted 1:200 in PBS. Following 3 washes in PBS, the signal is visualised with DAB (Sigma Cat No. D5905) The epidermal sheets are then mounted basal side up onto glass slides in Keizer's Glycerine (Merk, Cat No. 1.09242.100). A grid placed on top of each epidermal sheet preparation is used to score the number of p53+ patches in each square using a light microscope with a 10 or 20× objective. A patch is defined as an area of closley associated p53 staining nuclei ≧6 in number.

1.3.3 Other Histological Studies on UVB-Damaged Skin Following PEP005 Gel Treatment.

Three days after the last dose of UVB irradiation, a ˜4 cm² dorsal area is topically treated daily for 2 days with 40 μl of 0.05% PEP005 gel or placebo gel. The treatment area is demarcated by a tattoo. Mice are culled 6 h (6 h) and 24 h (Day 1) after the first dose of 0.05% PEP005 gel and 24 h after the second dose of 0.05% PEP005 gel or placebo gel (Day 2). The samples are fixed in 10% formalin and processed by QIMR histology for paraffin embedding. Paraffin sections are stained with haematoxylin and eosin (H&E), Leder stain or toludine blue by QIMR Histotechnology, in accordance with standard protocols.

1.3.4 Development of UVB-Induced Lesions Following 0.05% PEP005 Gel Treatment.

One to three days after discontinuation of UVB irradiation, dorsal areas previously demarcated by a tattoo are treated topically daily for 2 days with 0.05% PEP005 gel or placebo gel. An untreated group served as an additional control. Treatment of two different skin areas is examined, (i) a ˜4 cm² area located centrally on the dorsa where the majority of the UVB-induced damaged skin is known to occur, and (ii) a ˜10 cm² rectangular area located centrally on the dorsa, encompassing the majority of the UVB-damaged skin. Treatment volumes of PEP005 and placebo gels are adjusted according to the treatment area as follows: 40 μl of gel is applied to the ˜4 cm² square and 100 μl of gel is applied to the ˜10 cm² reactangle. The mice were monitored weekly for the development of lesions both within and outside the treatment area. Lesions arising on the tattooed lines are considered outside the treatment area. The number and size of lesions is monitored over time. At the termination of the study (or earlier in cases where total lesion area is excessive or other welfare issues required euthansia), the mice are sacrificed and fixed whole in 4-10% paraformaldehyde. Treatment areas from control, placebo gel and PEP005 gel treated mice are excised and paraffin embedded. Standard histolgy staining is performed by QIMR Histotechnology.

1.4 Therapeutic Treatment of UVB-Induced Lesions with 0.01-0.25% PEP005 Gel

1.4.1 UVB Regimen

1Same as 1.3.1 except that UVB-irradiation protocol was continued until visible lesions had formed.

1.4.2 Treatment of UVB-Induced Lesions

Once two or more mice develop lesions visible by visual examination, the mice are paired and a lesion from each mouse is treated with either 0.1-0.25% (w/v) PEP005 gel or placebo gel. The mice are monitored over time for growth of the treated lesions. Mice are euthanased when the total lesion area is deemed excessive on ethical grounds (usually >100 mm2) or the mice show clear signs of ill health. Mice are also euthanased for animal welfare issues other than tumour burden where necessary.

1.5 Data Analysis

Statistical analysis was performed using Graphpad Prism version 5.03 (Graphpad Software, San Diego Calif. USA, www.graphpad.com) or SPSS for Windows (version 15.0, 2007; SPSS, Chicago, Ill.).

### 2. Results

2.1 Field Treatment of UV-Damaged Skin with 0.01% PEP005 Gel Did Not Reduce the Number of p53+ Patches

This Example 4 is to determine whether topical field treatment of UVB-damaged skin in SKH1/hr mice with 0.01% PEP005 gel leads to a reduction in the number of p53+ patches. Mice are irradiated with UVB as described in Section 4.3.1. ˜4 cm² areas located at the centre of the UVBdamaged skin on the mice dorsa and demarcated by tattoos are treated daily for 2 days with 40 μl of 0.01% PEP005 gel or placebo gel. A control group remains untreated. Two weeks after treatment the mice are sacrificed and the epidermis within the treatment area is analysed for the presence of p53+ patches as described in Section 1.3.2.

Treatment with 0.01% PEP005 gel induces slight erythema, with flaking and occasionally mild scabbing, which resolves within 2 weeks after treatment, leaving no evidence of scarring or apparent change in the treatment area (FIG. 6 A). Enzymatic separation of the epidermis from the dermis with thermolysin and immunohistochemical analysis of the epidermal sheet show that treatment with 0.01% PEP005 has no significant effect on the number of p53+ patches/cm² compared to placebo-treated or control mice (FIG. 7 B). This data suggests that topical treatment with 0.01% PEP005 has no beneficial effect on UVB-damaged skin in the SKH1/hr mouse model of photodamage. The treatment dose is subsequently increased to 0.05% PEP005 gel (see 2.2).

FIGS. 6 A and B show UV damaged skin that is treated with 0.01% PEP005 gel. SKH1 mice are exposed to 1.25 MED three times per week, for thirty doses. One day after the last UVB irradiation dose, on Day 0 and Day 1, 40 μl of 0.01% PEP005 gel (n=5) or placebo (n=6) gel is applied topically in a ˜4 cm2 area demarcated by tattoo on the dorsa. Control mice (n=5) remains untreated. On Day 13 (2 weeks after UVB irradiation had ceased) the mice are euthanased and the epidermis within the tattooed area analyzed for p53+ patches. A: Representative photos before (Day 0) and after (Days 1-13) treatment with placebo and 0.01% PEP005 gels. B: Number of p53+ patches/cm2. Error bars represent the standard error of the mean (SEM).

2.2 Field Treatment of UV-Damaged Skin with 0.05% PEP005 Gel LED to a Reduction in the Number of p53+ Patches

This Example 4 is to determine whether topical field treatment of UVB-damaged skin in SKH1/hr mice with 0.05% PEP005 gel led to a reduction in the number of p53+ patches. Mice are irradiated with UVB as described in Section 4.3.1. A ˜4 cm² area on the mice dorsa located at the centre of the UVB-damaged skin and demarcated by tattoo is treated daily for 2 days with 40 μl of 0.05% PEP005 gel or placebo gel. A control group remained untreated. Once the treated area has healed (4-5 weeks after treatment), the mice are sacrificed and the epidermis within the treatment area is analysed for the presence of p53+ patches as described in Section 1.3.2.

Escalating the treatment dose to 0.05% PEP005 gel caused more of a reaction on the skin (FIG. 7A). Treatment with a single dose of 0.05% PEP005 gel caused reddening of the skin, visible within 1 hour of treatment, with erythema increasing with time, and haemorrhaging was evident after 24 h. By 24 h after the second dose, an eschar encompassing the whole treatment area had formed which contracted over time and resolved after 4-5 weeks. After this healing process was complete the mean area within the tattoo had reduced by ˜3-fold (FIG. 7B).

Treatment with 0.05% PEP005 gel resulted in a significant reduction (p=0.002) in the number of p53+ patches/cm² formed within the treatment area. It was harder to separate the epidermis from the dermis in the 0.05% PEP005 gel treatment group and small areas of skin were sometimes lost. However, this was not significant enough to affect the analysis. These data show that topical treatment with 0.05% PEP005 gel removed p53+ patches in this model.

We observed that in the 0.05% PEP005 gel treatment group the tattoo marking was often removed, possibly at sites where the gel had spread over the tattoo. This suggested that PEP005 gel may find a utility as a tattoo removal agent. We further examined this potential new cosmetic indication for PEP005 gel in a small cohort of mice. A report for this study will be submitted as a separate extension to this contract.

FIGS. 7A and B show UVB damaged skin treated with 0.05% PEP005 gel. SKH1/hr mice were exposed to 1.25 MED three times per week, for thirty doses. One day after the last UVB dose, on Days 0 and 1, 40 μl of 0.05% PEP005 gel or placebo gel was applied topically in the ˜4 cm² areas demarcated by tattoo on the mouse dorsa. Control mice remained untreated. Once the treatment area had healed (4-5 weeks after treatment) the mice were euthanased and the treated epidermis analysed for the presence of p53+ patches by IHC. A: representative photographs before, during and after treatment with 0.05% PEP005 gel. B: The number of p53+ patches/cm² within the treatment areas 4-5 weeks after treatment. Control (n=20), placebo gel (n=17), PEP005 gel treatment (n=22). Error bars represent the SEM. ** (p=0.002, Placebo vs PEP005, Mann Whitney U test).

2.3 Histological Changes Induced by Treating UVB-Damaged Skin with 0.05% PEP005 Gel

To identify the histological changes after 0.05% PEP005 gel treatment, skin is examined at different times after treatment; 6 h after the first dose is applied (6 h), 24 h after the first dose is applied (Day 1), and 24 h after the second dose is applied (Day 2). Placebo treated mice are treated with equivalent volumes of placebo gel and the skin examined 24 h after the second dose (FIG. 8).

Placebo-treated skin (FIG. 8A) show the expected epidermal thickening that arises after UVB-irradiation (Chaquour et al., 1995). Six hours after the first treatment with 0.05% PEP005 gel, H&E staining show a reduction in haematoxylin staining in the epidermal keratinocytes (FIG. 8A, 6 h), consistent with necrosis of keratinocytes. Twenty four hours after treatment with 0.05% PEP005 gel (Day 1), the epidermal layer is no longer clearly visible (FIG. 8A, Day 1). By Day 2 (24 h after the second dose of 0.05% PEP005 gel), extensive hemorrhaging is evident within the treatment area (FIG. 8A).

To assess whether neutrophils are recruited to the site after treatment with 0.05% PEP005 gel, sections were stained with Leder stain that detects the enzyme chloroacetate esterase present in granulocytes. Leder stain stains granulocytes red-pink (FIG. 8B). Concomitant with the loss of epidermal keratinocytes and epidermal/dermal mast cells and the induction of local haemorrhaging, 0.05% PEP005 gel treatment also led to the recruitment of neutrophils to the treatment site. This was apparent 6 h after the first treatment (FIG. 8B, 6 h) and was extensive 24 h after the second treatment (FIG. 8B, Day 2). The neutrophils stained positively for chloroacetate esterase (FIG. 8B, PEP005) with multi-lobed nuclei apparent when counter stained with haematoxylin. The cells did not stain pink-purple with toluidine blue (FIG. 8C, PEP005) illustrating they were not mast cells.

To determine whether mast cells were present, parallel sections were stained with toluidine blue, which stains mast cells pink-purple. In agreement with the literature (Ch'ng et al., 2006; Brand et al., 2002), UVB-irradiated skin contained abundant mast cells (FIG. 8C, Placebo). Treatment with 0.05% PEP005 gel led to a reduction in the intensity of toluidine blue staining of mast cells by 6 h, indicative of mast cell degranulation and/or death (FIG. 8C, 6 h). By 24 h, there were very few mast cells in the skin (FIG. 8C, Day 1) and this is also true 24 h after the second dose of 0.05% PEP005 gel (FIG. 8C, Day 2).

The histology suggests that the reduction in p53+ patches mediated by 0.05% PEP005 gel treatment is a result of direct chemoablation of keratinocytes. It also suggests that 0.05% PEP005 gel treatment results in (i) a neutrophilic infiltrate consistent with previous reports (Ogbourne et al., 2004), and (ii) a loss of cutaneous mast cells. Mast cells have been reported to contribute to a local immunosuppressive environment that supports the development of UVB-induced lesions (Ch'ng et al., 2006; Hart et al., 2000).

FIG. 8 shows histology of UV damaged skin hat is treated with placebo or 0.05% PEP005 gel. SKH1 mice were exposed to 1.25 MED three times per week, for thirty doses. Three days after cessation of irradiation, on Day 0 and Day 1, 40 μl of 0.05% PEP005 gel or placebo gel was applied topically to a ˜4 cm² areas demarcated by tattoos on the mouse dorsa. Pictures were taken 6 h (Day 0-6 h) and 24 h after first dose of 0.05% PEP005 gel (Day 1, PEP005) and 24 h after the second 0.05% PE005 gel treatment (Day 2, PEP005). Placebo samples were taken 24 h after the second dose with placebo gel (Day 2, Placebo). A: H&E. staining of skin sections. B: Leder stain (red-pink colour) of chloracetate esterase found in granulocytes (which includes neutrophils and mast cells). Counter staining with haematoxylin (blue) showing cell nuclei. C: Toluidine blue staining. Mast cells stain pink-purple. Arrows point at examples of mast cells. Insets in C show mast cells at higher magnification. Bar represents ˜200 μm

2.4. Field Treatment of a ˜4 cm² Area of UVB-Damaged Skin with 0.05% PEP005 Gel Did not Affect the Formation of UVB-Induced Epidermal Lesions

The results in Section 2.1 suggest that treatment with 0.05% PEP005 gel leads to the reduction in photodamaged keratinocytes, with significantly fewer p53+ patches present after treatment. To determine whether the reduction of UVB-damaged keratinocytes would correlate with reduced formation of UVB-induced lesions, the same experimental design is used as described in Section 2.2. Briefly, SKH1/hr mice are irradiated with 30 doses of 1.25 MED of UVB over 10-11 weeks. The mice are then randomised into three groups and ˜4 cm² areas demarcated by a tattoo on the mice dorsa are treated daily for 2 days with 40 μl of 0.05% PEP005 gel or placebo gel. An untreated control group was also included. Following treatment the mice are monitored weekly for the development of lesions both within and outside the treatment area. Both the number and size of the lesions is recorded.

Treatment with 0.05% PEP005 gel leads to a small decrease in the total number of UVB-induced lesions per mouse that emerged over time (FIG. 9A) Significance between placebo gel and PEP005 gel treated mice is reached at 13, 15, and 20 weeks post-treatment (FIG. 9A, *). Control mice also appear to show a reduced number of lesions when it is compared to placebo treated mice (FIG. 9A). This may be a consequence of placebo gel treated mice scratching after application of the gel. Scratching is known to increase the number of lesions.

Twenty weeks after treatment with 0.05% PEP005 gel, the area within the tattoo had contracted by ˜3-fold compared to placebo gel and control mice (FIG. 9B). Although there was a small reduction in the number of lesions per mouse after 0.05% PEP005 gel treatment (FIG. 9A), there was no significant reduction in the number of lesions per cm² within the treatment area (FIG. 9C). The reduction in the number of p53+ lesions within the treatment area thus did not appear to correlate with the reduction in the number of UVB induced lesions.

FIG. 9 shows the development of UV-induced lesions after no treatment, placebo treatment or field treatment with 0.05% PEP005 gel in a ˜4 cm² area. SKH1/hr mice are exposed to 1.25 MED three times per week, for thirty doses. One to 3 days after the last irradiation, the mice are treated topically, daily for 2 days (week 0) with 40 μl of 0.05% PEP005 gel in a ˜4 cm² area demarcated by a tattoo on the dorsa of the mice. Control mice remain untreated. The mice are then examined weekly for the development of UVB-induced lesions. A: Number of dorsal lesions per mouse. Placebo vs. PEP005, # p<0.05, *p<0.005, Mann Whitney U tests. One mouse in the PEP005 gel treated group is euthanased due to excessive tumour burden and another due to a groin infection. In the placebo gel treated group 2 mice are euthanased due to infected groins. In the Control group 1 mouse is euthanased due to excessive tumour burden. Averages only included data from living animals. B: Reduction in treatment area after 0.05% PEP005 gel treatment. Exclusions as in A plus an additional mouse in the Control group that is euthanased due to groin infection. * p=0.002, Mann Whitney U test. C: Number of dorsal lesions within the tattooed area/cm². Exclusions as for B. There is no significant differences between PEP005 and the other groups at weeks 8 and 9.

2.5 Field Treatment of a ˜10 cm² Area of UVB-Damaged Skin with 0.05% PEP005 Gel Reduced the Number of UVB-Induced Epidermal Lesions

SKH1/hr mice are exposed to 1.25 MED three times per week for a total of 30 doses. One day after the final irradiation dose, the mice are randomised into 3 groups and ˜10 cm² areas demarcated by tattoos on the dorsa were treated topically daily for 2 days with ˜100 μl of 0.05% PEP005 gel or placebo gel. Control mice remained untreated. The mice were examined weekly for the development of UV-induced lesions. The number and size of the lesions located within and outside the treatment area were documented.

In addition to the tattooed treatment area, other lines parallel to the treatment rectangle are also tattooed on the mice to track skin movement following 0.05% PEP005 gel treatment. FIG. 10 shows mice from the control group (A-C), placebo gel treated group (D-F) and PEP005 gel treated group (G-I), 20 weeks after treatment. The photographs show the spectrum of UVB-induced lesions that developed in untreated, placebo and PEP005 treated mice. They also show the favourable cosmetic outcomes and the treatment site contractions (FIGS. 10G&I), which were also seen after treatment of ˜4 cm² areas with PEP005 gel. A slight darker pink residual tainted area remained within the PEP005 treatment area (FIGS. 10G&I), which was not significantly firmer to the touch than normal skin.

FIG. 10 shows photographs of mice 21 weeks post-treatment. Outbred male SKH1/hr mice are exposed to 1.25 MED three times per week, for thirty doses. One day after of the last irradiation dose, ˜10 cm² dorsal areas demarcated by a tattoo are treated topically, daily for 2 days (week 0) with 100 μl of 0.05% PEP005 gel or placebo gel. Control mice remained untreated. A-C: Representative photographs from control mice. D-F: Representative photos of placebo gel-treated mice. H-I: Representative photographs of PEP005gel-treated mice displaying the spectrum of lesions that developed in these mice. G shows a PEP005 gel treated mouse with no lesions. Photographs are taken 21 weeks post-treatment.

Analysis of lesion numbers in the dorsal area show that 0.05% PEP005 gel treatment resulted in a significant ˜70% reduction in the number of dorsal lesions that developed per mouse (FIG. 11A). Treatment with 0.05% PEP005 gel again led to a significant contraction (p<0.001) of the treatment area (FIG. 11B). In contrast with the data shown in FIG. 9C (where the treatment area was ˜4 cm²), field treatment of ˜10 cm² area with PEP005 gel resulted in a significant reduction in the number of lesions per cm² within the treatment area (FIG. 11C). The ability of field treatment with PEP005 gel to prevent development of lesions was thus clearly apparent only when a larger area, that represented the majority of the UVB damaged skin, was treated.

To determine whether treatment of UVB-damaged skin with 0.05% PEP005 gel had an effect on lesion development outside the treatment area, the number of lesions outside the tattooed area was enumerated. Treatment with 0.05% PEP005 gel had no effect on the number of lesions that developed outside the treatment area (FIG. 11D), suggesting that there was no systemic anti-cancer activity associated with PEP005 field treatment in this model.

The results described above illustrated that when the majority of photodamaged skin was treated with 0.05% PEP005 gel, a clearly apparent reduction in the number of skin lesions was observed, with both the total number of lesions per mouse significantly reduced (FIG. 11A) and the number of lesions per cm² within the treatment area significantly reduced (FIG. 11C). To determine whether 0.05% PEP005 gel treatment had an effect on tumour burden, Kaplan-Myer survival curves were generated by analysing the total dorsal lesion area per mouse (FIG. 11E) and the total lesion area within the treatment area over time (FIG. 11F). For both analyses, an event was assigned when the combined lesion area of a mouse reached 70 mm². Both analyses showed that 0.05% PEP005 gel treatment led to a significant reduction in the number of mice that had a cumulative lesion area ≧70 mm2 when compared with placebo gel-treated or control mice. Thus both the number of lesions (FIG. 11A, C) and the tumour burden (as determined by tumour area) (FIG. 11E, F) was significantly reduced by field treatment with 0.05% PEP005 gel.

FIG. 11 shows the development of UV-induced epidermal lesions after no treatment, placebo treatment, or field treatment with 0.05% PEP005 gel in a ˜10 cm² area. SKH1/hr mice were exposed to 1.25 MED three times per week, for thirty doses. One day after the last irradiation, mice were treated topically, daily for 2 days (week 0) with ˜100 μl of 0.05% PEP005 gel or placebo gel over a ˜10 cm² area demarcated by a tattoo on the dorsa. Control mice remained untreated. The mice were examined weekly for the development of UV-induced lesions. A: Total number of dorsal lesions per mouse over time. Five placebo mice were euthanased prior to the end of the study due to excessive tumour burden. Two control mice were euthanased prior to the end of the study due to groin infection and swollen abdomen. A third control mice died for unknown reasons. After death the lesion numbers for these mice were no longer included in the mean. # p<0.05 and * p<0.001, Mann Whitney U test, PEP005 vs. placebo. B: Area of the tattooed square measured 21 weeks after treatment. Exclusions as for A. * denotes p<0.001, Mann Whitney U test. C: Number of dorsal lesions within the tattooed area/cm². Exclusions as for A. # indicates p<0.005 and * indicates p<0.001, Mann Whitney U test, PEP005 vs. placebo. D: Number of dorsal lesions outside the tattooed square. Exclusions as for A. E: Kaplan-Myer curves showing the percentage of mice with a total lesion area of <70 mm². The 3 control mice were excluded as death was unrelated to tumour burden. p=0.002, log rank test, PEP005 vs. placebo. F: Kaplan-Myer curves showing the percentage of mice with a total lesion area within the treatment area of <70 mm². One PEP005 gel treated mouse was excluded as excessive total tumour burden required euthanasia prior to the tumour burden within the treatment area reaching 70 mm². One placebo mouse was excluded for the same reason, and another was excluded as lesions inside and outside the treatment area coalesced. Control mouse exclusion as in A. p<0.001, log rank test, PEP005 vs. placebo.

2.6 Lesions Arising after PEP005 Gel Field Treatment Show No Difference in Growth Kinetics.

To determine whether there is any difference between the growth kinetics of lesions formed between treatment groups, we examined the growth rates of individual lesions that developed within the treatment areas. Week 0 was assigned as the week prior to an individual lesion being identified. Thus all the individual lesions' growth curves start at week 0, irrespective of the time of treatment. The growth curves in FIG. 12 represent the growth of individual lesions from control mice (A), placebo gel treated mice (B), and 0.05% PEP005 gel treated mice (C). The mean lesion growth for each group is shown in FIG. 12D. There was no significant difference in lesion growth between control, placebo and PEP005 gel treated mice. These data show that the tumours that emerged after PEP005 gel treatment did not grow faster than those in control or placebo-treated mice, suggesting that field treatment with PEP005 gel did not resulted in, or select for, the emergence of more aggressive tumours.

FIG. 12 shows growth curves of UVB-induced lesions. Outbred male SKH1/hr mice were exposed to 1.25 MED three times per week, for thirty doses. One day after of the last irradiation dose, the mice were treated topically, daily for 2 days with ˜100 μl of 0.05% PEP005 gel or placebo gel over a ˜10 cm² area demarcated by a tattoo on the dorsa. Control mice remained untreated. The mice were examined weekly for the development of UV-induced lesions. Week 0 was assigned as the week prior to an individual lesion being identified. Thus all the individual lesions' growth curves start at week 0, irrespective of the time of treatment of the mouse. All the lesions shown grew within the tattooed areas. A: Growth curves of individual lesions arising in control mice. B: Growth curves of individual lesions arising in placebo gel treated mice. C: Growth curves of individual lesions arising after 0.05% PEP005 gel treatment. D: Mean growth rates of lesions from A, B and C.

2.7 Lesions Arising after PEP005 Gel Field Treatment Showed Similar Size Distribution to Placebo-Treated and Control Mice

To determine whether the lesions that arose after PEP005 gel treatment showed any changes in size profile, the size distribution of lesions within the treatment area 21 weeks post-treatment is analysed. Bar graphs show the area (mm²) of individual lesions 21 weeks post treatment in control mice (FIG. 8A), placebo gel treated mice (FIG. 13B) and PEP005 gel treated mice (FIG. 13C). Although a significantly higher number of lesions were examined in control and placebo gel treated mice compared to 0.05% PEP005 gel treated mice, a similar size distribution of lesions was present in all three groups. Furthermore, when lesions were grouped into 5 categories based on size ranges, there was a similar distribution profile (FIG. 13D). This data illustrated that in this setting lesions arising after PEP005 gel treatment were similar in size to those arising in control and placebo gel treated mice.

FIG. 13 shows size distributions of lesions at week 21 post-treatment. Outbred male SKH1/hr mice were exposed to 1.25 MED three times per week, for thirty doses.

One day after the last irradiation dose, the mice were treated topically, daily for 2 days with ˜100 μl of 0.05% PEP005gel or placebo gel over a ˜10 cm² area demarcated by a tattoo on the dorsa. Control mice remained untreated. The mice were then examined weekly for the development of UV-induced lesions. A-C: Bar graph of the size of individual lesions 21 weeks post-treatment in Control (A), placebo gel treated (B) and 0.05% PEP005 gel treated (C) mice. Lesions were sorted smallest to largest area. D: Distribution of lesion areas. Tumour lesions from control, placebo gel and PEP005 gel treated mice were grouped into 5 groups according to the specified area ranges. Bars represent the number of lesions within each area range.

2.8 Histological Analysis of UVB-Induced Lesions and Skin Treated with 0.05% PEP005 Gel

H&E staining of representative control (FIG. 14A-D) and placebo gel treated mice (FIG. 14C-D) confirmed that UVB irradiation led to the formation of lesions that are of epidermal origin. The presence of multiple lesions within the one area was evident at lower magnification (FIGS. 14A&C). Higher magnification (FIGS. 14B&D) shows the presence of abundant keratin swirls between the cells. Three mice per group were analysed.

Low magnification images of 0.05% PEP005 gel treated skin from two mice with no visible lesions showed that the skin was also free of histologically observable lesions (FIGS. 14E&G). Most of the skin appeared to have reverted back to resemble unirradiated naïve skin (FIG. 14G) although the occasional area of thickened epidermis was still present (FIGS. 14F&H). Three mice per group are analysed.

FIG. 13 shows H&E staining of skin sections. Outbred male SKH1/hr mice were exposed to 1.25 MED three times per week, for thirty doses. One day after the last irradiation dose, the mice are treated topically, daily for 2 days over a ˜10 cm² area with ˜100 μl of 0.05% PEP005 gel or placebo gel. Skin from untreated control mouse at 21 weeks (A-B). Skin from placebo gel treated mouse analysed 17 weeks after treatment (C & D). Skin from PEP005 gel treated mouse 21 weeks after treatment. Bars represent ˜500 μm.

2.9 Skin Contraction Following Field Treatment with 0.05% PEP005 Gel

In the current study treatment of UVB-damaged skin with 0.05% PEP005 gel resulted in apparent chemoablation of the epithelial skin layer. A neutrophil-mediated response was apparent within 24 h of treatment (FIG. 8), followed by the formation of a scab 24 h after the second dose of PEP005 gel (FIG. 7A). At 20-21 weeks after 0.05% PEP005 gel treatment, skin had contracted, with a reduction in area of ˜3 fold (FIGS. 9B and 9B). To better understand how this skin contraction affected neighbouring skin, lines were tattooed parallel to the tattooed ˜10 cm² treatment rectangles. A schematic diagram is shown in FIG. 15A. When the mice are euthanased (21 weeks after treatment) measurements of the distances between the lines in each group was recorded (FIG. 15B-F). A significant reduction (p<0.002) is only seen in the head to tail and left to right measurements (FIGS. 15E&F). This suggests that the contraction of skin after PEP005 gel treatment resulted in surrounding skin being pulled towards the treatment area.

FIG. 15 shows skin contraction after field treatment with PEP005. Outbred male SKH1/hr mice were exposed to 1.25 MED three times per week, for thirty doses. One day after the last irradiation dose, the mice were treated topically, daily for 2 days with ˜100 μl of 0.05% PEP005 gel or placebo gel over a ˜10 cm² area demarcated by tattoos on the dorsa. Control mice remained untreated. The following mice are excluded from the analysis as they did not complete the study. Five placebo mice were euthanased prior to the end of the study due to excessive tumour burden. Two control mice were euthanased prior to the end of the study due to groin infection and swollen abdomen. A third control mice died for unknown reasons. A: Lines parallel to the treatment rectangle were also tattooed. A schematic diagram of the measurements made after mice were euthanased is shown. B-F: show the different measurements in control, placebo gel and PEP005 gel treated mice. * indicates p<0.002, Placebo vs. PEP005 Mann Whitney U test.

2.10 Morphological Changes Induced by UVB-Damage and their Reversal after Field Treatment with 0.05% PEP005 Gel

Visual examination shows that by 21 weeks after 0.05% PEP005 gel treatment, the skin is healed with a favourable cosmetic outcome, in many cases devoid of UVB-induced lesions (FIG. 10G-I). Histological analyses by H&E staining of dorsal skin from control mice shows that UVB-induced epidermal thickening is maintained 21 weeks after irradiation cessation (FIG. 16A-B). Re-epithelisation following 0.05% PEP005 gel treatments resulted in an epithelial layer with a thickness (FIG. 16C-D) similar to that found in unirradiated naïve mice (FIG. 16E-F). Quantitative analysis of the thickness of the epithelial layer in histological sections of skin from UVB-irradiated control mice, 0.05% PEP005 gel treated mice and non-irradiated mice, showed that the epidermis from UVB-irradiated control mice was thicker than that in UVB-irradiated and 0.05% PEP005 gel treated mice (FIG. 17). The regenerated epidermis in UVB-irradiated and PEP005 gel treated mice was similar in thickness to that observed in unirradiated naïve mice (FIG. 17).

An apparent straightening of dermal collagen fibres following 0.05% PEP005 gel treatment is also evident by H&E staining (FIGS. 16C&D) when compared with untreated mice (FIGS. 16A&B).

FIG. 16 shows morphological changes induced by UVB-damage and their reversal after PEP005 field treatment. A-D: Dorsal skin H&E stained sections from outbred male SKH1/hr mice exposed to 1.25 MED three times per week, for thirty doses. One day after irradiation, the mice were treated topically, daily for 2 days (week 0) with ˜100 μl of 0.05% PEP005 gel over a ˜10 cm² area demarcated by a tattoo on the dorsa. A-B: Skin from control mice fixed in paraformaldehyde. C-D: Twenty one weeks after PEP005 gel treatment mice were sacrificed and skin fixed in paraformaldehyde. E-F: Dorsal skin sections from naïve inbred unirradiated female SKH1/hr mice, fixed in 10% formalin. Bars represent ˜200 μm.

FIG. 17. Changes in epidermal thickness following 0.05% PEP005 gel field treatment. Epidermal thickness within the treatment area was measured from skin sections from UVB irradiated untreated mice (Control) (n=3), UVB irradiated mice and 0.05% PEP005 gel treated mice (n=3), and unirradiated, untreated, naive mice (n=2). Control and PEP005 gel treated mice were culled at 21 weeks post PEP005 gel treatment. 9-13 skin thickness measurements were made per mouse (with multiple skin sections per mouse analysed) at different points within the treatment area. Areas where lesions had developed were excluded.

2.11 Spot-Treatment of Established UV-Induced Lesions with 01-0.25% PEP005 Gel

Another aim of this Example 4 is to determine if topical treatment with 0.1-0.25% PEP005 gel applied after a lesion has developed could cure UVB-induced lesions. Mice are irradiated 3 times per week until a lesion is developed that is clearly visible. Mice with lesions were paired and one lesion from each mouse was treated with either 20 μA of PEP005 gel or placebo gel. The larger lesion within a pair is assigned to the PEP005 group. Four treatment regimens are tested and are summarized in Table 1. Group 1 consisted of mice bearing large (>15 mm²) tumours at time of treatment. These mice are treated with 20 μA of 0.1% PEP005 gel, daily for 2 days. Group 2 consisted of mice bearing smaller tumours (2.3-11.4 mm²) at time of treatment. These mice are also treated with 20 μA of 0.1% PEP005 gel, daily for 2 days. Group 3 consisted of mice bearing small tumours (1.6-8.1 mm2) at the time of treatment. These mice are treated 2 weeks after the last dose of UVB with 20 μA of 0.1% PEP005 gel, daily for 3 days. Group 4 consisted of mice with small tumours (4-9.6 mm²) at time of treatment. These mice are treated 3 weeks after UVB-irradiation has been ceased with 20 μA of 0.25% PEP005 gel, daily for 2 days. Lesions are monitored weekly. Mice are euthanased when the total tumour burden per mouse was considered excessive (usually >100 mm²) A number of mice developed testicular infections and are also euthanased.

No lesions appear to be cured by PEP005 gel treatment in any of the treatment regimens (FIG. 18), although it is often difficult to localize the treated tumour in the sea of other tumours that emerged over time (FIG. 18D). Some partial responses did appear to occur (Table 1) albeit with the same caveate. Examples of mice classified as partially cured are shown in FIG. 13. Spot-treatment of smaller lesions with 20 μl of 0.1% PEP005 gel daily for 2 days (total PEP005 dose 40 μg) led to 33% of lesions showing a partial response. This was increased to 57% when a delay of 2 weeks was introduced after UVB irradiation and treatment, and the treatment regimen was increased to 20 μA of 0.1% PEP005 gel daily for 3 days (total PEP005 dose 60 μg). A similar percentage of partial responders (50%) was also achieved when the mice were treated 3 weeks after the last dose of UVB and the dose was escalated to 2 daily applications with 20 μA of 0.25% PEP005 gel (total PEP005 dose 100 μg). However, only two mice were treated in this regimen.

These results suggest that topical PEP005 gel spot-therapy of UVB-induced lesion did not result in any discernable cures in this model. Partial responses appeared to be evident when small lesions (<13 mm2) were treated, and the response was improved to ˜50% when the total PEP005 gel dose was escalated to >60 μg and there was a 2 weeks delay between the last UVB-irradiation and the first PEP005 gel treatment. The difficulty in identifying individual tumours when other tumours are emerging in and around the treated tumour made it difficult to obtain clear results, illustrating that this is not an ideal model for testing tumour treatment by PEP005 gel.

FIG. 18 shows topical spot-therapy of UVB-induced lesions treated with PEP005 gel. Mice were irradiated with 1.25 MED of UVB 3 times per week until lesions developed. The mice were treated with 0.1-0.25% PEP005 gel or placebo gel as described in Section 2.11. The mice were then monitored for the growth of the treated lesions. A & C: examples of mice pre-treatment. B & D: examples of mice at the end of the experiment. Arrows indicate the position of treated lesions.

TABLE 1
Summary of treatment of UVB-induced lesions with PEP005 gel

| | | | Time | | | | |
|---|---|---|---|---|---|---|---|
| | | | between | | | | |
| | | | last UVB | | Mean | | |
| | | | and first | | Lesion | Lesion | |
| | | | treatment | | Area | range | Partial |
| Group | Treatment | Regimen | (days) | N/Group | (mm²) | (mm²) | Responders |
| 1 | 0.1%% PEP005 gel | 2 × 20 μl | 1-2 | 7 | 31.4 | 17.2-53.25 | 0 |
| | Placebo gel | | | | 14.5 | 10.85-21.93 | 0 |
| 2 | 0.1%% PEP005 gel | 2 × 20 μl | 1-2 | 6 | 8 | 4.4-11.4 | 2/6 (33%) |
| | Placebo gel | | | | 5.4 | 2.3-9.6 | 0 |
| 3 | 0.1%% PEP005 gel | 3 × 20 μl | 14 | 7 | 6.3 | 2-13 | 4/7 (57%) |
| | Placebo gel | | | | 4.7 | 1.6-8.1 | 0 |
| 4 | 0.25%% PEP005 gel | 2 × 20 μl | 21 | 2 | 7 | 4-9.6 | 1/2 (50%) |
| | Placebo gel | | | | 6 | 4-8 | 0 |

### 3. Conclusions & Recommendations

3.1 PEP005 Field Treatment

Treatment of UVB-damaged skin with 0.05% PEP005 gel results in a significant reduction in the number of epidermal UVB-induced p53+ patches (FIG. 2). The p53+ patches represent clones of keratinocytes bearing UV-signature p53 mutations. Keratinocytes bearing UV-induced p53 mutations have selective advantages over normal keratinocytes making them susceptible to accumulating more DNA mutations when exposed to a UV. p53 mutations disrupt cell cycle control and apoptotic pathways. Several lines of evidence suggest that UV-induced mutation of the p53 tumor suppressor gene is an early, event involved in the development of cutaneous SCC. Studies have shown p53 mutations induced by UV radiation are present in 38.5% of photodamaged, chronically sun exposed skin samples (Einspahr et al., 1999), in 53-75% of actinic keratosis (Einspahr et al., 1999; Nelson et al., 1994; Ziegler et al., 1994) and in 54-72% of cutaneous SCC (Brash et al., 1991; Einspahr et al., 1999). Furthermore, heterozygous p53 knockout mice irradiated with UVB developed higher frequencies of p53+ patches and SCC, suggesting that non-functional p53 plays a role in UVB-induced tumour development and progression (Rebel et al., 2001). Thus removal of UVB-induced p53+ patches by topical treatment with 0.05% PEP005 gel provides prophylactic utility for PEP005 gel for removal of photodamaged skin and preventing skin cancer development.

Histological analysis of UVB damaged skin treated with 0.05% PEP005 gel also shows loss of the keratinocyte layer and cutaneous mast cells (FIG. 3), the presence of a neutrophil infiltrate, localised edema with hemorrhaging, followed by eschar formation that resolved within 4-5 weeks after treatment (FIG. 2A). Twenty one weeks after treatment the epithelial thickness was similar to unirradiated skin and significantly less than that seen in UVB damaged skin (FIG. 12). Increased numbers of mast cells are associated with chronic exposure of skin to UV both in SKH1 mice and in humans. Their immunosuppressive activities are thought to contribute to the development of UV-induced lesions such as AK and SCC (Ch'ng et al., 2006; Hart et al., 2000; Hart et al., 2001). Field treatment with 0.05% PEP005 gel appear not only to remove p53+ keratinocytes, but also appeared to reverse the UVB-induced epidermal thickening and mast cell accumulation.

Field treatment with 0.05% PEP005 gel resulted in the treated skin shrinking, with a ˜3 fold reduction in area (FIGS. 4B & 6B). This appeared to be due to partial chemoablation of the treated skin, with surrounding skin being pulled towards the treatment area (FIG. 10).

Field treatment with 0.05% PEP005 gel results in a clear reduction in the number of emerging lesions when a ˜10 cm² area is treated (FIG. 6). When a ˜4 cm² area is treated the reduction in the number of emerging lesion is (i) much less apparent when all the lesions on the mice are considered (FIG. 4A) and (ii) is absent when only lesions within the treatment areas were considered (FIG. 4C). Three potential and not mutually exclusive explanations may explain these observations. (1) Treatment of the ˜10 cm² area represented treatment of the majority of the UVB damaged skin and may also remove most of the immunosuppressive cells. The behavior of these cells is poorly understood, but they may be able to migrate and/or set up a locally dispersed immunosuppressive environments that promote lesion development (Gabrilovich and Nagaraj, 2009; Stumpfova et al., 2010). Removal of say 20-40% of these cells by treating a ˜4 cm² area may conceivably be insufficient to affect significantly the immunosuppressive environment, especially given that the area after treatment is reduced by ˜3 fold and most of the skin neighbouring the treatment area is also UVB damaged. When considering the ˜10 cm² area, much of the skin neighbouring the treatment area has received less UVB. In this scenario the removal of the immunosuppressive cells may be equally as important for PEP005 gel field treatment as the removal of p53+ lesions (2) The small area that remains after treating a ˜4 cm² area may mean that a clear delineation between lesions arising from inside and outside the tattooed area may be difficult, with p53+ cells originating outside the treatment area potentially growing or migrating into the treatment area during the wound healing process after 0.05% PEP005 gel treatment. During wound healing, keratinocytes are shown to migrate (Natarajan et al., 2006). (3) It is assumed that the reduction of p53 patches+ within the treatment area per cm² is similar when ˜4 or ˜10 cm² area is treated. This would appear likely, but was not demonstrated. Mounting evidence in the literature supports the notion that cutaneous tumours arise from stem cells within skin (Ambler and Maatta, 2009; Gerdes and Yuspa, 2005). Two main groups of skin stem cells have been described in the literature. (a) Stem cells interspersed throughout the basal layer, located in the interfollicular epidermis (IFE). These stem cells are located at the centre of so-called epidermal proliferation units (EPU). (b) Stem cells located within the hair follicle in a well-protected area called the “bulge”. Both populations of stem cells have been implicated as the cells that give rise to cutaneous lesions after UVB irradiation (de Gruijl and Rebel, 2008; Gerdes and Yuspa, 2005), although those arising from the hair follicle bulge appear to have an increased malignant potential (Morris et al., 2000). Recently, evidence supports the notion that interfollicular p53+ patches arise from p53+ stem cells located in the EPU (de Gruijl and Rebel, 2008). The reduction in the number of p53+ patches following 0.05% PEP005 gel treatment may thus reflect the reduction of mutated EPU stem cells. In contrast, the UVB-damaged stem cells located within the hair follicle bulge may be less accessible to 0.05% PEP005 gel field treatment and may be less readily detected by the staining technique used. Conceivably, treatment of the ˜10 cm² area may remove more of the mutated cells, and in particular may remove more of the mutated cells in the hair follicle bulge.

This study suggests that prophylactic PEP005 field treatment is effective in preventing skin cancer development when, for example, a sizable area (˜10 cm²) is treated but may be less effective when a smaller area is treated (˜4 cm²). This may mean that either treating a relatively large area is important for prophylactic efficacy or that treating a large percentage of photodamaged skin in any particular area is important.

There is no difference in growth or size distribution of lesions after PEP005 gel or placebo gel treatment indicating that tumors emerging after 0.05% PEP005 gel treatment do not behave more aggressively.

### 3.2 PEP005 for Tattoo Removal

Treatment with 0.05% PEP005 gel appears to lead to the removal of some of the tattooed lines suggesting that PEP005 gel is useful in tattoo removal.

### 3.3 Spot Therapy of UVB-Induced Lesion

Spot-therapy of UVB-induced lesions within a severely photodamaged area with high dose PEP005 gel (0.1-0.25%) did not lead to discernable cures of any of the lesions treated. However, partial responses following treatment with PEP005 gel did appear to occur (Table 1). A major problem with this model is the difficulty in determining the effect of treatment of a single lesion when multiple lesions emerge within the same and/or surrounding areas. This model would thus appear largely unsuitable for evaluating the efficacy of topical PEP005 gel for tumour therapy.

### Example 5

In accordance with this Example 5, it shows that about 70% of T7 tumours (established in female SKH1/hr mice) are cured after topical treatment, daily for 2 days with 0.25% ingenol mebutate gel. The cure rate in male mice is about 30%, although differences between the sexes did not reach statistical significance. Treatment of T7 tumours ias associated with hemorrhage at the treatment site and is accompanied by a neutrophil infiltrate. Transmission electron microscopy analysis shows that at one hour post-treatment the following could be seen; (i) swelling of mitochondrial cristae in cancer cells, (ii) extravasation of neutrophils and (iii) signs of hemorrhage. Further disruption of the inner mitochondrial membranes in cancer cells is seen at 6 h post-treatment, and tumour cell death by primary necrosis is clearly evident by 24 h. Ingenol mebutate treatment leads to the generation of anti-T7 antibodies, which appear to decline over time. Mice that are cured of T7 tumours by topical ingenol mebutate treatment do not show increased resistance to a subsequent challenge with T7 cells.

### 1. Materials and Methods:

1.1 Materials

1.1.1 Cell Lines

Murine SCC cell lines UV-13.1 and T7 are provided by Dr G. Halliday (University of Sydney, NSW, Australia). Murine B16 cells are provided by Prof Peter Parsons (QIMR, QLD, Australia). All cell lines are negative for mycoplasma infection. Both T7 and UV-13-3 cell lines also test negative for mouse hepatitis virus, minute virus of mice, mouse parvovirus and rotavirus.

1.1.2 Description of Test Systems

Inbred C3H/HeN, inbred SKH1/hr, are obtained as follows: inbred C3H/HeN mice are imported from Charles River Laboratory, NCI Frederick, USA. A breeding colony is established at QIMR; inbred SKH1/hr mice are imported from ARC, Perth, Australia. A breeding colony is established at QIMR. Mice used in the studies are over 4 weeks old at time of inoculation.

1.1.3 Test and Control Articles

Drug Product—Ingenol mebutate
0.1% PEP005
0.25% PEP005
Placebo gel
Samples are stored at 4° C. at QIMR.

API-Ingenol mebutate (PEP005, #250803) is dissolved in acetone and aliquoted into 1 mg aliquots that are dried down and stored at 4° C.

1.1.4 Test and Control Articles Administration

Mice received 20-30 μl of 0.1-0.25% (w/v) gel applied daily for two days via topical route of administration.

1.2 Methods

1.2.1 Culture of Cell Lines

UV-13.1 and T7 cells are cultured in DMEM medium (Gibco Catalogue number 11960) supplemented with sodium-pyruvate (Gibco Catalogue number 11360-070, final concentration 1 mM), L-glutamine (Gibco, catalogue number 25030-081, final concentration 2 mM) and penicillin/streptomycin (Catalogue number 490269, diluted 1:100). B 16 cells are cultured in RPMI (QIMR) supplemented with 10% FBS and penicillin/streptomycin.

1.2.2 In Vitro Cytotoxicity Assay

Cells are seeded with in 10,000 per microtitre wells in 100 μl of culture medium and incubated overnight to enable them to adhere to the bottom of the well. Quadruplicate wells aere treated with ingenol mebutate (PEP005 #250803 final concentration 0.232-232 μM) dissolved in DMSO (Sigma, D2650). The concentration of the vehicle does not exceed 1% v/v and drug dilutions are made in DMEM. Included in the assay were cells treated with vehicle alone (1% DMSO) and control. The treated cells are incubated at 37° C. for 48 h. Medium from the microtitre plates is tipped out; the wells are washed once with PBS to remove any excess FBS and then are fixed with methylated spirits for a minimum of 5 min. The methylated spirit is tipped off and the wells are washed once with tap water. A volume of 50 μl of sulforhodmain B (SRB) solution (0.4% SRB in 1% acetic acid) is added to each well and the plates are incubated at room temperature for a minimum of 15 min. The SRB solution is tipped off and the plates are washed twice with tap water and then twice with 1% acetic acid. The final wash of acetic acid is tapped out of the plate and 100 μl of 10 mM Tris base (unbuffered, pH>9) is added per well. The plate is incubated at room temperature for at least 5 min and then the absorbance is read on an ELISA plate reader at 564 nm.

1.2.3 Inoculation of Mice with Tumours

Groups of hairless inbred SKH1/hr mice are injected s.c. on the dorsum with tumour cells (2×10⁴2×10⁶) harvested from exponentially growing cultures. Tumour development is monitored by visual examination and measurements of the tumour length and width are taken using digital callipers. The tumour size is represented as an area calculated as the tumour width×length as measured on the living animal. In accordance with the QIMR animal ethics committee (AEC) mice are sacrificed when tumours reached an area of 100 mm².

1.2.4 Topical Treatment of Tumour

Ingenol mebutate or vehicle are dispensed over the tumour area using a pipette and then spread using the pipette tip.

1.2.5 Monitoring of Tumour Site

After treatment, lesions were monitored for tumour size (mm²), local tumour reoccurrence and eschar formation. Measurements were made using digital callipers.

1.2.6 Histology Analysis of Tumour Samples

Samples are processed and stained by QIMR Histotechnology.

1.2.7 Transmission Electron Microscopy (TEM)

Samples are processed for TEM by QIMR Histotechnology. Staining and photography is performed at the Royal Brisbane and Women's Hospital (RBWH) Anatomical Pathology Department.

1.2.8 T7 ELISA

Blood (˜50-100 μl) 1 s taken by tail bleeding the mice and is collected into 0.8 ml MiniCollect® Z Serum Separator tubes that are coated with spray-dried clot activator (Greiner bio-one Cat. Num. 450472) and are spun at 3000 rpm in a microfuge at room temperature for 10 min. The serum is collected and stored at 4° C. for analysis of anti-T7 antibodies by ELISA.

Preparation of T7 ELISA plates: T7 cells are grown to 90% confluence. The medium is aspirated and the cells are washed once with ice-cold PBS. T7 cells are harvested by scraping in PBS supplemented with protease inhibitor cocktail (PIC) and disrupted by sonication at 4° C. for 1-2 min. The T7 lys ate solution is supplemented with bicarbonate buffer (pH 9.6) containing PIC to a final volume of 40 ml and 70 μl/well is added to each well in a 96 well plate (Nunc, Invitro Technologies Cat. Num. 442404). The plates are dried overnight at 37° C.

The following day, the plates are blocked with 10% (w/v) skim milk in PBS for 1 h at room temperature. Meanwhile mouse serum is prepared by diluting 1:7 to 1:413343 in 3-fold dilutions in PBS/0.01% Tween20 (v/v)/1% (w/v) skim milk for the IgG2a plate and 1:10 10 1:590410 in 3-fold dilutions for the IgG1 plate. The blocking buffer is discarded from the wells, the plate is washed with PBS/(0.01%) Tween20 and 70 μl of pre-diluted mouse serum is added to duplicate wells. The ELISA plates are incubated at room temperature for 2 h. The plates are then washed 3 times with PBS/(0.01%) Tween20. After the final wash, 100 μl/well of Biotin-conjugated rat anti-mouse antibody IgG2a (BD Pharmingen™ Cat. Num. 553388) or IgG1 (BD Pharmingen™ Cat. Num. 553441) diluted 1:5000 (IgG2a) or 1:3000 (IgG1) in PBS/0.01% Tween20 is added and the ELISA plate is incubated at room temperature for 45 min. The plate is washed 3 times in PBS/(0.01%) Tween20 and 100 μl of streptavidin-HRP (Bioscource, Cat. Num SNN4004) diluted 1:10000 in PBS/0.01% Tween20 is added to each well and is incubated at room temperature for 45 min. The plate is washed a further 3 times in PBS/(0.01%) Tween20, and then is developed with 100 μl/well of solution containing 2,2′-Azino-bis(3-ethlbenzothiazoline-6-sulfoniate) (ABTS, Sigma Cat. Num. 697K41061, 10 mg/ml):(30%) H2O2 (Reidel de Haer, Cat Num. 18312) (1000:1) for 30 min at room temperature. Spectrophotometric readings are taken on an ELISA plate reader at 405 nm and graphs are generated using Graphpad Prism Version 5.01 software.

### 2. Results

2.1 In Vitro Cytotoxicity Profile of Ingenol Mebutate

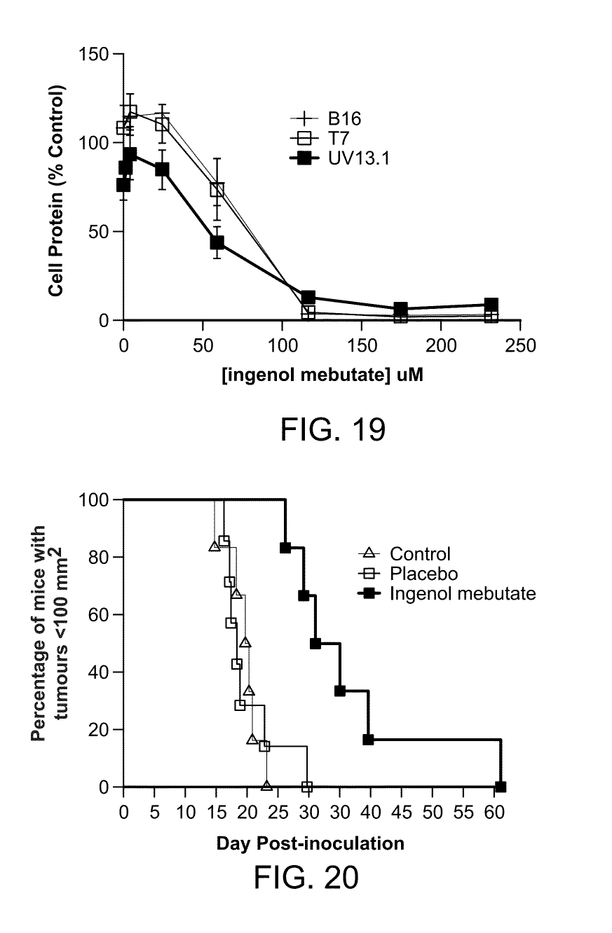

To determine the in vitro sensitivity profile of T7 cells to ingenol mebutate treatment, a dose-response is generated by using the SRB stain to monitor cellular protein. Cells are seeded at high density and treated with ingenol mebutate (0.232-232 μM) for 48 h. The plates are fixed and are stained with SRB. For comparative analysis, another UV-induced murine SCC cell line, UV-13.1, and the murine melanoma cell line, B16, cells are included in the assay. FIG. 19 shows the dose-response curves for the three cell lines. The data is presented as the percentage of untreated control cell protein. All three cell lines showed a similar sensitivity profiles to ingenol mebutate. The 90% lethal dose (LD90) for all three cell lines (including B16) is very similar and in the 100-110 μM range. Previously, LD90 values, of 180-220 μM are reported, with B16 showing an LD90 of 190 μM (Ogboume et al. 2004). The discrepancy may arise from (i) different culture parameters (e.g. seeding density, fetal calf serum), (ii) different procedures for measuring ingenol mebutate concentrations and/or (iii) the use of DMSO to dissolve ingenol mebutate rather than the acetone used in the Ogboume et al. study (2004).

FIG. 19 shows an in vitro dose response for acute cell cytotoxicity of ingenol mebutate. Cells are treated with 0.232-232 μM of ingenol mebutate. Vehicle treated cells (0 ug/ml ingenol mebutate) and untreated control cells are included in the assay. After 2 days, total protein is determined using SRB staining. Data are presented as percentage of untreated control cell protein. The results represent the mean and SEM of three independent experiments.

2.2 Determining the Curative Regimen of Ingenol Mebutate in the T7/SKH1 Model of SCC

Female inbred SKH1/hr mice are injected on their dorsa on Day 0 s.c. with 2×10⁶ T7 cells in 100 μl of medium. Four days later the mice are segregated into 3 groups so that the mean tumour area and the range of tumour sizes is approximately similar between the groups (Table 2). The groups are treated topically with 20 μl of 0.1% ingenol mebutate or placebo gel daily for 2 days. Control mice remain untreated. Tumour growth is monitored over time. Mice are excluded from the study if they developed intra-muscular tumours as this indicated that the tumours are injected too deep. In this study, one control and one ingenol mebutate treated mice are excluded for this reason. Mice are euthanased when the tumours reached 100 mm². Table 2 summarizes the information for the mice cohort within the study, omitting mice that are excluded for the above stated reason. Survival curves are generated by attributing a death event when a tumour reached 100 mm². The results depicted in FIG. 20 show that treatment with about 0.1% ingenol mebutate daily for two days led to a significant delay in tumour growth (Log-rank test placebo vs. ingenol mebutate p=0.0029). However, none of the tumours are cured. Past experience suggested that the tumours may have been too large at the time of treatment for ingenol mebutate to mediate tumour clearance.

TABLE 2
Topical treatment of s.c. T7 tumours with 0.1% ingenol mebutate in
female mice

| | | | Ingenol |
|---|---|---|---|
| | Control | Placebo | mebutate |
| Gender | Female | Female | Female |
| N/group | 6 | 7 | 6 |
| Mean Tumour Area (mm²) | 17.1 | 15.8 | 16.8 |
| Tumour Area Range (mm²) | 12-25 | 10-24 | 9-28 |

FIG. 20 shows treatment of T7 tumours in inbred female SKH1/hr mice with 0.1% ingenol mebutate daily for 2 days. Mice aere inoculated on Day 0 s.c. with 2×10⁶ T7 cells on their dorsa. On days 4 and 5, the tumours are treated topically with 20 μl of 0.1% ingenol mebutate. Tumour area is monitored over time. The mice are sacrificed when the tumours reached 100 mm². Survival curves are generated by attributing a death event when a tumour reached 100 mm². Log-rank test placebo vs. ingenol mebutate p=0.0029.

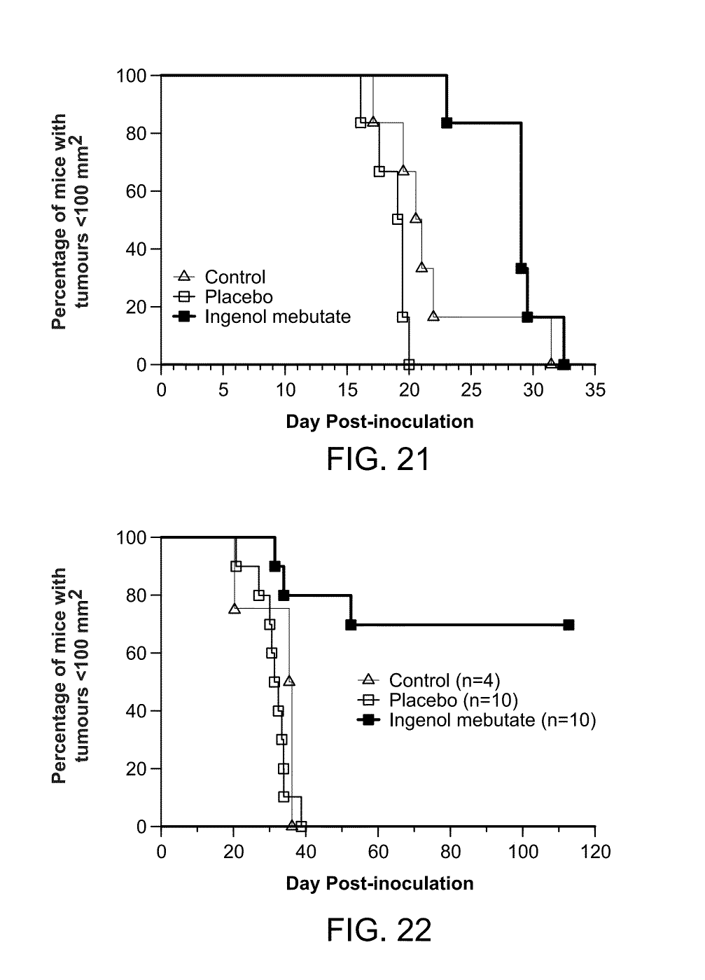

In an attempt to improve cure rates in this model, smaller T7 tumours are treated with 0.1% ingenol mebutate. In addition, the inoculation volume is reduced to about 50 μl to reduce the spread of tumour cells beyond the inoculation site. Female inbred SKH1/hr mice are injected on day 0 s.c. with 5×10⁵ T7 cells (one quarter of the cell number used above) in about 50 μl of medium on their dorsa. Three days later the mice are segregated into 3 groups so that the mean tumour area and range is approximately similar between groups (Table 2). The groups are then treated topically with about 20 μl of 0.1% ingenol mebutate or placebo gel, daily for 2 days. Control mice remained untreated. Tumour growth is monitored over time. Mice are sacrificed when the tumours reached 100 mm². Table 3 summarises the information for the mice in this cohort. Treatment with 0.1% ingenol mebutate daily for two days led to a significant reduction in tumour growth (Log-rank test placebo vs ingenol mebutate p=0.0006) (FIG. 21). However, significance was not achieved when comparing control vs ingenol mebutate treated tumour (Log-rank test placebo vs ingenol mebutate p=0.06), due to one slow-growing tumour in the control group. No cures are obtained even though the tumours are smaller at the time of treatment. This data indicated that the reduced tumour size did not significantly affect cure rates after treatment with 0.1% ingenol mebutate.

TABLE 3
Topical treatment of T7 tumours with 0.1% ingenol mebutate in
female SKH1/hr mice

| | | | Ingenol |
|---|---|---|---|
| | Control | Placebo | mebutate |
| Gender | Female | Female | Female |
| N/group | 6 | 6 | 6 |
| Mean Tumour Area (mm²) | 10.57 | 10.19 | 12 |
| Tumour Area Range (mm²) | 4-13 | 4-12.5 | 9-14 |

Mice are inoculated s.c. with 5×10⁵ T7 cells on their dorsa. On days 3 & 4, the tumours are treated topically with 20 μl of 0.1% ingenol mebutate. Tumour area is monitored over time. The mice are sacrificed when the tumours reach 100 mm². Survival curves are generated by attributing a death event when a tumour reached 100 mm². Log-rank test placebo vs ingenol mebutate p=0.0006.

Four changes are adopted to try and achieve cure with ingenol mebutate treatment in this model: (i) the dose of ingenol mebutate is increased to 0.25%; (ii) the injection depth of T7 cells is reduced to try and achieve a shallow subcutaneous inoculation; (iii) the T7 cell numbers are further reduced to 5×10⁴ T7 cells; and (iv) the volume of gel applied is increased from 20 μl to 30 μl to ensure that the gel is spread around the entire tumour site. Female SKH1/hr mice are inoculated by shallow injection with 5×10⁴ T7 cells in 30 μl (Study 1) or 50 μl (Study 2) on day 0. On day 4 the mice are segregated into 3 groups, on days 4 & 5 the mice are treated with either 30 μl of 0.25% ingenol mebutate or placebo gel. A control group remain untreated. By day 4, the tumours reach a similar mean size (Table 4) as those described above (Table 3). Tumour growth is monitored over time. Mice are euthanased when the tumours reach 100 mm². Table 4 shows the combined data from two independent studies using the four adopted changes. Both studies show very similar results and data is thus combined. Survival curves in FIG. 22 are generated by attributing a death event when the tumour area reached 100 mm².

Topical treatment of T7 tumours with 0.25% ingenol mebutate result in a significant reduction in the number of mice with tumours reaching 100 mm² (Log-rank test placebo vs ingenol mebutate, p=0.0002), with 70% of mice showing no signs of tumour 4 months after treatment, suggesting that complete cure is achieved.

TABLE 4
Topical treatment of T7 tumours with 0.25% ingenol mebutate in
female mice

| | | | Ingenol |
|---|---|---|---|
| | Control | Placebo | mebutate |
| Gender | Female | Female | Female |
| N/group | 4 | 10 | 10 |
| Mean Tumour Area (mm²) | 11 | 10.8 | 10.5 |
| Tumour Area Range (mm²) | 7-13.7 | 4-17.5 | 9-12.6 |

FIG. 22 shows treatment of T7 tumours in inbred female SKH1/hr mice with 0.25% ingenol mebutate daily for 2 days. Mice are inoculated (shallow s.c.) with 5×10⁴ T7 cells on their dorsa. On days 4 & 5, the tumour sites are treated topically with 30 μl of 0.25% ingenol mebutate. Tumour areas are monitored over time. The mice are sacrificed when the tumours reached 100 mm². Survival curves are generated by attributing a death event when a tumour reached 100 mm². Log-rank test placebo vs ingenol mebutate, p=0.0002. The data for ingenol mebutate and placebo groups is derived from two independent studies, whereas the control group is only included in one study.

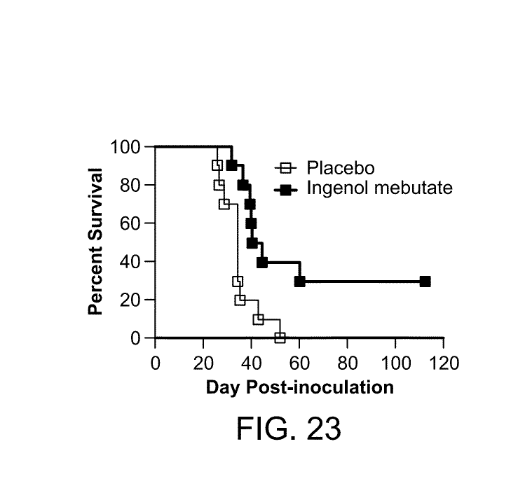

To determine whether male mice bearing T7 tumours are also cured by the same ingenol mebutate regimen, we conduct similar studies using male inbred SKH/hr1 mice. Male SKH1/hr mice are inoculated by shallow injection with 5×10⁴ T7 cells in 30 μl on Day 0. On day 4, the mice are segregated into 3 groups and on days 4 & 5 the mice are treated with either 30 μl of 0.25% ingenol mebutate or placebo gel. Tumour growth is monitored over time. Mice are euthanased when the tumours reached 100 mm². One ingenol mebutate treated mouse is found deceased due to unknown reasons and is therefore excluded from the study. Table 5 summarizes the information for cohorts of mice from 2 independent studies, which show similar results. Survival curves in FIG. 23 are generated by attributing a death event when the tumour area reaches 100 mm².

Topical treatment of T7 tumours with 0.25% ingenol mebutate in male SKH/hr mice result in a significant reduction in the number of mice with tumours reaching 100 mm² (Log-rank test placebo vs ingenol mebutate, p=0.0084), with 30% of mice showing no signs of tumour reoccurrence 4 months after treatment, suggesting that complete cure is achieved.

TABLE 5
Topical treatment of T7 tumours with 0.25% ingenol mebutate in
male mice

| | | Ingenol |
|---|---|---|
| | Placebo | mebutate |
| Gender | Male | Male |
| N/group | 10 | 10 |
| Mean Tumour Area (mm²) | 11.2 | 11.2 |
| Tumour Area Range (mm²) | 5.2-18.5 | 5-18 |

FIG. 23 shows treatment of T7 tumours in inbred male SKH1/hr mice with 0.25% ingenol mebutate daily for 2 days. Mice are inoculated (shallow s.c.) with 5×10⁴ T7 cells on their dorsa. On days 4 & 5, the tumour sites are treated topically with 30 μl of 0.25% ingenol mebutate. Tumour areas are monitored over time. The mice are sacrificed when the tumours reached 100 mm². Survival curves are generated by attributing a death event when a tumour reaches 100 mm². Log-rank test placebo vs ingenol mebutate, p=0.0084. The graphs shows data from two independent studies, which showed similar results.

2.3 No Statistical Significance Between Male and Female Cure Rates

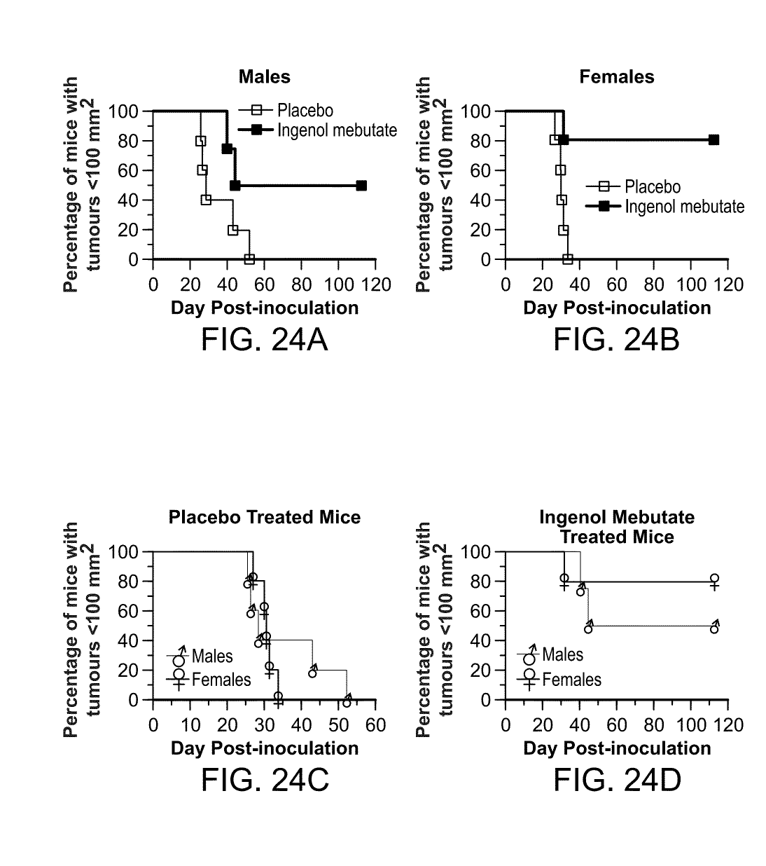

To determine if there is any statistical difference in cure rates between male and female SKH1/hr mice two analyses are undertaken. Firstly, cure rates are compared from a study where male and female mice are inoculated from the same pool of T7 cells and treated with ingenol mebutate or placebo gel at the same time. (Each of these studies represented one of the two experiments combined to produce the data shown in 2.2). Table 6 summarizes the information for this cohort of mice. Topical treatment of T7 tumours in male mice leads to a reduction in tumour growth, although this does not reach statistical significance (log-rank test placebo vs. ingenol mebutate p=0.0834) (FIG. 24A). In male mice cure is achieved in about 50% of treated mice ( 2/4) (FIG. 24A). Topical treatment of T7 tumours in female mice with ingenol mebutate leads to a significant reduction in tumour growth as compared to placebo treated tumors with complete cure achieved in about 80% of female mice (Log-rank placebo vs. ingenol mebutate p=0.0127) (FIG. 24B). Comparisons of male vs. female mice after placebo treatment (FIG. 24, panel C) or 0.25% ingenol mebutate treatment (FIG. 24, panel D) showed no statistical significance.

TABLE 6
Topical treatment of T7 tumours in male and female inbred SKH1
mice with 0.25% ingenol mebutate

| | | Ingenol | | Ingenol |
|---|---|---|---|---|
| | Placebo | mebutate | Placebo | mebutate |
| Gender | Male | Male | Female | Female |
| N/group | 5 | 4 | 5 | 5 |
| Mean Tumour Area (mm²) | 10.9 | 11.3 | 11.5 | 10.7 |
| Tumour Area Range (mm²) | 7.5-12.3 | 9-13 | 10.3-13.2 | 9-13.5 |

FIG. 24 shows the effects of gender on ingenol mebutate cure rates in SKH1/hr mice, results from a direct comparative study. Mice are inoculated (shallow s.c.) with 5×10⁴ T7 cells on their dorsa. On days 4 & 5, the tumour sites are treated topically with 30 μl of 0.25% ingenol mebutate. Tumour area is monitored over time. The mice are sacrificed when the tumours reached 100 mm². Survival curves are generated by attributing a death event when a tumour reached 100 mm². A. Male mice (Log-rank test, placebo vs ingenol mebutate p=0.08). B. Female mice (Log-rank test placebo vs ingenol mebutate p=0.0127). C. Placebo treated male vs female mice (Log-rank test p=0.64). D. Ingenol mebutate treated male vs female mice (Log-rank test, p=0.47).

In an alternative analysis of the results, combined data from males vs. females from Tables 4 and 5, and FIGS. 24 & 25 are compared. Table 7 summarizes the information for the combined cohorts of mice. Although the cure rate in female mice is about 70%, compared to about 30% in males, this did not reach statistical significance (Log-rank test male vs. female p=0.108).

TABLE 7
Topical treatment of T7 tumours in male and female inbred
SKH1 mice with 0.25% ingenol mebutate

| | Ingenol | Ingenol |
|---|---|---|
| | mebutate | mebutate |
| Gender | Female | Male |
| N/group | 10 | 10 |
| Mean Tumour Area (mm²) | 10.5 | 11.2 |
| Tumour Area Range (mm²) | 9-12.6 | 5-18 |

FIG. 25 shows the effect of gender on ingenol mebutate cure rates in SKH1/hr mice, results from two independent studies. Mice are inoculated (shallow s.c.) with 5×10⁴ T7 cells on their dorsa. On Days 4 & 5, the tumour sites were treated topically with 30 μl of 0.25% ingenol mebutate. Tumour area is monitored over time. The mice are sacrificed when the tumours reached 100 mm². Survival curves are generated by attributing a death event when a tumour reached 100 mm². The data represents the results from two independent studies for both male and female mice. Log-rank test male vs female, p=0.108.

2.4 Appearance of Ingenol Mebutate Gel Post Topical Application on T7 Tumours In Vivo

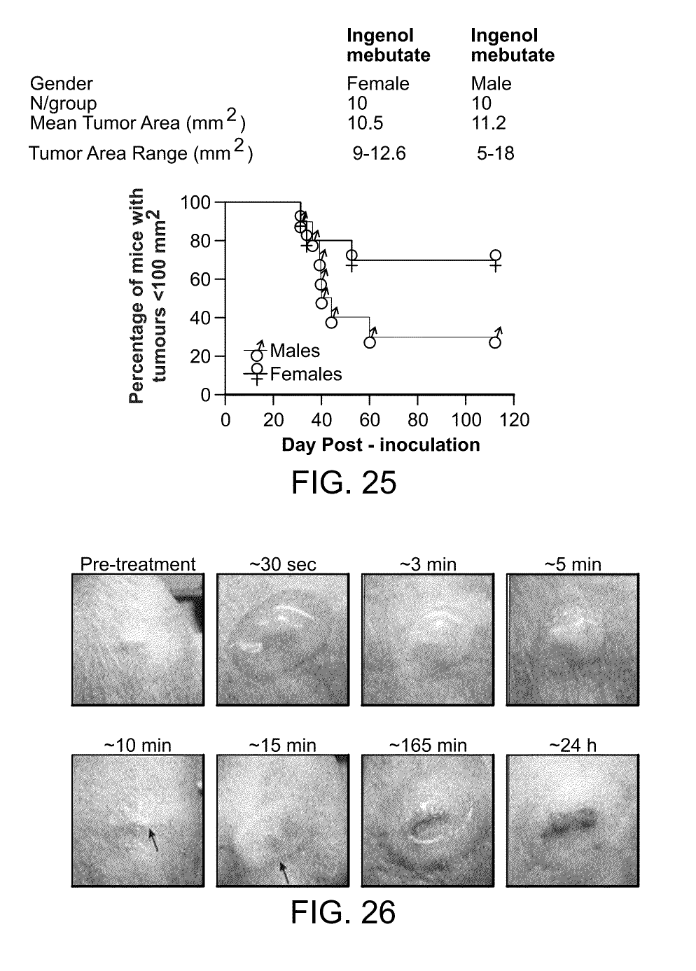

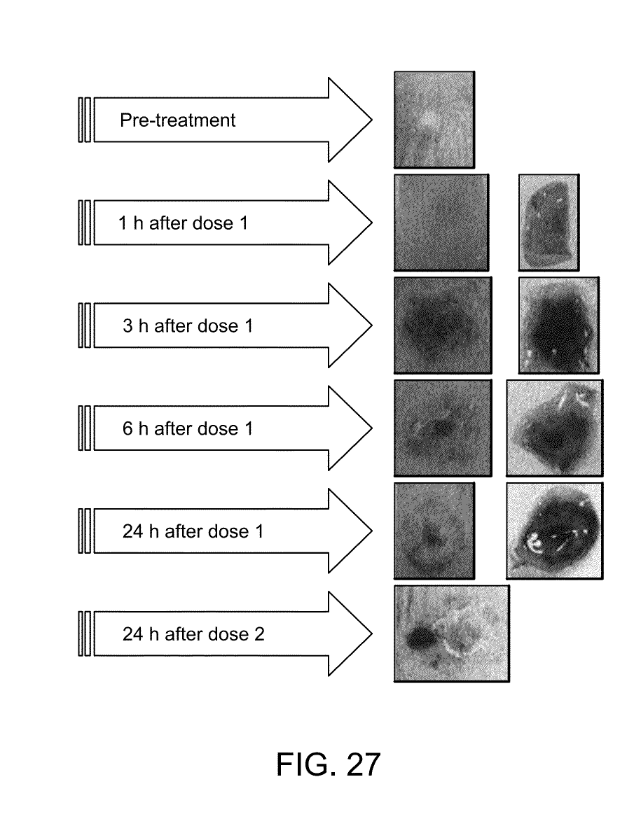

Visual examination of the gel formulation following treatment with 0.25% ingenol mebutate (FIG. 26) shows that the gel goes from clear to opaque within 3 min. By 10 min the gel is no longer clearly visible and a small white spot remained on the skin (FIG. 26. 10 mins, arrow). A slight blistering is evident 15 min after treatment (FIG. 26. 15 mins., arrow) and hemorrhaging is visible ˜165 min after treatment (FIG. 26. ˜165 min) Twenty-four hours following application of 0.25% ingenol mebutate, a dark red/black area appears to be largely confined to the tumour area (FIG. 27. ˜24 h).

FIG. 26 shows the appearance of ingenol mebutate gel post topical application on T7 tumors. Mice are inoculated T7 cells on their dorsa. On day 4, the tumours are treated topically with 30 μl of 0.25% ingenol mebutate. Photographs are taken at the indicated time-points. Arrow at 10 min points at white spot. Arrow at 15 min points at ingenol mebutate induces blistering.

2.5 Visual & Histological Examination of T7 Tumours Treated Topically with 0.25% Ingenol Mebutate

To determine the effects of topical treatment of T7 tumour with 0.25% ingenol mebutate, we conducted a photographic and histological time-course analysis. Mice are inoculated with T7 cells on their dorsa. On days 4 & 5, the tumours are treated topically with 30 μl of 0.25% ingenol mebutate. The photographs in FIG. 27 show that 1 h after ingenol mebutate treatment there is a slight reddening of the treated area as seen when viewing the skin epidermis side up. After 3 h post-treatment haemorrhage is evident and extensive, encompassing the whole treated area, as indicated in the skin photographed dermis side up. By 6 h treatment the hemorrhaging appears to be more localized to the tumour site and this is maintained at 24 h after the first and second dose. A green tinge to the treatment site is evident by 24 h after dose 1 and more apparent 24 h after dose 2.

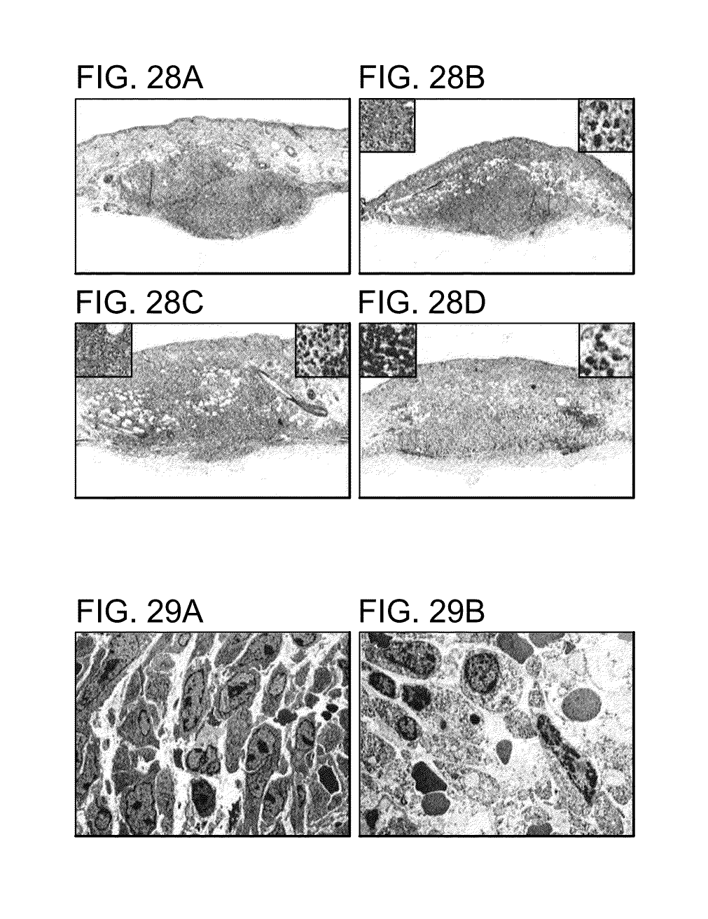

Histology analysis (FIG. 28) confirms that hemorrhage is present 3 h after topical treatment with one dose of 0.25% ingenol mebutate treatment (FIG. 28, panel B) and is extensive 24 h after one dose (FIG. 28, panel D). Hemorrhage appears to be extensive in areas surrounding the tumour (FIG. 28, panel C and D).

Concomitant with the haemorrhage is an infiltration of polymorphonuclear cells (probably neutrophils) that were evident 3 h after treatment with ingenol mebutate (FIG. 27, panel B) They appear to increase in number by 6 h post-treatment (FIG. 27, panel C). They are still present, although reduced in numbers, by 24 h post-treatment. Twenty four hours after 0.25% ingenol mebutate treatment (FIG. 9, panel D) most of the cells appear to be dead with very dark staining nuclei, making it difficult to identify the cell type.

FIG. 27 shows images of T7 tumour sites post ingenol mebutate treatment. Mice are inoculated T7 cells on their dorsa. On days 4 & 5, the tumours are treated topically with 30 μl of 0.25% ingenol mebutate (dose 1 & 2, respectively). Photographs are taken at the indicated time-points, before and after dose 1 and dose 2. After 1, 3, 6, and 24 h post ingenol mebutate treatment the mice are sacrificed and the treated area excised. The photographs on the right show excised tumour sites with the skin, dermal side up.

FIG. 28 shows H&E staining of in vivo T7 tumours treated with 0.25% ingenol mebutate. Mice are inoculated T7 cells on their dorsa. On day 4 the tumours are treated topically with 30 μl of 0.25% ingenol mebutate. At indicated time-points the mice are sacrificed, the tumour excised and processed for H&E staining. A. Placebo treated. B. 3 h 0.25% ingenol mebutate. C. 6 h ingenol mebutate. D. 24 h ingenol mebutate. Insets in B, C and D are high magnifications showing haemorrhaging on the left-hand side and infiltrating polymorphonuclear leucocytes (probably neutrophils) on the right-hand side.

2.6 Subcellular Changes Induced in T7 Tumour Cells Topically Treated with 0.25% Ingenol Mebutate

Previously TEM analysis of B 16 melanoma tumours are treated in vivo show early mitochondrial swelling (6 h post-treatment) and death by necrosis as key events in the chemoablative actions following topical treatment with ingenol mebutate (Ogbourne et al. 2004). In this study, we conduct a time-course analysis to determine the effects of topical treatment of T7 tumours with 0.25% ingenol mebutate. Female inbred SKH1/hr mice bearing T7 tumours are inoculated by shallow s.c. injection are topically treated with one dose of 0.25% ingenol mebutate or placebo gel. Mice are sacrificed, 1 h, 3 h, 6 h and 24 h post-ingenol mebutate treatment and 24 h post placebo treatment and are analyzed by TEM.

2.6.1 Effect of Ingenol Mebutate on T7 Cells

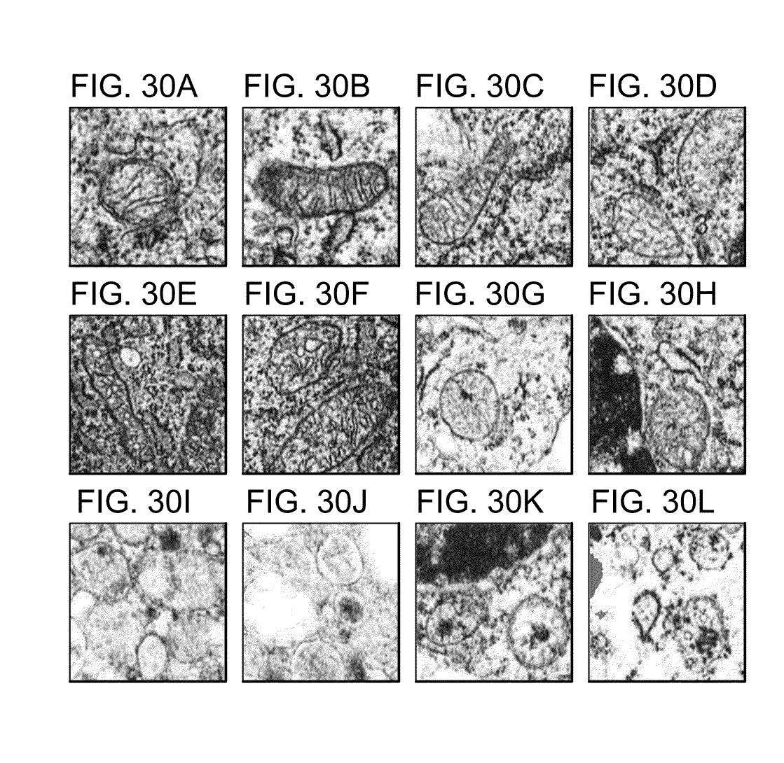

TEM showed that treatment with 0.25% ingenol mebutate results in cell death by primary necrosis and is evident 24 h after treatment (FIG. 29). At 1 h post treatment (FIG. 30, panels C and D) with ingenol mebutate, disruption of the mitochondrial cristae could be seen, which became more extensive by 3 h (FIG. 30, panels E and F). Six hours after treatment (FIG. 30, panels G and H) (in addition to cristae disruption) dense spicules within the mitochondrion, resembling calcium depositions, become apparent. At 16 h the mitochondria showed more extensive degeneration, with cristae structures largely lost (FIG. 30, panels I-L), and the appearance of dense granules within the mitochondrion became more apparent.

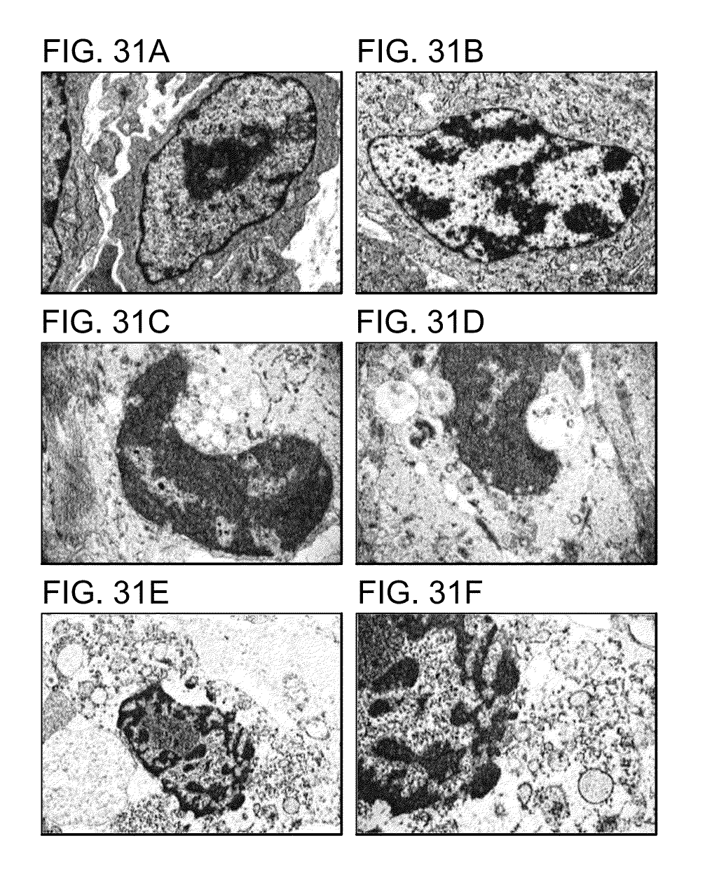

Ingenol mebutate treatment also led to morphological changes to the nucleus (FIG. 31). Six hours post treatment, chromatin within tumour cells appeared to aggregate and become more electron dense (FIG. 31, panel B). By 16 h post ingenol mebutate treatment, the nuclear envelope appeared to be disrupted (FIG. 31, panel C and D) and by 24 h in some tumour cells no nuclear envelope could be detected, with chromatin appearing to be leaking into the cytosol (FIG. 31, panel E and F). At this stage the cytoplasmic structures also become completely disrupted. These morphological analyses suggest tumour cell death is by primary necrosis and are consistent with previous reports (Ogbourne et al. 2004).

FIG. 29 shows primary necrosis induced by ingenol mebutate treatment of T7 tumours. TEM of T7 tumours that are treated in vivo with 0.25% ingenol mebutate or placebo gel. A. T7 tumour is treated for 24 h with placebo gel. B. T7 tumour is treated for 24 h with 0.25% ingenol mebutate.

FIG. 30 shows mitochondrial changes induced in T7 tumours treated topically with ingenol mebutate. TEM of T7 tumours treated in vivo with 0.25% ingenol mebutate or placebo gel. A-B. 24 h placebo gel. C-D, 1 h post 0.25% ingenol mebutate, E-F, 3 h post 0.25% ingenol mebutate. G-H, 6 h post ingenol mebutate I-J, 16 h post ingenol mebutate K-L, 24 h post ingenol mebutate

FIG. 31 show nuclear and cytoplasmic changes induced in T7 tumours grown on SKH1 mice and treated topically with ingenol mebutate. TEM of T7 tumours treated topically with 0.25% ingenol mebutate or placebo gel. A. 24 h placebo gel. B. 6 h post 0.25% ingenol mebutate treatment, C-D. 16 h post 0.25% ingenol mebutate treatment. E-F. 24 h post ingenol mebutate treatment. Panels D and F are enlargements of areas from panels C and E, respectively.

2.6.2 Effect of Ingenol Mebutate on Vasculature and Immunity

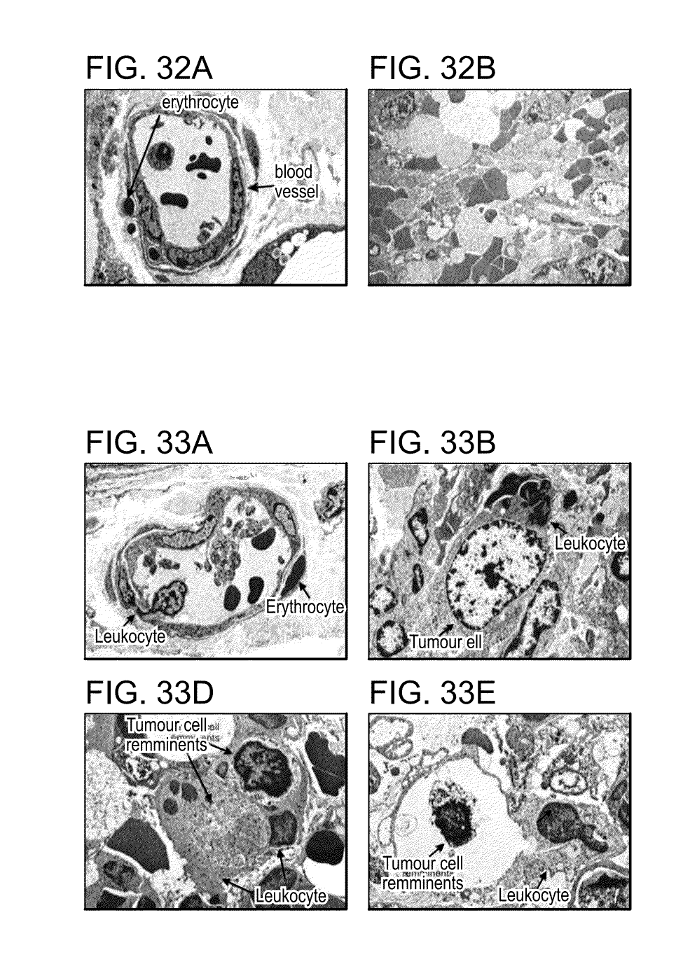

Histological analysis of the T7 tumour that are treated with 0.25% ingenol mebutate show extensive local haemorrhaging and a neutrophil infiltrate as key features following treatment (FIG. 27). Consistent with the histology results, TEM also show haemorrhaging following ingenol mebutate treatment. Extravagating erythrocytes are evident by TEM 1 h post treatment with 0.25% ingenol mebutate (FIG. 32, panel A) and extensive hemorrhage is seen 6 h after treatment (FIG. 32, panel B).

FIG. 32 shows ingenol mebutate inducing haemorrhage. TEM of T7 tumours treated in vivo with 0.25% ingenol mebutate or placebo gel. A. 1 h post 0.25% ingenol mebutate. B. 6 h post 0.25% ingenol mebutate.

Previously reports suggests that neutrophils play an important role in ingenol mebutate-mediated cure of primary tumours by clearing residual tumour cells (Challacombe et al. 2006). The current TEM time-course analysis show that topical treatment of T7 tumours with 0.25% ingenol mebutate leads to extravasation of neutrophils to the tumour site, evident after 1 h (FIG. 33, panel A). At 6 h post treatment, evidence of close interactions between intact tumour cells and polymorphonuclear cells (probably neutrophils) can also be seen (FIG. 33, panel B). By 16 h and 24 h post treatment, phagocytosis of tumour cell remnants by polymorphonuclear cells is apparent (FIG. 33, panel C and D). These data support the view that neutrophils are a major feature of ingenol mebutate treatment and are consistent with the notion that they are involved in the destruction of tumour cells and in the removal of necrotic cellular debris.

FIG. 33 shows polymorphonuclear leukocytes following topical treatment of T7 tumours with ingenol mebutate. TEM of T7 tumours are treated by topical application with 0.25% ingenol mebutate or placebo gel. A. 1 h post 0.25% ingenol mebutate treatment. B. 6 h post 0.25% ingenol mebutate treatment. C. 16 h post 0.25% ingenol mebutate treatment. D, 24 h post ingenol mebutate treatment.

2.7 Generation of Anti-T7 Antibodies Following Cure of T7 Tumours with Ingenol Mebutate

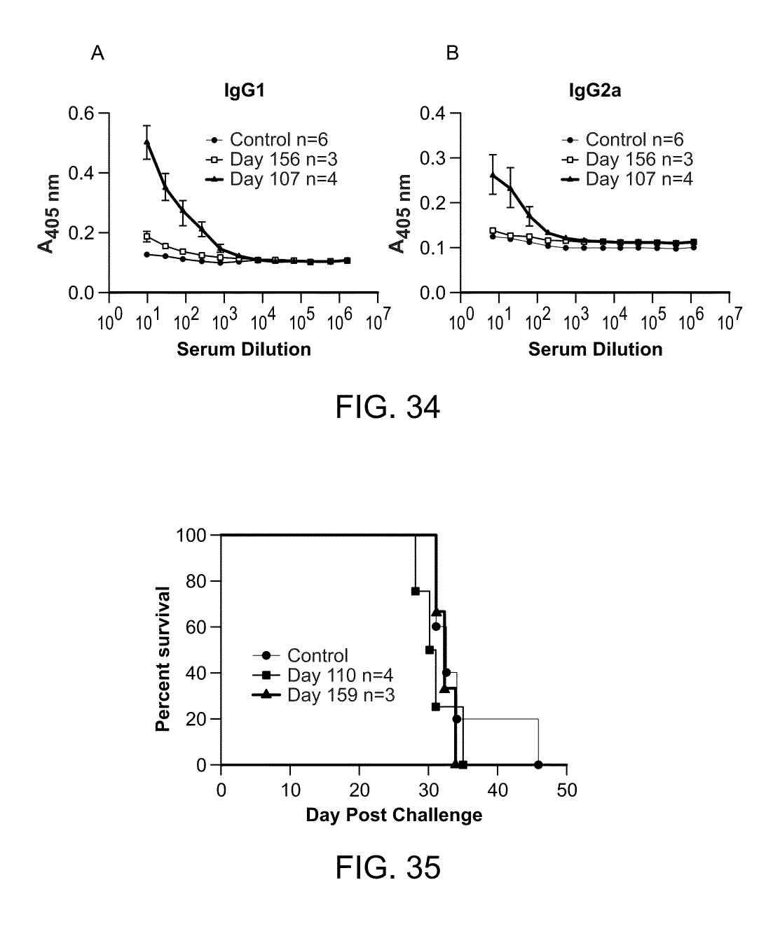

Previously we have shown that ingenol mebutate treated B 16 tumours growing in C57BL/6 mice, lead to increased levels of anti-B 16 antibodies on days 11 and 136 post ingenol mebutate treatment (Challacombe et al. 2006). To determine whether treatment of T7 tumours with ingenol mebutate also led to increased anti-T7 antibodies, an ELISA assay was used to measure the anti-T7 antibodies in (i) female mice cured of T7 tumours with 0.25% topical ingenol mebutate treatment and (ii) in naïve mice bearing no T7 tumours. Ingenol mebutate-cured mice had generated significantly more IgG1 and IgG2a anti-T7 antibodies than naïve mice in groups of mice bled 107 days post treatment (FIG. 34, A and B). A separate group of mice had serum tested on day 156 post treatment, and anti-tumour antibodies were barely detectable (FIG. 34. This might suggest a decline in antibody responses over time, perhaps because no “booster immunisation” was involved. However, it should be noted that these represented two separate groups of mice and so a decline in responses over time was not formally demonstrated.

FIG. 34 shows the measurement of anti-T7 antibodies following cure of T7 tumours with ingenol mebutate. ELISA assays are performed on serum obtained from female mice that had T7 s.c. tumours are cured by topical treatment with 0.25% ingenol mebutate. There are 3 cohorts of mice in the study: control mice have never been treated with ingenol mebutate (Control); Day 156 mice, serum is collected 156 days post ingenol mebutate treatment (Day 156), and Day 107 mice, serum is collected 107 days post-ingenol mebutate treatment. The later two groups ire independent groups of mice, i.e. serum is not taken from the same mice at two time points. A. IgG1 levels. B. IgG2a levels. The data represents the mean and SEM.

2.8 Challenging Ingenol Mebutate Cured Mice with T7 Tumours

We previously reported that cure of large s.c. Lewis lung carcinomas with intratumoural ingenol mebutate treatment resulted in protection against challenge (on day 6 post treatment) with the same tumour at a separate site (Le et al. 2009). To determine if cure of primary T7 tumours in inbred SKH1/hr mice would protect mice from subsequent challenge with the same tumour, T7 cells were injected s.c. in female SKH1/hr mice that had primary T7 tumours previously cured by topical treatment with 0.25% ingenol mebutate. There were 2 groups of mice, mice that had primary tumours cured by ingenol mebutate 110 (group 1) and 159 (group 2) days prior to challenge. Included in the study was a control group that had not previously received T7 or ingenol mebutate treatment. Tumour growth is monitored over time. Mice are culled when tumours reached 100 mm².

Cure of primary T7 tumours with ingenol mebutate have no protective effect on a subsequent T7 challenge. See FIG. 35. Two reasons may account for the differences between this study and the Lewis Lung study (Le et al. 2009); (i) Lewis lung tumours are known to be susceptible to immune based regression in C57Bl/6 mice whereas immune based regression of T7 tumours in SKH1 mice has, to our knowledge, not been demonstrated. (ii) day 6 might be expected to be at or near the peak of effector anti-cancer CD8 T cells levels, whereas by day 110/159 only memory cells would be present, which (unlike effector cells) may have limited activity against tumour cells.

FIG. 35 shows cure of primary tumour with ingenol mebutate does not provide protection against subsequent challenge with T7. Mice are inoculated s.c. with 2×10⁴ T7 cells 110 (Day 110) or 159 (Day 159) days post last treatment of primary tumour with ingenol mebutate. Tumour growth is monitored over time. Mice are sacrificed when tumour reached 100 mm². Survival curves are generated by assigning a death event when tumours reached 100 mm². Control mice had neither prior T7 nor ingenol mebutate treatment.

### 3. Conclusions

This Example 5 describes the efficacy of ingenol mebutate in a murine SCC model, which uses immunologically intact mice. This model might be viewed as a more appropriate model than B16 or LK2 in nude mice for treatment of non melanoma skin cancers in humans. However, the data from the current model parallel those seen in previous mouse models (Challacombe et al. 2006; Le et al. 2009; Ogbourne et al. 2004). Specifically (i) in vitro ingenol mebutate cytotoxicity is similar for T7 and B 16, supporting the view that the in vitro LD90 for most cell lines is quite similar; (ii) ingenol mebutate treatment in vivo results in hemorrhage, mitochondrial disruption in tumour cells, and primary necrosis of tumour cells, (iii) ingenol mebutate treatment results in the recruitment of neutrophils, which may be involved in killing tumour cells, and (iv) ingenol mebutate treatment results in the induction of anti-cancer antibodies. The T7/SKH1/hr model of NMSC has thus provided similar data to that seen previously using other mouse models (which did not use non-melanoma skin cancers and/or used nude mice). After the paper published by Li et al (2010) a greater focus is placed on analysing haemorrhage; however, there is no indication that this is more severe in this T7 model compared with the other models.

In vivo, topical treatment with 0.25% ingenol mebutate of T7 tumours injected shallow s.c. results in cure of about 70% of tumours in female mice.

### Example 6

In accordance with this Example 6, two daily topical applications of ingenol mebutate at about 0.1% or about 0.25% onto tattoos led to the eradication of the tattoo markings.

### 1. Materials and Methods:

1.1 SKH1/hr Model

Male outbred hairless SKH1/hr mice were selected as the test model for this pilot study. Routine animal husbandry was undertaken by the QIMR Bancroft Centre Animal Facility according their standard operating procedures (SOPs). Feed and water was available ad libitum from individual baskets and water bottles attached to the cages.

1.2 Treatments

Ingenol Mebutate gel and placebo gel used in this study.
0.25% PEP005
0.1% PEP005
Placebo Gel

1.3 Tattoo Studies

Fifteen SKH1/hr mice are tattooed on their dorsa with an about 1 cm×about 1 cm cross using human grade blue tattoo ink (Tattoo supplies Australia, Catalogue number FFMB) on Day 0. Once the tattooed area heals (after 12 days), the tattoo (about 100 mm²) is topically treated daily for 2 days with 50 μl of PEP005 gel or placebo gel (Day 12 & Day 13). Two PEP005 doses are tested: 0.1% and 0.25% (w/v). The mice are examined over time to assess any changes to the tattooed area and photographs are taken.

### 2. Results

2.1 PEP005 Treatment Leads to Eradication of Tattoo Markings.

The aim study is to determine whether PEP005 gel can remove tattoo markings when applied topically. Fifteen mice are tattooed with a cross on their dorsa. Twelve days after the tattoos are drawn, they are treated topically daily for 2 days with 0.1% PEP005, 0.25% PEP005 or placebo gel. The photographs in FIG. 1 show the tattoo just prior to the first treatment (Day 12), 24 h after the first treatment (Day 13) and at different time points after the second treatment (from Day 14 onwards). Treatment with a single dose of PEP005 (0.1% or 0.25%) evokes skin edema and hemorrhaging visible 24 h after the first treatment (Day 13). One week after the second dose, an eschar encompassing the whole treatment area forms. This contracts over time and fully resolves after about 3 weeks (Day 32). All of the PEP005 treated tattoos are removed by PEP005 treatment, whereas all of the placebo treated tattoos are still present on Day 232. Six to seven months after the PEP005 treatment is applied, the treated area heals, leaving skin with a slight pink tinge. In some instances, some mild scarring is evident by visual examination (FIG. 2). There is no change in the placebo treated tattoos over this time frame. These data indicate that topical PEP005 is effective as a tattoo removal agent.

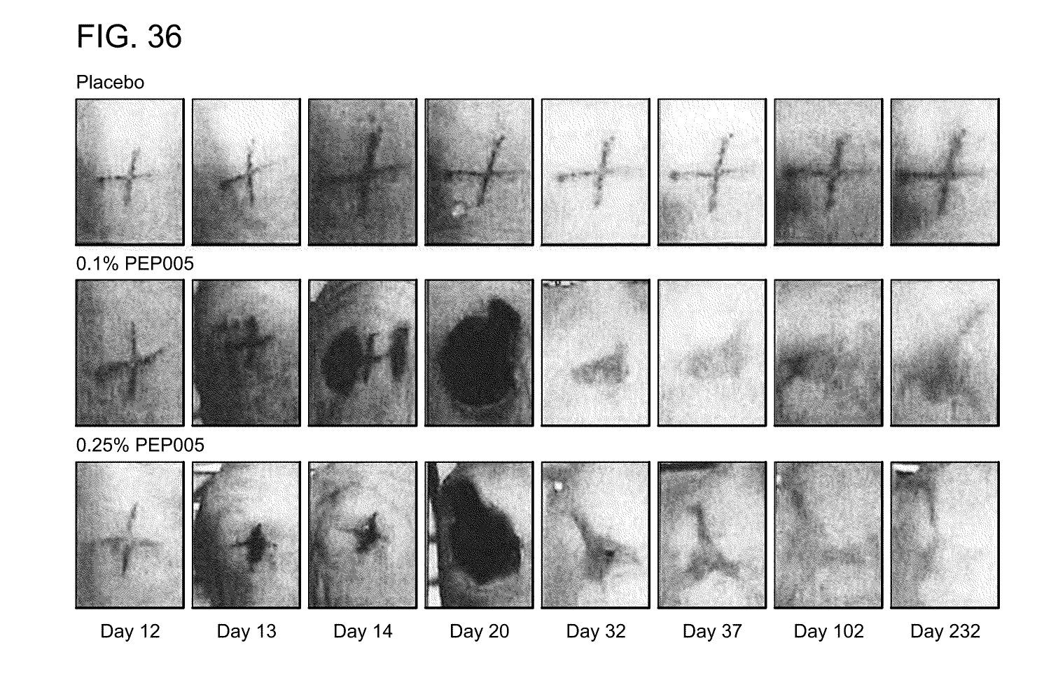

FIG. 36 shows photographs of mice before and after treatment. Mice (n=5 per group) ae tattooed on Day 0 with a cross (˜1 cm×˜1 cm) on their dorsa. Twelve days after the cross is tattooed the mice are topically treated daily for 2 days with about 50 μl of placebo gel, 0.1% PEP005 gel or 0.25% PEP005 gel (Days 12 & 13). The gel treatments are applied onto the tattoo and spread to ensure that the gel covers the entire tattoo marking. The mice are examined weekly to assess any changes to the tattooed area and photographs taken.

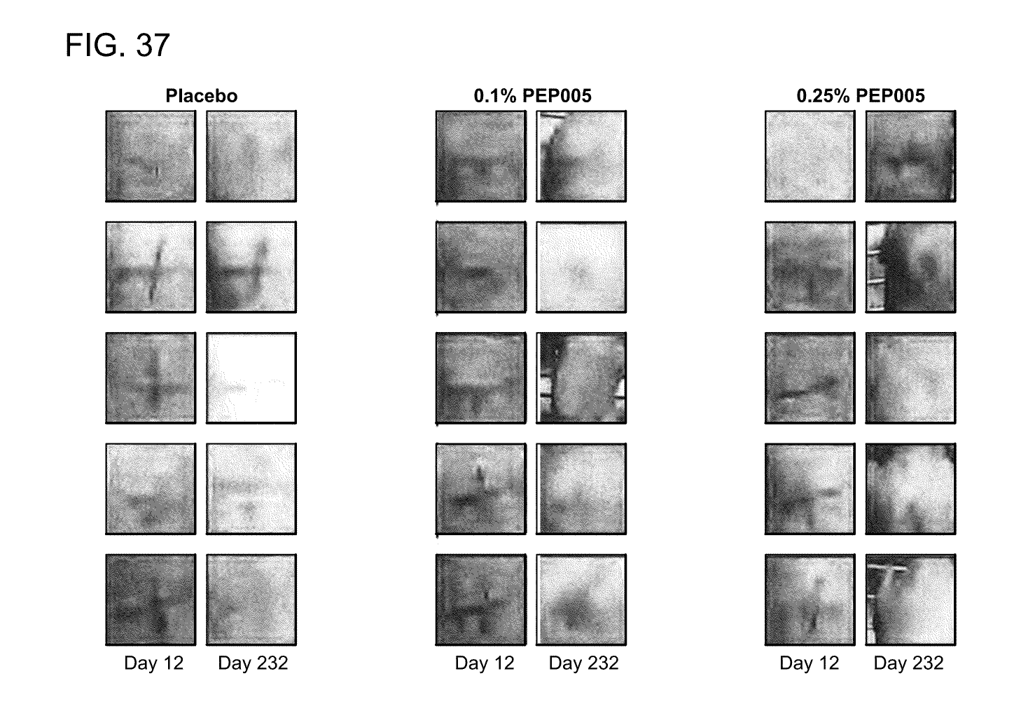

FIG. 37 Shows Mice Treated with Placebo, 0.1% or 0.25% PEP005.

Mice (n=5 per group) are tattooed on Day 0 with a cross (˜1 cm×˜1 cm) on their dorsa. Twelve days after the cross is tattooed, the mice are topically treated daily for 2 days with 50 μl of placebo gel, 0.1% PEP005 gel or 0.25% PEP005 gel (Days 12 & 13). The gel treatments are applied onto the tattoo and spread to ensure that the gel covers the entire tattoo making. The mice are examined weekly to assess any changes to the tattooed area and photographs taken. The photographs are taken prior to first treatment (Day 12) and at termination of experiment (Day 232).

### 3. Conclusions & Recommendations

Treatment of a tattooed area with 0.1% & 0.25% PEP005 daily for 2 days results in the removal of the tattoo markings.

### Example 7

Examples of ingenol mebutate gel formulations contemplated by the present invention and that are used in the Examples 1-6 above are as follows:

| Materials | Target Concentration % w/w |
|---|---|
| 0.01% PEP005 | |
| PEP005 | 0.01 |
| Benzyl Alcohol | 0.9 |
| Citrate Buffer pH 2.75 | 67.59 |
| Isopropyl Alcohol | 30 |
| Hydroxyethyl Cellulose (HX grade) | 1.5 |
| 0.05% PEP005 | |
| PEP005 | 0.05 |
| Benzyl Alcohol | 0.9 |
| Citrate Buffer pH 2.75 | 67.55 |
| Isopropyl Alcohol | 30 |
| Hydroxyethyl Cellulose (HX grade) | 1.5 |
| 0.1% PEP005 | |
| PEP005 | 0.1 |
| Benzyl Alcohol | 0.9 |
| Citrate Buffer pH 2.75 | 67.5 |
| Isopropyl Alcohol | 30 |
| Hydroxyethyl Cellulose (HX grade) | 1.5 |
| 0.25% PEP005 | |
| PEP005 | 0.25 |
| Benzyl Alcohol | 0.9 |
| Citrate Buffer pH 2.75 | 67.35 |
| Isopropyl Alcohol | 30 |
| Hydroxyethyl Cellulose (HX grade) | 1.5 |
| Placebo Gel | |
| PEP005 | 0 |
| Benzyl Alcohol | 0.9 |
| Citrate Buffer pH 2.75 | 67.6 |
| Isopropyl Alcohol | 30 |
| Hydroxyethyl Cellulose (HX grade) | 1.5 |

### REFERENCES

(1) Abdel-Daim M, Funasaka Y, Kamo T et al. (2010) Preventive effect of chemical peeling on ultraviolet induced skin tumor formation. J Dermatol Sci 60:21-8.
(2) Ananthaswamy H N, Loughlin S M, Ullrich S E et al. (1998) Inhibition of UV-induced p53 mutations by sunscreens: implications for skin cancer prevention. J Investig Dermatol Symp Proc 3:52-6.
(3) Ananthaswamy H N, Ullrich S E, Kripke M L (2002) Inhibition of UV-induced p53 mutations and skin cancers by sunscreens: implication for skin cancer prevention. Exp Dermatol 11 Suppl 1:40-3.
(4) Anderson L, Schmieder G J, Werschler W P et al. (2009) Randomized, double-blind, double-dummy, vehicle-controlled study of ingenol mebutate gel 0.025% and 0.05% for actinic keratosis. J Am Acad Dermatol 60:934-43.
(5) Argyris T S (1985) Regeneration and the mechanism of epidermal tumor promotion. Crit. Rev Toxicol 14:211-58.
(6) Bailey H H, Kim K, Verma A K et al. (2010) A randomized, double-blind, placebo-controlled phase 3 skin cancer prevention study of {alpha}-difluoromethylornithine in subjects with previous history of skin cancer. Cancer Prey Res (Phila) 3:35-47.
(7) Benjamin C L, Melnikova V O, Ananthaswamy H N (2008) P53 protein and pathogenesis of melanoma and nonmelanoma skin cancer. Adv Exp Med Biol 624:265-82.
(8) Berg R J, van Kranen H J, Rebel H G et al. (1996) Early p53 alterations in mouse skin carcinogenesis by UVB radiation: immunohistochemical detection of mutant p53 protein in clusters of preneoplastic epidermal cells. Proc Natl Acad Sci USA 93:274-8.
(9) Bosset S, Bonnet-Duquennoy M, Bane P et al. (2003) Photoageing shows histological features of chronic skin inflammation without clinical and molecular abnormalities. Br J Dermatol 149:826-35.
(10) Byrne S N, Limon-Flores A Y, Ullrich S E (2008) Mast cell migration from the skin to the draining lymph nodes upon ultraviolet irradiation represents a key step in the induction of immune suppression. J Immunol 180:4648-55.
(11) Challacombe J M, Suhrbier A, Parsons P G et al. (2006) Neutrophils are a key component of the antitumor efficacy of topical chemotherapy with ingenol-3-angelate. Immunol 177:8123-32.
(12) Chang N B, Feng R, Gao Z et al. (2010) Skin cancer incidence is highly associated with ultraviolet-B radiation history. Int J Hyg Environ Health 213:359-68.
(13) Chaquour B, Seite S, Coutant K et al. (1995) Chronic UVB- and all-trans retinoic-acid-induced qualitative and quantitative changes in hairless mouse skin. J Photochem Photobiol B 28:125-35.
(14) Cohen J L (2010) Actinic keratosis treatment as a key component of preventive strategies for nonmelanoma skin cancer. J Clin Aesthet Dermatol 3:39-44.
(15) Conney A H, Kramata P, Lou Y R et al. (2008) Effect of caffeine on UVB-induced carcinogenesis, apoptosis, and the elimination of UVB-induced patches of p53 mutant epidermal cells in SKH-1 mice. Photochem Photobiol 84:330-8.
(16) de Graaf Y G, Rebel H, Elghalbzouri A et al. (2008) More epidermal p53 patches adjacent to skin carcinomas in renal transplant recipients than in immunocompetent patients: the role of azathioprine. Exp Dermatol 17:349-55.
(17) de Gruijl F R (2008) UV-induced immunosuppression in the balance. Photochem Photobiol 84:2-9.
(18) de Gruijl F R, Rebel H (2008) Early events in UV carcinogenesis—DNA damage, target cells and mutant p53 foci. Photochem Photobiol 84:382-7.
(19) Einspahr J G, Alberts D S, Warneke J A et al. (1999) Relationship of p53 mutations to epidermal cell proliferation and apoptosis in human UV-induced skin carcinogenesis. Neoplasia 1:468-75.
(20) Gonzalez S, Moran M, Kochevar I E (1999) Chronic photodamage in skin of mast cell-deficient mice. Photochem Photobiol 70:248-53.
(21) Hardin J, Mydlarski P R (2010) Systemic retinoids: chemoprevention of skin cancer in transplant recipients. Skin Therapy Lett 15:1-4.
(22) Hart P H, Grimbaldeston M A, Finlay-Jones J J (2001) Sunlight, immunosuppression and skin cancer: role of histamine and mast cells. Clin Exp Pharmacol Physiol 28:1-8.
(23) Haschek W M, Rousseaux C G, Wallig M A (eds) (2009) “Epidermal Lesions” in “Fundamentals of Toxicologic Pathology” Academic Press.
(24) Jonason A S, Kunala S, Price G J et al. (1996) Frequent clones of p53-mutated keratinocytes in normal human skin. Proc Natl Acad Sci USA 93:14025-9.
(25) Kligman L H, Kligman A M (1981) Histogenesis and progression in ultraviolet light-induced tumors in hairless mice. J Natl Cancer Inst 67:1289-93.
(26) Kligman L H, Murphy G F (1996) Ultraviolet B radiation increases hairless mouse mast cells in a dose-dependent manner and alters distribution of UV-induced mast cell growth factor. Photochem Photobiol 63:123-7.
(27) Lau K, Paus R, Tiede S et al. (2009) Exploring the role of stem cells in cutaneous wound healing. Exp Dermatol 18:921-33.
(28) Le T T, Gardner J, Hoang-Le D et al. (2009) Immunostimulatory cancer chemotherapy using local ingenol-3-angelate and synergy with immunotherapies. Vaccine 27:3053-62.
(29) Li L, Shukla S, Lee A et al. (2010) The skin cancer chemotherapeutic agent ingenol-3-angelate (PEP005) is a substrate for the epidermal multidrug transporter (ABCB1) and targets tumor vasculature. Cancer Research 70:4509-19.
(30) Madan V, Lear J T, Szeimies R M (2010) Non-melanoma skin cancer. Lancet 375:673-85. Marcil I, Stern R S (2000) Risk of developing a subsequent nonmelanoma skin cancer in patients with a history of nonmelanoma skin cancer: a critical review of the literature and meta-analysis. Arch Dermatol 136:1524-30.
(31) Marquez C, Bair S M, Smithberger E et al. (2010) Systemic retinoids for chemoprevention of non-melanoma skin cancer in high-risk patients. J Drugs Dermatol 9:753-8. Murphy G M (2009) Ultraviolet radiation and immunosuppression. Br J Dermatol 161 Suppl 3:90-5.
(32) Norval M, Cullen A P, de Gruijl F R et al. (2007) The effects on human health from stratospheric ozone depletion and its interactions with climate change. Photochem Photobiol Sci 6:232-51.
(33) Ogbourne S M, Hampson P, Lord J M et al. (2007) Proceedings of the First International Conference on PEP005. Anticancer Drugs 18:357-62.
(34) Rass K, Reichrath J (2008) UV damage and DNA repair in malignant melanoma and nonmelanoma skin cancer. Adv Exp Med Biol 624:162-78.
(35) Rebel H, Kram N, Westerman A et al. (2005) Relationship between UV-induced mutant p53 patches and skin tumours, analysed by mutation spectra and by induction kinetics in various DNA-repair-deficient mice. Carcinogenesis 26:2123-30.
(36) Rebel H, Mosnier L O, Berg R J et al. (2001) Early p53-positive foci as indicators of tumor risk in ultraviolet-exposed hairless mice: kinetics of induction, effects of DNA repair deficiency, and p53 heterozygosity. Cancer Res 61:977-83.
(37) Remenyik E, Wikonkal N M, Zhang W et al. (2003) Antigen-specific immunity does not mediate acute regression of UVB-induced p53-mutant clones. Oncogene 22:6369-76. Rogers H W, Weinstock M A, Harris A R et al. (2010) Incidence estimate of nonmelanoma skin cancer in the United States, 2006. Arch Dermatol 146:283-7.
(38) Schmieder G (2010) Multicenter, open-label, dose-area escalation, cohort study to evaluate the safety and tolerability of PEP005 (ingenol mebutate) Gel, 0.05% applied for 2 consecutive days to treatment area(s) of up to a total of 100 cm2 in patients with actinic keratoses (AK) on the extensor (dorsal aspect) forearm(s). American Academy of Dermatology Annual Meeting:Poster presentation.
(39) Siller G, Gebauer K, Welburn P et al. (2009) PEP005 (ingenol mebutate) gel, a novel agent for the treatment of actinic keratosis: results of a randomized, double-blind, vehicle-controlled, multicentre, phase IIa study. Australas J Dermatol 50:16-22. Siller G, Rosen R, Freeman M et al. (2010) PEP005 (ingenol mebutate) gel for the topical treatment of superficial basal cell carcinoma: results of a randomized phase IIa trial. Australas J Dermatol 51:99-105.
(40) Smith K, Hamza S, Germain M et al. (2007) Does imiquimod histologically rejuvenate ultraviolet radiation-damaged skin? Dermatol Surg 33:1419-28; discussion 28-9. American Cancer Society (2010) Cancer Facts & FIGS. 2010. American Cancer Society, Atlanta, USA.
(41) The Lewin Group, Inc. (2005) The Burden of Skin Diseases. Prepared for: The Society for Investigative Dermatology and The American Academy of Dermatology Association. Ziegler A, Jonason A S, Leffell D J et al. (1994) Sunburn and p53 in the onset of skin cancer. Nature 372:773-6.
(42) Ambler, C. A. and A. Maatta: Epidermal stem cells: location, potential and contribution to cancer. J. Pathol. Dev. 217, 206-216 (2009)
(43) Benjamin, C. L., S. E. Ullrich, M. L. Kripke and H. N. Ananthaswamy: p53 tumor suppressor gene: a critical molecular target for UV induction and prevention of skin cancer. Photochem. Photobiol. Dev. 84, 55-62 (2008)
(44) Brand, C., M. A. Grimbaldeston, J. R. Gamble, J. Drew, J. J. Finlay-Jones and P. H. Hart: Tea tree oil reduces the swelling associated with the efferent phase of a contact hypersensitivity response. Inflamm. Res. Dev. 51, 236-244 (2002)
(45) Brash, D. E., J. A. Rudolph, J. A. Simon, A. Lin, G. J. McKenna, H. P. Baden, A. J. Halperin and J. Ponten: A role for sunlight in skin cancer: UV-induced p53 mutations in squamous cell carcinoma. Proc. Natl. Acad. Sci. U.S.A. Dev. 88, 10124-10128 (1991)
(47) Ch'ng, S., R. A. Wallis, L. Yuan, P. F. Davis and S. T. Tan: Mast cells and cutaneous malignancies. Mod. Pathol. Dev. 19, 149-159 (2006)
(48) Chaquour, B., S. Seite, K. Coutant, A. Fourtanier, J. P. Borel and G. Belton: Chronic UVB- and alltrans retinoic-acid-induced qualitative and quantitative changes in hairless mouse skin. J. Photochem. Photobiol. B Dev. 28, 125-135 (1995)
(49) Cozzi, S-J and Suhrbier, A. Establishing the SKH1 model of UV-induced p53+ mutant patches at QIMR. QIMR 07-085. 2010. Queensland Institute of Medical Research, Brisbane, QLD 4006, Australia. (GENERIC) Ref Type: Report de Gruijl, F. R. and H. Rebel: Early events in UV carcinogenesis—DNA damage, target cells and mutant p53 foci. Photochem. Photobiol. Dev. 84, 382-387 (2008)
(50) Einspahr, J. G., D. S. Alberts, J. A. Warneke, P. Bozzo, J. Basye, T. M. Grogan, M. A. Nelson and G. T. Bowden: Relationship of p53 mutations to epidermal cell proliferation and apoptosis in human UV-induced skin carcinogenesis. Neoplasia. Dev. 1, 468-475 (1999)
(51) Einspahr, J. G., M. J. Xu, J. Warneke, K. Saboda, J. Ranger-Moore, P. Bozzo, L. Duckett, R. Goldman, P. Lin, J. Buckmeier and D. S. Alberts: Reproducibility and expression of skin biomarkers in sun-damaged skin and actinic keratoses. Cancer Epidemiol. Biomarkers Prey. Dev. 15, 1841-1848 (2006)
(52) Gabrilovich, D. I. and S, Nagaraj: Myeloid-derived suppressor cells as regulators of the immune system. Nat. Rev. Immunol. Dev. 9, 162-174 (2009)
(53) Gerdes, M. J. and S. H. Yuspa: The contribution of epidermal stem cells to skin cancer. Stem Cell Rev. Dev. 1, 225-231 (2005)
(54) Hart, P. H., M. A. Grimbaldeston and J. J. Finlay-Jones: Mast cells in UV-B-induced immunosuppression. J. Photochem. Photobiol. B Dev. 55, 81-87 (2000)
(55) Hart, P. H., M. A. Grimbaldeston and J. J. Finlay-Jones: Sunlight, immunosuppression and skin cancer: role of histamine and mast cells. Clin. Exp. Pharmacol. Physiol Dev. 28, 1-8 (2001)
(56) Morris, R. J., K. A. Tryson and K. Q. Wu: Evidence that the epidermal targets of carcinogen action are found in the interfollicular epidermis of infundibulum as well as in the hair follicles. Cancer Res. Dev. 60, 226-229 (2000)
(57) Natarajan, E., J. D. Omobono, Z. Guo, S. Hopkinson, A. J. Lazar, T. Brenn, J. C. Jones and J. G. Rheinwald: A keratinocyte hypermotility/growth-arrest response involving laminin 5 and p161NK4A activated in wound healing and senescence. Am. J. Pathol. Dev. 168, 1821-1837 (2006)
(58) Nelson, M. A., J. G. Einspahr, D. S. Alberts, C. A. Balfour, J. A. Wymer, K. L. Welch, S. J. Salasche, J. L. Bangert, T. M. Grogan and P. O. Bozzo: Analysis of the p53 gene in human precancerous actinic keratosis lesions and squamous cell cancers. Cancer Lett. Dev. 85, 23-29 (1994)
(59) Ogbourne, S. M., A. Suhrbier, B. Jones, S. J. Cozzi, G. M. Boyle, M. Morris, D. McAlpine, J. Johns, T. M. Scott, K. P. Sutherland, J. M. Gardner, T. T. Le, A. Lenarczyk, J. H. Aylward and P. G. Parsons: Antitumor activity of 3-ingenyl angelate: plasma membrane and mitochondrial disruption and necrotic cell death. Cancer Res. Dev. 64, 2833-2839 (2004)
(60) Rebel, H., L. O. Mosnier, R. J. Berg, V. A. Westerman-de, S. H. van, H. J. van Kranen and F. R. de Gruijl: Early p53-positive foci as indicators of tumor risk in ultraviolet-exposed hairless mice: kinetics of induction, effects of DNA repair deficiency, and p53 heterozygosity. Cancer Res. Dev. 61, 977-983 (2001)
(61) Moreno G, Chia A L, Lim A, Shumack S. Australas J Dermatol 2007; 48: 1-8.
(62) Cox N H, Eedy D J, Morton C A; Therapy Guidelines and Audit Subcommittee, British Association of Dermatologists. Br J Dermatol 2007; 156: 11-21.
(63) Kossard S, Rosen R H. J Am Acad Dermatol 1992; 27: 406-10.
(64) Ball S B, Dawber R P. Australas J Dermatol 1998; 39: 63-8.
(65) Green A C, Beardmore G L. Australas J Dermatol 1988; 29: 127-30.
(66) Ogboume S M, Suhrbier A, Jones B et al. Cancer Res 2004; 64: 2833-9.
(67) Challacombe J M, Suhrbier A, Parsons P G et al. J Immunol 2006; 177: 8123-32.
(68) Siller G et al. Ingenol-3-angelate (PEP005) gel, a novel treatment for actinic keratoses: results of a randomised double-blind placebo-controlled multicentre safety and efficacy study.
(69) Anderson L, Welbum P. Maximum tolerated dose of PEP005 Topical Gel for the treatment of actinic keratosis. American Academy of Dermatology Summer Academy Meeting 2007. Poster number 1503.
(70) Rosen R H, Freeman M, Siller G, Katsamas J, Ogbourne SM. Safety and efficacy of PEP005 Topical Gel for the treatment of nodular and superficial forms of basal cell carcinoma. American Academy of Dermatology Summer Academy Meeting 2007. Poster number 1502.
(71) The Harris Poll #15: Three in Ten Americans with a Tattoo Say Having One Makes Them Feel Sexier, Feb. 12, 2008.
(72) Harris Interactive Europe poll: Americans and Britons Likelier Than Italians To Regret Decision to be Tattooed, Feb. 5, 2004.
(73) S. Varma, et al.: Tattoo ink darkening of a yellow tattoo after Q-switched laser treatment. Clinical and Experimental Dermatology. 2002: Volume 27 Issue 6, pp. 461-463; and Holzer A, et al. Adverse Effects of Q-Switched Laser Treatment of Tattoos. Dermatologic Surgery 2007: Volume 34 Issue 1, pp. 118-122.
(74) Taylor Charles R.: Laser ignition of traumatically embedded firework debris, Lasers in Surgery and Medicine, 1998/22:157-158
(75) Challacombe J M, Suhrbier A, Parsons P G, Jones B, Hampson P, Kavanagh D, Rainger G E, Morris M, Lord J M, Le T T, Hoang-Le D, Ogboume S M (2006) Neutrophils are a key component of the antitumor efficacy of topical chemotherapy with ingenol-3-angelate. J Immunol 177:81238132
(76) Le T T, Gardner J, Hoang-Le D, Schmidt C W, MacDonald K P, Lambley E, Schroder W A, Ogbourne S M, Suhrbier A (2009) Immunostimulatory cancer chemotherapy using local ingenol-3-angelate and synergy with immunotherapies. Vaccine 27:3053-3062
(77) Li L, Shukla S, Lee A, Garfield S H, Maloney D J, Ambudkar S V, Yuspa S H (2010) The skin cancer chemotherapeutic agent ingenol-3-angelate (PEP005) is a substrate for the epidermal multidrug transporter (ABCB1) and targets tumor vasculature. Cancer Res 70:4509-4519
(78) Ogboume S M, Suhrbier A, Jones B, Cozzi S J, Boyle G M, Morris M, McAlpine D, Johns J, Scott T M, Sutherland K P, Gardner J M, Le T T, Lenarczyk A, Aylward J H, Parsons P G (2004) Antitumor activity of 3-ingenyl angelate: plasma membrane and mitochondrial disruption and necrotic cell death. Cancer Res 64:2833-2839
(79) How Young People View Their Lives, Futures and Politics. A Portrait of “Generation Next”. The Pew Research Centre for the People and the Press 2007 Jan. 9 Available from: URL: http://people-press.org/report/300/a-portrait-of-generation-next
(80) Regina a.Corso. Three in Ten Americans with a Tattoo Say Having One Makes Them Feel Sexier. Harris Interactive 2010 Feb. 12 Available from: URL: http://www.harrisinteractive.com/vault/Harris-Interactive-Poll-Research-Three-in-Ten-Americans-with-a-Tattoo-Sav-Havino-One-Makes-Them-Feel-Sexier-2008-02.pdf
(81) Suzanne L. Kilmer, Richard E. Fitzpatrick, Michael P. Goldman. Tattoo lasers. emedicine 2008 Jun. 25. Available from: URL: www.emedicine.com/derm/topic563.htm
(82) Pfirrmann G, Karsai S, Roos S, Hammes S, Raulin C. Tattoo removal—state of the art. J Dtsch Dermatol Ges 2007 October; 5(10):889-97
(83) Burris K, Kim K. Tattoo removal. Clin Dermatol 2007 July; 25(4):388-92
(84) Pfirrmann G, Karsai S, Roos S, Hammes S, Raulin C. Tattoo removal—state of the art. J Dtsch Dermatol Ges 2007 October; 5(10):889-97
(85) Pfirrmann G, Karsai S, Roos S, Hammes S, Raulin C. Tattoo removal—state of the art. J Dtsch Dermatol Ges 2007 October; 5(10):889-97
(86) Ramirez M, Magee N, Diven D, Colome-Grimmer M, Motamedi M, Oliveira G, et al. Topical imiquimod as an adjuvant to laser removal of mature tattoos in an animal model. Dermatol Surg 2007 March; 33(3):319-25
(87) Elsaie M L, Nouri K, Vejjabhinanta V, Rivas M P, Villafradez-Diaz L M, Martins A, et al. Topical imiquimod in conjunction with Nd:YAG laser for tattoo removal. Lasers Med Sci 2009 November; 24(6):871-5
(88) Sarah-Jane Cozzi, Andreas Suhrbier. Removal of p53 mutant cells by PEP005 treatment

The attached Appendix B forms a part of this disclosure and is incorporated herein by reference in its entirety.

Having described our invention, we claim:

1. A method of treating UV-damaged skin in a subject in need thereof by applying an effective amount of ingenol mebutate to the UV-damaged skin to reduce the number of skin lesions that will emerge from the UV-damaged skin, or prevent development of skin lesions or skin cancers in the UV-damaged skin.

2. The method of claim 1, wherein ingenol mebutate is applied in a concentration of 0.05%.

3. The method of claim 1, wherein ingenol mebutate is applied once daily or two consecutive days.

4. The method of claim 1, wherein ingenol mebutate is administered in the form of a gel.

5. The method of claim 1, wherein the patient has undergone prior treatment for actinic keratosis or skin cancer.

6. The method of claim 1, wherein the patient is treated as a field-directed treatment in areas surrounding the actinic keratosis or skin cancer.

7. The method of claim 1, wherein the patient has not been diagnosed with any skin cancer or actinic keratosis.

8. (canceled)

9. A method of treating SCC tumors in a subject in need thereof by applying an effective amount of ingenol mebutate to the SCC tumors to reduce the number of SCC tumors.

10. The method of claim 1, wherein ingenol mebutate is applied in a concentration of 0.25%.

11.-22. (canceled)

23. A topical method for removing a tattoo on skin of a subject with a gel formulated with ingenol mebutate, the method comprises:
applying the gel once daily for up to two consecutive days to the tattoo to topically deliver an effective amount of ingenol mebutate to remove the tattoo.

24. A topical method of claim 23, wherein the gel contains ingenol mebutate in an amount of up to about 0.25% by weight.

25. A method of treating UV-damaged skin in a subject in need thereof by applying an effective amount of ingenol mebutate to the UV-damaged skin to remove from the UV-damaged skin mutated keratinocytes, cutaneous immunosuppressive environments or p53+ patches.

26. The method of claim 25, wherein ingenol mebutate is applied in a concentration of 0.05%.

27. The method of claim 25, wherein the ingenol mebutate is applied once daily or two consecutive days.

28. The method of claim 25, wherein ingenol mebutate is administered in the form of a gel.

29. The method of claim 25, wherein the patient has undergone prior treatment for actinic keratosis or skin cancer.

30. (canceled)

31. A topical method of treating photodamaged skin comprising:
applying topically an effective amount of ingenol mebutate to photodamaged skin to regenerate an epithelial thickness that is similar to unirradiated skin that has not been photodamaged, or is significantly less than that seen in UV-damaged skin.

32. (canceled)

33. A topical method of treating UV-damaged skin comprising:
applying topically an effective amount of ingenol mebutate to UV-damaged skin to prevent the formation of cancer cells from the UV-damaged skin or to remove the UV-damaged skin and generate a new epithelial layer of skin.

34. (canceled)

35. A topical method of claim 33, wherein the gel contains ingenol mebutate in an amount of about 0.25% by weight.

36. A topical method of claim 33, wherein the gel contains ingenol mebutate in an amount of about 0.10% by weight.

37. A topical method of claim 33, wherein the gel contains ingenol mebutate in an amount of about 0.05% by weight.
